(19) United States
(12) Patent Application Publication
(10) Pub. No.: US 2017/0247436 A1
(43) Pub. Date: Aug. 31, 2017

(54) NON-HUMAN ANIMALS HAVING A MUTANT KYNURENINASE GENE

(71) Applicant: Regeneron Pharmaceuticals, Inc., Tarrytown, NY (US)

(72) Inventors: Christos Kyratsous, Irvington, NY (US); Alexander O. Mujica, Elmsford, NY (US)

(21) Appl. No.: 15/435,134

(22) Filed: Feb. 16, 2017

Related U.S. Application Data
(60) Provisional application No. 62/295,524, filed on Feb. 16, 2016.

(51) Int. Cl. C07K 16/10; A01K 67/027; C12N 9/14

(52) U.S. Cl. CPC C07K 16/1081; C12Y 307/01003; C12N 9/14; A01K 67/0278; A01K 2267/01; A01K 2227/105; A01K 2217/075; C07K 2317/56; C07K 2317/52; C07K 2317/10; C07K 2317/34; C07K 2317/21

(57) ABSTRACT

Non-human animals, methods and compositions for making and using the same, are provided, wherein said non-human animals comprise a mutant L-kynurenine hydrolase (or kynureninase) gene. Said non-human animals may be described, in some embodiments, as having a genetic modification in an endogenous kynureninase gene so that said non-human animals express a kynureninase polypeptide that includes an amino acid substitution that results in the elimination of an epitope in said kynureninase polypeptide that is present in the membrane proximal external region of human immunodeficiency virus-1 gp41.

### CROSS-REFERENCE TO RELATED APPLICATIONS

This application claims priority to U.S. Provisional Patent Application No. 62/295,524, filed Feb. 16, 2016, the disclosure of which is incorporated by reference herein in its entirety.

### FIELD OF INVENTION

Non-human animals comprising a mutant L-kynurenine hydrolase (or kynureninase) gene. Non-human animals that express mutant L-kynurenine hydrolase proteins. Methods for making and using non-human animals comprising mutant L-kynurenine hydrolase nucleic acid sequences.

### BACKGROUND

According to the World Health Organization (WHO), human immunodeficiency virus (HIV) is a major global health issue that has claimed over 34 million lives. In particular, global HIV-related deaths were estimated at 980,000 to 1.6 million in 2014. HIV infects critical cells of the immune system; in particular, CD4⁺ T cells, and over time weakens a host's defense against various infections and cancer leading to a condition known as acquired immune deficiency syndrome (AIDS). Despite the development of various anti-viral treatments that have shown promise in controlling HIV infection and prevention of further transmission, there is no cure. Recently, HIV has been implicated to evade host immune surveillance by immunological tolerance thereby impairing immune responses (e.g., antibody responses) to neutralizing HIV epitopes that are similar to self-antigens.

### SUMMARY

The present invention encompasses the recognition that it is desirable to engineer non-human animals to permit improved in vivo systems for identifying and developing new therapeutics and, in some embodiments, therapeutic regimens, which can be used for the treatment and/or prevention of HIV infection and/or transmission. In some embodiments, in vivo systems described herein can be used for identifying and developing new therapeutics for treating hypertension and/or renal disease. Provided non-human animals comprise a disruption in a Kynureninase (Kynu) gene and/or otherwise functionally silenced Kynu gene, such that a host Kynu polypeptide is not expressed or produced, and are desirable, for example, for use in identifying and developing therapeutics that target HIV (e.g., HIV infection, transmission, replication, and/or HIV serum levels). Non-human animals are also provided that comprise a mutant Kynu gene such that a variant Kynu polypeptide is expressed or produced by said mutant Kynu gene, and are desirable, for example, for use in identifying and developing therapeutics that target HIV (e.g., HIV infection, transmission, replication, and/or HIV serum levels). In some embodiments, non-human animals as described herein provide improved in vivo systems (or models) for HIV-related diseases, disorders and conditions. In some embodiments, non-human animals described herein provide improved in vivo systems (or models) for hypertensive disease, disorders, and conditions.

The present invention provides methods for producing antibodies that bind an epitope that is shared between a foreign antigen (e.g., a pathogen) and a self-antigen. In particular, the present invention provides a method for producing an antibody or fragment thereof that binds a shared epitope of a foreign antigen and a self-antigen, the method comprising the steps of immunizing a non-human animal with an antigen that contains an epitope shared with or present on (or substantially identical or identical to) a foreign antigen and a self-antigen, maintaining the non-human animal under conditions sufficient that the non-human animal produces an immune response to the epitope shared with or present on the foreign antigen and the self-antigen, and recovering an antibody from the non-human animal, or a non-human animal cell, that binds the epitope shared with or present on the foreign antigen and the self-antigen, wherein the non-human animal has a genome comprising a disruption or mutation in a gene that results in the elimination of an epitope from a self-antigen that is shared with, present on or appears in a foreign antigen that is not a homolog of the self-antigen. In various embodiments, a foreign antigen is a virus (e.g., HIV). In various embodiments, methods for producing antibodies described herein further comprise obtaining genetic material from an immunized non-human animal (or non-human cell), and producing an antibody or fragment thereof that binds a shared epitope from the genetic material.

In some embodiments, a disruption is or comprises a homozygous deletion, in whole or in part, of a gene that eliminates expression of the gene product (e.g., mRNA or polypeptide). In some embodiments, a mutation is or comprises one or more point mutations in a gene that eliminates expression of an epitope in the gene product that is shared with or present in (or substantially identical or identical to) a foreign antigen such as, for example, a pathogen (e.g., a virus, bacterium, prion, fungus, viroid, or parasite).

In some embodiments, the present invention provides non-human animals having a genome comprising an engineered Kynu gene, which engineered Kynu gene includes one or more mutations as compared to a wild-type Kynu gene (e.g., endogenous or homolog) that results in the expression of a variant Kynu polypeptide. In some embodiments, such an engineered Kynu gene includes genetic material that encodes an H4 domain of a rodent Kynu polypeptide, which H4 domain contains an amino acid substitution as compared to a wild-type or parental rodent Kynu polypeptide. Thus, in some embodiments, an engineered Kynu gene of a non-human animal as described herein encodes a Kynu polypeptide characterized by an H4 domain that includes an amino acid substitution (e.g., a variant Kynu polypeptide).

In some embodiments, the present invention provides non-human animals having a genome comprising an engineered Kynu gene as described herein and an engineered immunoglobulin heavy and/or light chain locus, which engineered immunoglobulin heavy and/or light chain locus comprises genetic material from two different species (e.g., a human portion and a non-human portion). In some embodiments, such an engineered immunoglobulin heavy and/or light chain locus includes genetic material that encodes one or more immunoglobulin variable regions (i.e., assembled V, D and/or J segments). In some embodiments, genetic material encodes immunoglobulin heavy and/or light chain variable domains that are responsible for antigen-binding. Thus, in some embodiments, an engineered immunoglobulin heavy and/or light chain locus of a non-human animal as described herein encodes immunoglobulin heavy and/or light chain domains that contain human and non-human portions, wherein the human and non-human portions are linked together and form a functional immunoglobulin heavy and/or light chain of an antibody.

In some embodiments, a non-human animal is provided whose genome comprises a mutant kynureninase (Kynu) gene, which mutant Kynu gene comprises one or more point mutations in exon three that results in (or encodes) a Kynu polypeptide having a D93E substitution.

In some embodiments, a non-human animal is provided that expresses a Kynu polypeptide that includes a D93E substitution.

In some embodiments, a mutant Kynu gene comprises 1, 2, 3, 4 or 5 point mutations; in some certain embodiments, 5 point mutations in exon three. In some embodiments, a mutant Kynu gene further comprises a deletion in intron three that results from insertion of (or homologous recombination with) a selection cassette; in some certain embodiments, a deletion is about 60 bp. In some embodiments, a mutant Kynu gene further comprises one or more selection markers. In some embodiments, a mutant Kynu gene further comprises one or more site-specific recombinase recognition sites. In some embodiments, a mutant Kynu gene comprises a recombinase gene and a selection marker flanked by recombinase recognition sites, which recombinase recognition sites are oriented to direct an excision. In some embodiments, a mutant Kynu gene comprises an exon three that includes the sequence that appears in SEQ ID NO:42 or encodes a Kynu polypeptide comprising the sequence that appears in SEQ ID NO:36 or SEQ ID NO:41.

In some embodiments, a recombinase gene is operably linked to a promoter that drives expression of the recombinase gene in differentiated cells and does not drive expression of the recombinase gene in undifferentiated cells. In some embodiments, a recombinase gene is operably linked to a promoter that is transcriptionally competent and developmentally regulated. In some embodiments of a promoter that is transcriptionally competent and developmentally regulated, the promoter is or comprises SEQ ID NO:37, SEQ ID NO:38, or SEQ ID NO:39. In some embodiments of a promoter that is transcriptionally competent and developmentally regulated, the promoter is or comprises SEQ ID NO:37.

In some embodiments, a provided non-human animal is homozygous for a mutant Kynu gene as described herein. In some embodiments, a provided non-human animal is heterozygous for a mutant Kynu gene as described herein. In some embodiments, a provided non-human animal is hemizygous (i.e., has one copy) for a mutant Kynu gene as described herein.

In some embodiments, the genome of a provided non-human animal further comprises an insertion of a human immunoglobulin heavy chain variable region that includes one or more human V_{H} segments, one or more human D_{H} segments and one or more human J_{H} segments, which human immunoglobulin heavy chain variable region is operably linked to an immunoglobulin heavy chain constant region.

In some embodiments, an immunoglobulin heavy chain constant region is a rodent immunoglobulin heavy chain constant region; in some certain embodiments, an endogenous rodent immunoglobulin heavy chain constant region.

In some embodiments, the genome of a provided non-human animal further comprises an insertion of a human immunoglobulin light chain variable region that includes one or more human V_{L} segments and one or more human J_{L} segments, which human immunoglobulin light chain variable region is operably linked to an immunoglobulin light chain constant region.

In some embodiments, an immunoglobulin light chain constant region is a rodent immunoglobulin light chain constant region; in some certain embodiments, an endogenous rodent immunoglobulin light chain constant region. In some embodiments, human V_{L} and J_{L} segments are human Vκ and Jκ segments and are inserted into an endogenous κ light chain locus; in some certain embodiments, human Vκ and Jκ segments are operably linked to an endogenous rodent Cκ gene. In some embodiments, human V_{L} and J_{L} segments are human Vλ and Jλ segments and are inserted into an endogenous λ light chain locus; in some certain embodiments, human Vλ and Jλ segments are operably linked to an endogenous rodent Cλ gene.

In some embodiments, a provided non-human animal expresses a Kynu polypeptide as described herein and further expresses antibodies comprising human variable domains and non-human (e.g., rodent) constant domains. In some embodiments, human variable domains include human V_{H} and Vκ domains. In some certain embodiments, human Vκ domains are fused to rodent Cκ domains.

In some embodiments, an isolated non-human cell or tissue is provided whose genome comprises a mutant Kynu gene (or locus) as described herein. In some embodiments, a cell is a lymphocyte. In some embodiments, a cell is selected from a B cell, dendritic cell, macrophage, monocyte, and a T cell. In some embodiments, a tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof.

In some embodiments, an immortalized cell made, generated, produced or obtained from an isolated non-human cell or tissue as described herein is provided.

In some embodiments, a non-human embryonic stem (ES) cell is provided whose genome comprises a mutant Kynu gene (or locus) as described herein. In some embodiments, a non-human embryonic stem cell is a rodent embryonic stem cell. In some certain embodiments, a rodent embryonic stem cell is a mouse embryonic stem cell and is from a 129 strain, C57BL strain, or a mixture thereof. In some certain embodiments, a rodent embryonic stem cell is a mouse embryonic stem cell and is a mixture of 129 and C57BL strains. In some embodiments, a non-human ES cell as described herein comprises any one of SEQ ID NO:9, SEQ ID NO:10, SEQ ID NO:11, SEQ ID NO:12, SEQ ID NO:13 and SEQ ID NO:14. In some embodiments, a non-human ES cell as described herein comprises SEQ ID NO:15 and SEQ ID NO:16, SEQ ID NO:15 and SEQ ID NO:17, SEQ ID NO:24 and SEQ ID NO:25, or SEQ ID NO:26.

In some embodiments, use of a non-human embryonic stem cell as described herein to make a non-human animal is provided. In some certain embodiments, a non-human ES cell is a mouse ES cell and is used to make a mouse comprising a mutant Kynu gene (or locus) as described herein. In some certain embodiments, a non-human ES cell is a rat ES cell and is used to make a rat comprising a mutant Kynu gene (or locus) as described herein.

In some embodiments, a non-human embryo made, produced, generated, or obtained from a non-human ES cell as described herein is provided. In some certain embodiments, a non-human embryo is a rodent embryo; in some embodiments, a mouse embryo; in some embodiments, a rat embryo.

In some embodiments, use of a non-human embryo described herein to make a non-human animal is provided. In some certain embodiments, a non-human embryo is a mouse embryo and is used to make a mouse comprising a mutant Kynu gene (or locus) as described herein. In some certain embodiments, a non-human embryo is a rat embryo and is used to make a rat comprising a mutant Kynu gene (or locus) as described herein.

In some embodiments, a kit comprising a non-human animal, an isolated non-human cell or tissue, an immortalized cell, a non-human ES cell, or a non-human embryo as described herein is provided.

In some embodiments, a kit as described herein for use in the manufacture and/or development of a drug (e.g., an antibody or antigen-binding fragment thereof) for therapy or diagnosis is provided.

In some embodiments, a kit as described herein for use in the manufacture and/or development of a drug (e.g., an antibody or antigen-binding fragment thereof) for the treatment, prevention or amelioration of a disease, disorder or condition is provided.

In some embodiments, a nucleic acid construct or targeting vector as described herein is provided. In some certain embodiments, a provided nucleic acid construct or targeting vector comprises a Kynu gene (or locus), in whole or in part, as described herein. In some certain embodiments, a provided nucleic acid construct or targeting vector comprises a DNA fragment that includes a Kynu gene (or locus), in whole or in part, as described herein. In some certain embodiments, a provided nucleic acid construct or targeting vector comprises any one of SEQ ID NO:9, SEQ ID NO:10, SEQ ID NO:12 and SEQ ID NO:13. In some certain embodiments, a provided nucleic acid construct or targeting vector comprises SEQ ID NO:15 and SEQ ID NO:16, or SEQ ID NO:24 and SEQ ID NO:25. In some certain embodiments, a provided nucleic acid construct or targeting vector comprises one or more selection markers. In some certain embodiments, a provided nucleic acid construct or targeting vector comprises one or more site-specific recombination sites (e.g., loxP, Frt, or combinations thereof). In some certain embodiments, a provided nucleic acid construct or targeting vector is depicted in FIG. 2A, 4A or 4C.

In some embodiments, use of a nucleic acid construct or targeting vector as described herein to make a non-human ES cell, non-human cell, non-human embryo and/or non-human animal is provided.

In some embodiments, a method of making a non-human animal whose genome comprises a mutant Kynu gene (or that expresses a Kynu polypeptide that includes a D93E substitution from an endogenous Kynu gene) is provided, the method comprising (a) introducing a nucleic acid sequence into a non-human embryonic stem cell so that exon three of a Kynu gene is mutated to encode (or result in) a Kynu polypeptide that includes a D93E substitution, which nucleic acid comprises a polynucleotide that is homologous to a Kynu locus; (b) obtaining a genetically modified non-human ES cell from (a); and (c) creating a non-human animal using the genetically modified non-human ES cell of (b).

In some embodiments of a method of making a non-human animal whose genome comprises a mutant Kynu gene, the method further comprises a step of breeding the non-human animal generated in (c) so that a non-human animal homozygous for the mutant Kynu gene is created. In some embodiments of a method of making a non-human animal whose genome comprises a mutant Kynu gene, the non-human ES cell of (a) has a genome that comprises (i) an insertion of a human immunoglobulin heavy chain variable region that includes one or more human V_{H} segments, one or more human D_{H} segments and one or more human J_{H} segments, which human immunoglobulin heavy chain variable region is operably linked to an immunoglobulin heavy chain constant region, and/or (ii) an insertion of a human immunoglobulin light chain variable region that includes one or more human V_{L} segments and one or more human J_{L} segments, which human immunoglobulin light chain variable region is operably linked to an immunoglobulin light chain constant region. In some embodiments of a method of making a non-human animal whose genome comprises a mutant Kynu gene, a nucleic acid sequence comprises one or more selection markers and/or one or more site-specific recombinase recognition sites. In some embodiments of a method of making a non-human animal whose genome comprises a mutant Kynu gene, a nucleic acid sequence comprises a recombinase gene and a selection marker flanked by recombinase recognition sites, which recombinase recognition sites are oriented to direct an excision.

In some embodiments, a method of making a non-human animal whose genome comprises a mutant Kynu gene that encodes a Kynu polypeptide that includes a D93E substitution is provided, the method comprising modifying the genome of a non-human animal so that it comprises a mutant Kynu gene that encodes a Kynu polypeptide having a D93E substitution, thereby making said non-human animal.

In some embodiments of a method of making a non-human animal whose genome comprises a mutant Kynu gene, the genome of a non-human animal is modified so that it comprises a mutant Kynu exon three that includes the sequence that appears in SEQ ID NO:42. In some certain embodiments of a method of making a non-human animal whose genome comprises a mutant Kynu gene, the genome of a non-human animal is modified so that it further comprises a deletion in intron three (e.g., about 60 bp).

In some embodiments of a method of making a non-human animal whose genome comprises a mutant Kynu gene, the method further comprises modifying the genome of the non-human animal so that it comprises (i) an insertion of a human immunoglobulin heavy chain variable region that includes one or more human V_{H} segments, one or more human D_{H} segments and one or more human J_{H} segments, which human immunoglobulin heavy chain variable region is operably linked to an immunoglobulin heavy chain constant region, and/or (ii) an insertion of a human immunoglobulin light chain variable region that includes one or more human V_{L} segments and one or more human J_{L} segments, which human immunoglobulin light chain variable region is operably linked to an immunoglobulin light chain constant region. In some certain embodiments, modifying the genome of the non-human animal so that it comprises (i) and/or (ii) is performed prior to modifying the genome of the rodent so that it comprises a mutant Kynu gene that encodes a Kynu polypeptide having a D93E substitution.

In some embodiments of a method of making a non-human animal whose genome comprises a mutant Kynu gene, the method further comprises breeding a non-human animal whose genome comprises a mutant Kynu gene that encodes a Kynu polypeptide having a D93E substitution with a second non-human animal, which second non-human animal has a genome comprising (i) an insertion of a human immunoglobulin heavy chain variable region that includes one or more human V_{H} segments, one or more human D_{H} segments and one or more human J_{H} segments, which human immunoglobulin heavy chain variable region is operably linked to an immunoglobulin heavy chain constant region, and/or (ii) an insertion of a human immunoglobulin light chain variable region that includes one or more human V_{L} segments and one or more human J_{L} segments, which human immunoglobulin light chain variable region is operably linked to an immunoglobulin light chain constant region.

In some embodiments, a non-human animal made, generated, produced, obtained or obtainable from a method as described herein is provided.

In some embodiments, a method of producing an antibody in a non-human animal is provided, the method comprising the steps of (a) immunizing a non-human animal with an antigen, which non-human animal has a genome comprising a mutant Kynu gene that encodes a Kynu polypeptide having a D93E substitution; (b) maintaining the non-human animal under conditions sufficient that the non-human animal produces an immune response to the antigen; and (c) recovering an antibody from the non-human animal, or a non-human cell, that binds the antigen.

In some embodiments of a method of producing an antibody in a non-human animal, a non-human cell is a B cell or a hybridoma. In some embodiments of a method of producing an antibody in a non-human animal, the antibody of (c) comprises human immunoglobulin heavy and/or light chain variable domains and rodent constant domains.

In some embodiments, an antigen is or comprises HIV or a fragment thereof. In some certain embodiments, an antigen is or comprises an HIV envelope protein (or polypeptide) or a fragment thereof. In some embodiments, an antigen is or comprises HIV-1 gp41 or a fragment thereof.

In some embodiments, an antigen is or comprises a peptide of the membrane proximal external region (NITER) of HIV-1 gp41 (SEQ ID NO:43); in some certain embodiments, an antigen is or comprises ELLELDKWAS (SEQ ID NO:40). In some embodiments, an antigen is or comprises QQEKNEQELLELDKWASLWN (SEQ ID NO:33). In some embodiments, an antigen is or comprises NEQELLELDKWASLWNWFNITNWLWYIK (SEQ ID NO:34).

In some embodiments, a non-human animal is provided whose genome comprises (i) a mutant Kynu gene, which mutant Kynu gene comprises one or more point mutations in exon three and encodes a Kynu polypeptide having a D93E substitution; (ii) an insertion of a human immunoglobulin heavy chain variable region that includes one or more human V_{H} segments, one or more human D_{H} segments and one or more human J_{H} segments, which human immunoglobulin heavy chain variable region is operably linked to an endogenous non-human immunoglobulin heavy chain constant region; and (ii) an insertion of a human immunoglobulin light chain variable region that includes one or more human V_{L} segments and one or more human J_{L} segments, which human immunoglobulin light chain variable region is operably linked to an endogenous non-human immunoglobulin light chain constant region.

In some embodiments, a method of producing an antibody in a non-human animal is provided, the method comprising the steps of (a) immunizing a non-human animal with the membrane proximal external region (MPER) of HIV-1 gp4, in whole or in part, which non-human animal has a genome comprising (i) a mutant Kynu gene that includes one or more point mutations in exon three and encodes a Kynu polypeptide having a D93E substitution; (ii) an insertion of a human immunoglobulin heavy chain variable region that includes one or more human V_{H} segments, one or more human D_{H} segments and one or more human J_{H} segments, which human immunoglobulin heavy chain variable region is operably linked to an endogenous non-human immunoglobulin heavy chain constant region; and (ii) an insertion of a human immunoglobulin light chain variable region that includes one or more human V_{L} segments and one or more human J_{L} segments, which human immunoglobulin light chain variable region is operably linked to an endogenous non-human immunoglobulin light chain constant region; (b) maintaining the non-human animal under conditions sufficient that the non-human animal produces an immune response to the MPER of HIV-1 gp41, in whole or in part; and (c) recovering an antibody from the non-human animal, or a non-human cell, that binds the MPER of HIV-1 gp41; wherein the antibody comprises immunoglobulin heavy chains that include human V_{H} domains linked to non-human C_{H} domains, and immunoglobulin light chains that include human Vκ domains linked to non-human Cκ domains.

In some embodiments of a method of producing an antibody in a non-human animal, a non-human animal is immunized with a peptide having the sequence ELLELDKWAS (SEQ ID NO:40). In some embodiments of a method of producing an antibody in a non-human animal, a non-human animal is immunized with a peptide having the sequence QQEKNEQELLELDKWASLWN (SEQ ID NO:33). In some embodiments of a method of producing an antibody in a non-human animal, a non-human animal is immunized with a peptide having the sequence NEQELLELDKWASLWNWFNITNWLWYIK (SEQ ID NO:34).

In some embodiments, a non-human animal HIV model is provided, which non-human animal expresses a Kynu polypeptide having a D93E substitution.

In some embodiments, a non-human animal HIV model is provided, which non-human animal has a genome comprising a mutant Kynu gene as described herein.

In some embodiments, a non-human animal HIV model is provided, obtained by (a) providing a non-human animal whose genome comprises a mutant Kynu gene as described herein; and (b) exposing the non-human animal of (a) to HIV; thereby providing said non-human animal HIV model.

In some embodiments, a non-human animal or cell as described herein is provided for use in the manufacture and/or development of a drug for therapy or diagnosis.

In some embodiments, a non-human animal or cell as described herein is provided for use in the manufacture of a medicament for the treatment, prevention or amelioration of a disease, disorder or condition.

In some embodiments, use of a non-human animal or cell as described herein in the manufacture and/or development of a drug or vaccine for use in medicine, such as use as a medicament, is provided.

In some embodiments, use of a non-human animal or cell as described herein in the manufacture and/or development of an antibody that binds HIV (e.g., an HIV envelope or portion thereof) is provided.

In some embodiments, a disease, disorder or condition is a hypertensive-related disease, disorder or condition. In some embodiments, a disease, disorder or condition is an HIV-related disease, disorder or condition or results from HIV infection and/or transmission.

In various embodiments, a Kynu gene present in the genome of a provided non-human animal encodes a Kynu polypeptide having the sequence that appears in SEQ ID NO:8 or encodes a Kynu polypeptide that contains an H4 domain that includes the sequence that appears in SEQ ID NO:36 or SEQ ID NO:41.

In various embodiments, a Kynu polypeptide expressed by a provided non-human animal has a sequence that is substantially identical or identical to SEQ ID NO:8, or contains an H4 domain that includes the sequence that appears in SEQ ID NO:36 or SEQ ID NO:41.

In various embodiments, a non-human animal as described herein is a rodent; in some embodiments, a mouse; in some embodiments, a rat. In some embodiments, a mouse as described herein is selected from the group consisting of a 129 strain, a BALB/C strain, a C57BL/6 strain, and a mixed 129×C57BL/6 strain; in some certain embodiments, a C57BL/6 strain.

As used in this application, the terms “about” and “approximately” are used as equivalents. Any numerals used in this application with or without about/approximately are meant to cover any normal fluctuations appreciated by one of ordinary skill in the relevant art.

### BRIEF DESCRIPTION OF THE DRAWING

The Drawing included herein, which is composed of the following Figures, is for illustration purposes only and not for limitation.

FIG. 1 shows a diagram, not to scale, of the genomic organization of a non-human (e.g., mouse) kynureninase (Kynu) gene. Exons are numbered above or below each exon. Untranslated regions (open boxes) and coding sequence (striped rectangle) are also indicated.

FIG. 2A shows a diagram, not to scale, of an exemplary targeting vector for creating a deletion of a kynureninase gene in a rodent as described in Example 1. A lacZ reporter gene is inserted in operable linkage to a mouse Kynu start (ATG) codon in exon two and deletes the remaining portion of exon 2 through exon 6 of the mouse Kynu locus (39.4 kb deletion). The lacZ-SDC targeting vector contains a self-deleting drug selection cassette (e.g., a neomycin resistance gene flanked by loxP sequences; see U.S. Pat. Nos. 8,697,851, 8,518,392 and 8,354,389, all of which are incorporated herein by reference). Upon homologous recombination, the sequence contained in the targeting vector is inserted in the place of exons 2-6 of an endogenous murine Kynu locus as shown. The drug selection cassette is removed in a development-dependent manner, i.e., progeny derived from mice whose germ line cells containing a disruption in a Kynu locus as described above will shed the selectable marker from differentiated cells during development. Consecutive exons (vertical slashes) are indicated by number above and below each exon, and untranslated regions (open box) and coding sequence (striped rectangle above) are also indicated. lacZ: β-galactosidase gene; Cre: Cre recombinase gene; Neo: neomycin resistance gene.

FIG. 2B shows a diagram, not to scale, of the genomic organization of a murine Kynu gene illustrating an exemplary disruption (e.g., a 39.4 kb deletion of exons 2-6) as described in Example 1. Exons (vertical slashes) are numbered above and below each exon. Untranslated regions (open boxes), coding sequence (striped rectangle) and ATG start codon are also indicated. Approximate locations of probes (i.e., 4249mTU, 4249mTD2) employed in a screening assay described in Example 1 are indicated by thick vertical slashes.

FIG. 2C shows a diagram, not to scale, of an exemplary disrupted Kynu gene as described in Example 1. A deletion of exons 2-6 (39.4 kb deletion) of a mouse Kynu locus is shown resulting from the insertion of a lacZ reporter gene operably linked to a mouse Kynu start (ATG) codon. Remaining exons (vertical slashes) are numbered above and below each exon, and untranslated regions (open box) and remaining coding sequence (striped rectangle) are also indicated. Locations of selected nucleotide junctions are marked with a line below each junction and indicated by SEQ ID NO.

FIG. 3 shows an alignment of representative amino acid sequences of human KYNU (hKYNU, SEQ ID NO:2), mouse Kynu (mKynu, SEQ ID NO:4), rat Kynu (rKynu, SEQ ID NO:6) and mutant mouse Kynu (mutKynu, SEQ ID NO:8). The epitope bound by monoclonal antibody 2F5 (see, e.g., Yang, G. et al., 2013, J. Exp. Med. 210(2):241-56) is indicated with an open box and shows a D93E amino acid substitution in mutKynu (see Examples section). Asterisk (*) indicates identical amino acids; colon (:) indicates conservative substitutions; period (.) indicates semiconservative substitutions; blank indicates non-conservative substitutions.

FIG. 4A shows a diagram, not to scale, of an exemplary targeting vector for creating a mutant Kynu gene in a rodent (e.g., mouse) as described in Example 2. Consecutive exons (vertical slashes) are indicated by number above or below each exon (exons 11-14 are not shown, see FIG. 1). Exemplary point mutations in exon three are indicated by open and filled circles (e.g., GCC to GCT, etc.) as well as a 60 bp deletion in intron three by insertion of a selection cassette by homologous recombination. Locations of selected nucleotide junctions are marked with a line below each junction and indicated by SEQ ID NO. SDC: self-deleting cassette.

FIG. 4B shows a sequence alignment of a portion of the MPER of HIV-1 gp41, the 3′ portion of exon three of a mutant Kynu gene as described in Example 2, and the amino acid sequence encoded by the 3′ portion of exon three of a mutant Kynu gene. The epitope of monoclonal antibody (mAb) 2F5 is indicated by a box over the HIV-1 gp41 sequence. Nucleotides for the last 10 codons of exon three of a mutant Kynu gene are shown below the encoded amino acid sequence. Mutated nucleotides (nt) are indicated in bold and underlined text. Mutated amino acids (AA) are indicated in bold and italicized text. HIV-1 gp41 (SEQ ID NO:40); mutKynu AA (SEQ ID NO:41); mutKynu nt (SEQ ID NO:42).

FIG. 4C shows a diagram, not to scale, of a close up view of an exemplary targeting vector for creating a mutant Kynu gene in a rodent (e.g., mouse) as described in Example 2. Exon three (grey rectangle) and intron three (black line following, or 3′ of, grey rectangle) are shown along with an exemplary cassette containing a selection marker and recombinase gene. Integration of the cassette by homologous recombination results in a 60 bp deletion in intron three. Approximate location of a probe (i.e., 4247mTU_D93E) employed in a screening assay described in Example 2 is indicated by a thick vertical slash.

FIG. 4D shows a diagram, not to scale, of a close up view of a mutant Kynu gene in a rodent (e.g., mouse) created after recombinase-mediated excision of the cassette contained within the targeting vector described in Example 2. Exon three (grey rectangle) and intron three (black line following, or 3′ of, grey rectangle) are shown with a remaining loxP site. Location of the nucleotide junction that remained after recombinase-mediated excision of the cassette is marked with a line below the junction and indicated by SEQ ID NO:26.

### DEFINITIONS

The scope of the present invention is defined by the claims appended hereto and is not limited by particular embodiments described herein; those skilled in the art, reading the present disclosure, will be aware of various modifications that may be equivalent to such described embodiments, or otherwise within the scope of the claims.

In general, terminology used herein is in accordance with its understood meaning in the art, unless clearly indicated otherwise. Explicit definitions of certain terms are provided below; meanings of these and other terms in particular instances throughout this specification will be clear to those skilled in the art from context. Additional definitions for the following and other terms are set forth throughout the specification. References cited within this specification, or relevant portions thereof, are incorporated herein by reference.

Administration: as used herein, includes the administration of a composition to a subject or system (e.g., to a cell, organ, tissue, organism, or relevant component or set of components thereof). Those of ordinary skill will appreciate that route of administration may vary depending, for example, on the subject or system to which the composition is being administered, the nature of the composition, the purpose of the administration, etc. For example, in certain embodiments, administration to an animal subject (e.g., to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Amelioration: as used herein, includes the prevention, reduction or palliation of a state, or improvement of the state of a subject. Amelioration includes but does not require complete recovery or complete prevention of a disease, disorder or condition (e.g., radiation injury).

Approximately: as applied to one or more values of interest, includes to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: as used herein, refers to a characteristic of any agent that has activity in a biological system, in vitro or in vivo (e.g., in an organism). For instance, an agent that, when present in an organism, has a biological effect within that organism is considered to be biologically active. In particular embodiments, where a protein or polypeptide is biologically active, a portion of that protein or polypeptide that shares at least one biological activity of the protein or polypeptide is typically referred to as a “biologically active” portion.

Comparable: as used herein, refers to two or more agents, entities, situations, sets of conditions, etc. that may not be identical to one another but that are sufficiently similar to permit comparison there between so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

Conservative: as used herein, refers to instances when describing a conservative amino acid substitution, including a substitution of an amino acid residue by another amino acid residue having a side chain R group with similar chemical properties (e.g., charge or hydrophobicity). In general, a conservative amino acid substitution will not substantially change the functional properties of interest of a protein, for example, the ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, AN), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445. In some embodiments, a substitution is a moderately conservative substitution wherein the substitution has a nonnegative value in the PAM250 log-likelihood matrix.

Control: as used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild-type animal). In one experiment, a “test” (i.e., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Disruption: as used herein, refers to the result of a homologous recombination event with a DNA molecule (e.g., with an endogenous homologous sequence such as a gene or gene locus). In some embodiments, a disruption may achieve or represent an insertion, deletion, substitution, replacement, missense mutation, or a frame-shift of a DNA sequence(s), or any combination thereof. Insertions may include the insertion of entire genes or fragments of genes, e.g., exons, which may be of an origin other than the endogenous sequence (e.g., a heterologous sequence). In some embodiments, a disruption may increase expression and/or activity of a gene or gene product (e.g., of a protein encoded by a gene). In some embodiments, a disruption may decrease expression and/or activity of a gene or gene product. In some embodiments, a disruption may alter sequence of a gene or an encoded gene product (e.g., an encoded polypeptide). In some embodiments, a disruption may truncate or fragment a gene or an encoded gene product (e.g., an encoded protein). In some embodiments, a disruption may extend a gene or an encoded gene product. In some such embodiments, a disruption may achieve assembly of a fusion polypeptide. In some embodiments, a disruption may affect level, but not activity, of a gene or gene product. In some embodiments, a disruption may affect activity, but not level, of a gene or gene product. In some embodiments, a disruption may have no significant effect on level of a gene or gene product. In some embodiments, a disruption may have no significant effect on activity of a gene or gene product. In some embodiments, a disruption may have no significant effect on either level or activity of a gene or gene product.

Determining, measuring, evaluating, assessing, assaying and analyzing: are used interchangeably herein to refer to any form of measurement, and include determining if an element is present or not. These terms include both quantitative and/or qualitative determinations. Assaying may be relative or absolute. “Assaying for the presence of” can be determining the amount of something present and/or determining whether or not it is present or absent.

Endogenous locus or endogenous gene: as used herein, refers to a genetic locus found in a parent or reference organism prior to introduction of a disruption, deletion, replacement, alteration, or modification as described herein. In some embodiments, the endogenous locus has a sequence found in nature. In some embodiments, the endogenous locus is a wild-type locus. In some embodiments, the reference organism is a wild-type organism. In some embodiments, the reference organism is an engineered organism. In some embodiments, the reference organism is a laboratory-bred organism (whether wild-type or engineered).

Endogenous promoter: as used herein, refers to a promoter that is naturally associated, e.g., in a wild-type organism, with an endogenous gene.

Engineered: as used herein refers, in general, to the aspect of having been manipulated by the hand of man. For example, in some embodiments, a polynucleotide may be considered to be “engineered” when two or more sequences that are not linked together in that order in nature are manipulated by the hand of man to be directly linked to one another in the engineered polynucleotide. In some particular such embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by those in the art, progeny of an engineered polynucleotide or cell are typically still referred to as “engineered” even though the actual manipulation was performed on a prior entity. Furthermore, as will be appreciated by those skilled in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc.). Alternatively or additionally, in some embodiments, “engineering” may involve use of in vitro chemical synthesis methodologies and/or recombinant nucleic acid technologies such as, for example, nucleic acid amplification (e.g., via the polymerase chain reaction) hybridization, mutation, transformation, transfection, etc., and/or any of a variety of controlled mating methodologies. As will be appreciated by those skilled in the art, a variety of established such techniques (e.g., for recombinant DNA, oligonucleotide synthesis, and tissue culture and transformation (e.g., electroporation, lipofection, etc.) are well known in the art and described in various general and more specific references that are cited and/or discussed throughout the present specification. See e.g., Sambrook et al., Molecular Cloning: A Laboratory Manual 2^{nd} ed., Cold Spring Harbor Laboratory Press, Cold Spring Harbor, N.Y., 1989.

Gene: as used herein, refers to a DNA sequence in a chromosome that codes for a product (e.g., an RNA product and/or a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). For the purpose of clarity we note that, as used in the present application, the term “gene” generally refers to a portion of a nucleic acid that encodes a polypeptide; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid.

Heterologous: as used herein, refers to an agent or entity from a different source. For example, when used in reference to a polypeptide, gene, or gene product present in a particular cell or organism, the term clarifies that the relevant polypeptide, gene, or gene product: 1) was engineered by the hand of man; 2) was introduced into the cell or organism (or a precursor thereof) through the hand of man (e.g., via genetic engineering); and/or 3) is not naturally produced by or present in the relevant cell or organism (e.g., the relevant cell type or organism type). “Heterologous” also includes a polypeptide, gene or gene product that is normally present in a particular native cell or organism, but has been modified, for example, by mutation or placement under the control of non-naturally associated and, in some embodiments, non-endogenous regulatory elements (e.g., a promoter).

Host cell: as used herein, refers to a cell into which a nucleic acid or protein has been introduced. Persons of skill upon reading this disclosure will understand that such terms refer not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the phrase “host cell”. In some embodiments, a host cell is or comprises a prokaryotic or eukaryotic cell. In general, a host cell is any cell that is suitable for receiving and/or producing a heterologous nucleic acid or protein, regardless of the Kingdom of life to which the cell is designated. Exemplary cells include those of prokaryotes and eukaryotes (single-cell or multiple-cell), bacterial cells (e.g., strains of Escherichia coli, Bacillus spp., Streptomyces spp., etc.), mycobacteria cells, fungal cells, yeast cells (e.g., Saccharomyces cerevisiae, Schizosaccharomyces pombe, Pichia pastoris, Pichia methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6® cell). In some embodiments, a host cell is or comprises an isolated cell. In some embodiments, a host cell is part of a tissue. In some embodiments, a host cell is part of an organism.

Identity: as used herein in connection with a comparison of sequences, refers to identity as determined by a number of different algorithms known in the art that can be used to measure nucleotide and/or amino acid sequence identity. In some embodiments, identities as described herein are determined using a ClustalW v. 1.83 (slow) alignment employing an open gap penalty of 10.0, an extend gap penalty of 0.1, and using a Gonnet similarity matrix (MACVECTOR™ 10.0.2, MacVector Inc., 2008).

In vitro: as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and/or a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. In some embodiments, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when: a) by virtue of its origin or source of derivation is not associated with some or all of the components that accompany it in its native state in nature; b) it is substantially free of other polypeptides or nucleic acids of the same species from the species that produces it in nature; or c) is expressed by or is otherwise in association with components from a cell or other expression system that is not of the species that produces it in nature. Thus, for instance, in some embodiments, a polypeptide that is chemically synthesized, or is synthesized in a cellular system different from that which produces it in nature, is considered to be an “isolated” polypeptide. Alternatively or additionally, in some embodiments, a polypeptide that has been subjected to one or more purification techniques may be considered to be an “isolated” polypeptide to the extent that it has been separated from other components: a) with which it is associated in nature; and/or b) with which it was associated when initially produced.

Locus or Loci: as used herein, refers to a specific location(s) of a gene (or significant sequence), DNA sequence, polypeptide-encoding sequence, or position on a chromosome of the genome of an organism. For example, a “Kynu locus” may refer to the specific location of a Kynu gene, Kynu DNA sequence, Kynu-encoding sequence, or Kynu position on a chromosome of the genome of an organism that has been identified as to where such a sequence resides. A “Kynu locus” may comprise a regulatory element of a Kynu gene, including, but not limited to, an enhancer, a promoter, 5′ and/or 3′ UTR, or a combination thereof. Those of ordinary skill in the art will appreciate that chromosomes may, in some embodiments, contain hundreds or even thousands of genes and demonstrate physical co-localization of similar genetic loci when comparing between different species. Such genetic loci can be described as having shared synteny.

Non-human animal: as used herein, refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human animal is a mammal. In some embodiments, a non-human mammal is a primate, a goat, a sheep, a pig, a dog, a cow, or a rodent. In some embodiments, a non-human animal is a rodent such as a rat or a mouse.

Nucleic acid: as used herein, refers to any compound and/or substance that is or can be incorporated into an oligonucleotide chain. In some embodiments, a “nucleic acid” is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a “nucleic acid” is, comprises, or consists of one or more natural nucleic acid residues. In some embodiments, a “nucleic acid” is, comprises, or consists of one or more nucleic acid analogs. In some embodiments, a nucleic acid analog differs from a “nucleic acid” in that it does not utilize a phosphodiester backbone. For example, in some embodiments, a “nucleic acid” is, comprises, or consists of one or more “peptide nucleic acids”, which are known in the art and have peptide bonds instead of phosphodiester bonds in the backbone, are considered within the scope of the present invention. Alternatively or additionally, in some embodiments, a “nucleic acid” has one or more phosphorothioate and/or 5′-N-phosphoramidite linkages rather than phosphodiester bonds. In some embodiments, a “nucleic acid” is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a “nucleic acid” is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a “nucleic acid” comprises one or more modified sugars (e.g., 2′-fluororibose, ribose, 2′-deoxyribose, arabinose, and hexose) as compared with those in natural nucleic acids. In some embodiments, a “nucleic acid” has a nucleotide sequence that encodes a functional gene product such as an RNA or protein. In some embodiments, a “nucleic acid” includes one or more introns. In some embodiments, a “nucleic acid” includes one or more exons. In some embodiments, a “nucleic acid” is prepared by one or more of isolation from a natural source, enzymatic synthesis by polymerization based on a complementary template (in vivo or in vitro), reproduction in a recombinant cell or system, and chemical synthesis. In some embodiments, a “nucleic acid” is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a “nucleic acid” is single stranded; in some embodiments, a “nucleic acid” is double stranded. In some embodiments, a “nucleic acid” has a nucleotide sequence comprising at least one element that encodes, or is the complement of a sequence that encodes, a polypeptide. In some embodiments, a “nucleic acid” has enzymatic activity.

Operably linked: as used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest. The term “expression control sequence” includes polynucleotide sequences, which are necessary to affect the expression and processing of coding sequences to which they are ligated. “Expression control sequences” include: appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing, and can also include additional components whose presence is advantageous, for example, leader sequences and fusion partner sequences.

Physiological conditions: as used herein, refers to its art-understood meaning referencing conditions under which cells or organisms live and/or reproduce. In some embodiments, the term includes conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Polypeptide: as used herein, refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. In some embodiments, a polypeptide has an amino acid sequence that is a variant in that it contains one or more amino acid substitutions as compared to a parent or reference polypeptide.

Recombinant: as used herein, is intended to refer to polypeptides (e.g., Kynu polypeptides as described herein) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom, H. R., 1997, TIB Tech. 15:62-70; Azzazy, H. and W. E. Highsmith, 2002, Clin. Biochem. 35:425-45; Gavilondo, J. V. and J. W. Larrick, 2002, BioTechniques 29:128-45; Hoogenboom H., and P. Chames, 2000, Immunol. Today 21:371-8), antibodies isolated from an animal (e.g., a mouse) that is transgenic for human immunoglobulin genes (see e.g., Taylor, L. D. et al., 1992, Nucl. Acids Res. 20:6287-95; Kellermann, S-A. and L. L. Green, 2002, Curr. Opin. Biotechnol. 13:593-7; Little, M. et al., 2000, Immunol. Today 21:364-70; Murphy, A. J. et al., 2014, Proc. Natl. Acad. Sci. U.S.A. 111(14):5153-8) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements result from mutagenesis (e.g., in vivo or in vitro) of a known sequence element, e.g., from a natural or synthetic source. For example, in some embodiments, a recombinant polypeptide is comprised of sequences found in the genome of a source organism of interest (e.g., human, mouse, etc.). In some embodiments, a recombinant polypeptide has an amino acid sequence that resulted from mutagenesis (e.g., in vitro or in vivo, for example, in a non-human animal), so that the amino acid sequences of the recombinant polypeptides are sequences that, while originating from and related to polypeptides sequences, may not naturally exist within the genome of a non-human animal in vivo.

Reference: as used herein, refers to a standard or control agent, animal, cohort, individual, population, sample, sequence or value against which an agent, animal, cohort, individual, population, sample, sequence or value of interest is compared. In some embodiments, a reference agent, animal, cohort, individual, population, sample, sequence or value is tested and/or determined substantially simultaneously with the testing or determination of an agent, animal, cohort, individual, population, sample, sequence or value of interest. In some embodiments, a reference agent, animal, cohort, individual, population, sample, sequence or value is a historical reference, optionally embodied in a tangible medium. In some embodiments, a reference may refer to a control. A “reference” also includes a “reference animal”. A “reference animal” may have a modification as described herein, a modification that is different as described herein or no modification (i.e., a wild-type animal). Typically, as would be understood by those skilled in the art, a reference agent, animal, cohort, individual, population, sample, sequence or value is determined or characterized under conditions comparable to those utilized to determine or characterize an agent, animal (e.g., a mammal), cohort, individual, population, sample, sequence or value of interest.

Replacement: as used herein, refers to a process through which a “replaced” nucleic acid sequence (e.g., a gene) found in a host locus (e.g., in a genome) is removed from that locus, and a different, “replacement” nucleic acid is located in its place. In some embodiments, the replaced nucleic acid sequence and the replacement nucleic acid sequences are comparable to one another in that, for example, they are homologous to one another and/or contain corresponding elements (e.g., protein-coding elements, regulatory elements, etc.). In some embodiments, a replaced nucleic acid sequence includes one or more of a promoter, an enhancer, a splice donor site, a splice acceptor site, an intron, an exon, an untranslated region (UTR); in some embodiments, a replacement nucleic acid sequence includes one or more coding sequences. In some embodiments, a replacement nucleic acid sequence is a homolog or variant (e.g., mutant) of the replaced nucleic acid sequence. In some embodiments, a replacement nucleic acid sequence is an ortholog or homolog of the replaced sequence. In some embodiments, a replacement nucleic acid sequence is or comprises a human nucleic acid sequence. In some embodiments, including where the replacement nucleic acid sequence is or comprises a human nucleic acid sequence, the replaced nucleic acid sequence is or comprises a rodent sequence (e.g., a mouse or rat sequence). In some embodiments, a replacement nucleic acid sequence is a variant or mutant (i.e., a sequence that contains one or more sequence differences, e.g., substitutions, as compared to the replaced sequence) of the replaced sequence. The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, the replacement is a substitution of an endogenous sequence with a heterologous sequence that results in the production of a gene product from the nucleic acid sequence so placed (comprising the heterologous sequence), but not expression of the endogenous sequence; the replacement is of an endogenous genomic sequence with a nucleic acid sequence that encodes a polypeptide that has a similar function as a polypeptide encoded by the endogenous sequence (e.g., the endogenous genomic sequence encodes a Kynu polypeptide, and the DNA fragment encodes one or more variant Kynu polypeptides, in whole or in part). In various embodiments, an endogenous gene or fragment thereof is replaced with a corresponding mutant gene or fragment thereof. A corresponding mutant gene or fragment thereof is a mutant gene or fragment thereof that is substantially similar or the same in structure and/or function as the endogenous gene or fragment thereof that is replaced.

Substantially: as used herein, refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: as used herein, refers to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, S. F. et al., 1990, J. Mol. Biol., 215(3): 403-10; Altschul, S. F. et al., 1996, Meth. Enzymol. 266:460-80; Altschul, S. F. et al., 1997, Nucleic Acids Res., 25:3389-402; Baxevanis, A. D. and B. F. F. Ouellette (eds.) Bioinformatics: A Practical Guide to the Analysis of Genes and Proteins, Wiley, 1998; and Misener et al. (eds.) Bioinformatics Methods and Protocols, Methods in Molecular Biology, Vol. 132, Humana Press, 1998. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 9, 10, 11, 12, 13, 14, 15, 16, 17 or more residues. In some embodiments, the relevant stretch includes contiguous residues along a complete sequence. In some embodiments, the relevant stretch includes discontinuous residues along a complete sequence, for example, noncontiguous residues brought together by the folded conformation of a polypeptide or a portion thereof. In some embodiments, the relevant stretch is at least 10, 15, 20, 25, 30, 35, 40, 45, 50, or more residues.

Substantial identity: as used herein, refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, S. F. et al., 1990, J. Mol. Biol., 215(3): 403-10; Altschul, S. F. et al., 1996, Meth. Enzymol. 266:460-80; Altschul, S. F. et al., 1997, Nucleic Acids Res., 25:3389-402; Baxevanis, A. D. and B. F. F. Ouellette (eds.) Bioinformatics: A Practical Guide to the Analysis of Genes and Proteins, Wiley, 1998; and Misener et al. (eds.) Bioinformatics Methods and Protocols, Methods in Molecular Biology, Vol. 132, Humana Press, 1998. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, or more residues.

Targeting vector or targeting construct: as used herein, refers to a polynucleotide molecule that comprises a targeting region. A targeting region comprises a sequence that is identical or substantially identical to a sequence in a target cell, tissue or animal and provides for integration of the targeting construct (and/or a sequence contained therein) into a position within the genome of the cell, tissue or animal via homologous recombination. Targeting regions that target into a position of the cell, tissue or animal via recombinase-mediated cassette exchange using site-specific recombinase recognition sites (e.g., loxP or Frt sites) are also included. In some embodiments, a targeting construct as described herein further comprises a nucleic acid sequence or gene (e.g., a reporter gene, homologous gene, heterologous gene, or mutant gene) of particular interest, a selectable marker, control and/or regulatory sequences, and other nucleic acid sequences that encode a recombinase or recombinogenic polypeptide. In some embodiments, a targeting construct may comprise a gene of interest in whole or in part, wherein the gene of interest encodes a polypeptide, in whole or in part, that has a similar function as a protein encoded by an endogenous sequence. In some embodiments, a targeting construct may comprises a mutant gene of interest, in whole or in part, wherein the mutant gene of interest encodes a variant polypeptide, in whole or in part, that has a similar function as a polypeptide encoded by an endogenous sequence. In some embodiments, a targeting construct may comprise a reporter gene, in whole or in part, wherein the reporter gene encodes a polypeptide that is easily identified and/or measured using techniques known in the art.

Variant: as used herein, refers to an entity that shows significant structural identity with a reference entity, but differs structurally from the reference entity in the presence or level of one or more chemical moieties as compared with the reference entity. In some embodiments, a “variant” also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A “variant”, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs. double, E vs. Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a “variant polypeptide” may differ from a reference polypeptide as a result of one or more differences in amino acid sequence and/or one or more differences in chemical moieties (e.g., carbohydrates, lipids, etc.) covalently attached to the polypeptide backbone. In some embodiments, a “variant polypeptide” shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a “variant polypeptide” does not share at least one characteristic sequence element with a reference polypeptide. In some embodiments, the reference polypeptide has one or more biological activities. In some embodiments, a “variant polypeptide” shares one or more of the biological activities of the reference polypeptide. In some embodiments, a “variant polypeptide” lacks one or more of the biological activities of the reference polypeptide. In some embodiments, a “variant polypeptide” shows a reduced level of one or more biological activities as compared with the reference polypeptide. In some embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, or 2% of the residues in the variant are substituted as compared with the parent. In some embodiments, a “variant” has 10, 9, 8, 7, 6, 5, 4, 3, 2, or 1 substituted residue(s) as compared with a parent. Often, a “variant” has a very small number (e.g., fewer than 5, 4, 3, 2, or 1) number of substituted functional residues (i.e., residues that participate in a particular biological activity). Furthermore, a “variant” typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, a parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

Vector: as used herein, refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operably linked genes are referred to herein as “expression vectors.”

Wild-type: as used herein, refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

### DETAILED DESCRIPTION OF CERTAIN EMBODIMENTS

Non-human animals are provided having disruption or mutation(s) in the genetic material encoding a kynureninase (Kynu) polypeptide. In particular, non-human animals having a deletion, in whole or in part, of the coding sequence of a Kynu gene that results in the elimination of a Kynu polypeptide from the non-human animal are provided. Also provided are non-human animals having one or more mutations in a coding sequence of a Kynu gene that results in an encoded gene product that includes an amino acid substitution resulting in the elimination of a shared epitope present in human immunodeficiency virus (HIV). Described herein are non-human animals having one or more point mutations in a Kynu gene that results in a conservative amino acid substitution (e.g., substitution of aspartic acid [Asp, D] with glutamic acid [Glu, E]) in the encoded Kynu polypeptide. Such an amino acid substitution, as described herein, results in the elimination of a shared epitope present in an endogenous Kynu polypeptide expressed by a non-human animal and the membrane proximal extended region (MPER) of HIV-1 gp41. Therefore, provided non-human animals are particularly useful for the development and identification of therapeutic candidates for the treatment and/or amelioration of HIV infection and/or transmission that are otherwise not obtainable with wild-type non-human animals that express a Kynu polypeptide containing such an epitope due to self-tolerance mechanisms. In particular, non-human animals described herein encompass the introduction of one or more point mutations (e.g., 1, 2, 3, 4, 5, etc.) into the coding sequence of an endogenous Kynu gene resulting in the expression of a Kynu polypeptide (e.g., a variant Kynu polypeptide) that retains the function of a wild-type Kynu polypeptide yet lacks an epitope that is also present in the NITER of HIV-1 gp41. Such non-human animals provide a source of cells for identifying neutralizing antibodies for the treatment and/or amelioration of HIV infection and/or transmission. Further, such non-human animals provide the capacity for a useful animal model system for the development of therapeutics for the treatment of HIV infection, transmission and/or diseases, disorders and conditions related thereto.

In some embodiments, non-human animals described herein are heterozygous for a disruption or mutation(s) in a Kynu gene as described herein. In some embodiments, non-human animals described herein are homozygous for a disruption or mutation(s) in a Kynu gene as described herein. In some embodiments, non-human animals as described herein comprise a reporter gene, in whole or in part, wherein said reporter gene is operably linked to a Kynu promoter. In some embodiments, Kynu promoters include endogenous Kynu promoters.

In some embodiments, Kynu polypeptides expressed by non-human animals described herein comprise an H4 domain sequence that includes the amino acid sequence ELEKWA (SEQ ID NO:36). In some embodiments, Kynu polypeptides expressed by non-human animals described herein comprise an H4 domain sequence that appears in a wild-type rodent Kynu polypeptide and further includes an amino acid substitution at residue 93 (e.g., an amino acid substitution with an amino acid other than an amino acid that appears in a wild-type rodent Kynu polypeptide). In some certain embodiments, Kynu polypeptides expressed by non-human animals described herein comprise an H4 domain sequence that appears in a wild-type rodent Kynu polypeptide and further includes a D93E substitution. Thus, such Kynu polypeptides may, in some embodiments, be characterized or referred to as variant Kynu polypeptides.

In some embodiments, non-human animals as described herein comprise a deletion, disruption or otherwise non-functional endogenous Kynu gene and further comprise genetic material from a heterologous species (e.g., a human). In some embodiments, non-human animals as described herein comprise a mutant human Kynu gene, wherein the mutant human Kynu gene encodes a human Kynu polypeptide that includes a D93E substitution. In some certain embodiments, non-human animals as described herein comprise a mutant human Kynu gene that is randomly inserted into the genome of the non-human animal such that a human Kynu polypeptide is expressed that includes a D93E substitution.

Various aspects of the invention are described in detail in the following sections. The use of sections is not meant to limit the invention. Each section can apply to any aspect of the invention. In this application, the use of “or” means “and/or” unless stated otherwise.

### Autoimmunity

B cell receptors are assembled through a series of recombination events from ordered arrangement of gene segments (e.g., V, D and J). This assembly of gene segments is known to be imprecise and generates receptors having affinity for various antigens, including self-antigens. Despite this capacity to generate B cell receptors that bind self-molecules, the immune system is equipped with several self-tolerance mechanisms to avoid development and expansion of such auto-reactive B cell receptors and discriminate self from non-self thereby preventing autoimmunity (see, e.g., Shlomchik, M. J., 2008, Immunity 28:18-28; Kumar, K. R. and C. Mohan, 2008, 40(3):208-23). When such self-tolerance mechanisms breakdown or are otherwise functioning improperly, autoimmunity results and manifests itself in a variety of disorders depending on the immune cell (e.g., B or T cell) and antigen involved. For example, the aberrant expansion of auto-reactive antibodies that bind thyroid stimulating hormone receptor result in the overproduction of thyroid hormones thereby leading to Grave's disease. Also, generation and expansion of auto-reactive antibodies that bind to self-molecules such as, for example, DNA, chromatin, and ribonucleoproteins results in severe inflammatory conditions such as glomerulonephritis and vasculitis thereby leading to a condition referred to as systemic lupus erythematosus (SLE). Mechanisms employed by the immune system to protect against a breakdown in self-tolerance include, for example, deletion and receptor editing of auto-reactive B cells in the bone marrow and thymus, inactivation (or anergy) via lack of or weak signaling of co-stimulatory molecules in peripheral organs, and physical separation of self-molecules from lymphoid tissue. Self-tolerance mechanisms and autoimmunity are discussed in detail in Murphy, K., 2012, Janeway's Immunobiology: 8^{th} ed. Chapters 8 and 15: Garland Sciences, pp. 275-333, 611-668; incorporated herein by reference.

Self-tolerance mechanisms, however, also come with negative consequences. For example, through the manipulation of various molecules, cancer cells are able to induce tolerance mechanisms and evade a host's immune system as a result of inhibition and/or down-regulation of anti-tumor immunity. Also, viral pathogens have been found to effectively infect a host and evade elimination by suppression of antibody responses (see, e.g., Yamada, D. H. et al., 2015, Immunity 42(2):379-90). In particular, several reports have demonstrated that human immunodeficiency virus (HIV) is resistant to immune responses due to induction of self-tolerance mechanisms that suppress development of broadly neutralizing antibodies until it is too late to positively change the course of disease (see, e.g., Verkoczy, L. and M. Diaz, 2014, Curr. Opin. HIV AIDS 9(3):224-34; Haynes, B. F. et al., 2011, Trends Mol. Med. 17(2):108-16; Verkoczy, L. et al., 2011, Curr. Opin. Immunol. 23:383-90; Haynes, B. F. et al., 2005, Science 308:1906-8).

HIV is an integrating, enveloped lentivirus (a subgroup of retroviruses) that enters cells by membrane fusion (Harrison, S.C., 2005, Adv. Virus. Res. 64:231-61). The structure, genome and lifecycle of HIV have been well documented. The HIV genome is surrounded by a viral envelope, which includes a lipid bilayer and other proteins taken from a host cell as well as the HIV envelope protein consisting of a cap that includes glycoproteins 120 and 41 (gp120 and gp41). HIV infects important immune cells, most notably, CD4⁺ T cells, and results in immune dysfunction and loss of cell-mediated immunity due, in part, to the decrease of CD4⁺ T cells. Although initial B cell responses are detectable soon after HIV infection, they remain ineffective at controlling plasma HIV levels (see, e.g., Haynes, B. F. et al., 2011, Trends Mol. Med. 17(2):108-16; Bar, K. J. et al., 2010, AIDS Res. Human Retroviruses 26:A-12; Tomaras, G. D. et al., 2008, J. Virol. 82:12449-63). Despite the observed ineffective immune response to HIV, six neutralizing antibodies (2G12, b12, 447-52D, 2F5, 4E10, Z13) that bind gp120 or gp41 have been identified from patients (Gorny, M. K. et al., 1993, J. Immunol. 150(2):635-43; Muster, T. et al., 1993, J. Virol. 67:6642-7; Buchacher, A. et al., 1994, AIDS Res. Human Retroviruses 10:359-69; Burton, D. R. et al., 1994, Science 266:1024-7; Muster, T. et al., 1994, J. Virol. 68:4031-4; Purtscher, M. et al., 1994, AIDS Res. Hum. Retroviruses 10:1651-8; Roben, P. et al., 1994, J. Virol. 68:4821-8; Parren, P. W. et al., 1995, AIDS 9:F1-F6; Trkola, A. et al., 1995, J. Virol. 69:6609-17; Trkola, A. et al., 1996, J. Virol. 70:1100-8; Stiegler, G. et al., 2001, AIDS Res. Hum. Retroviruses 17:1757-65; Zwick, M. B. et al., 2001, J. Virol. 75:10892-905; Stiegler, G. and H. Katinger, 2003, J. Antimicrobiol. Chemother. 51:757-9; Ofek, G. et al., 2004, J. Virol. 19:10724-37; Cardoso, R. M. F. et al., 2005, Immunity 22:163-73). Among these identified neutralizing antibodies, monoclonal antibodies 2F5 and 4E10, which bind an epitope in the membrane proximal extended region (MPER, ELLELDKWASLWNWFDITNWLWYIK; SEQ ID NO:43) of gp41 of HIV type 1 (HIV-1), have been reported to also bind self-antigens (Haynes, B. F. et al., 2005, Science 308:1906-8; Verkoczy, L. et al., 2010, Proc. Nat. Acad. Sci. U.S.A. 107(1):181-6; Verkoczy, L. et al., 2011, J. Immunol. 187:3785-97). Indeed, the MPER remains a target for HIV-1 vaccine design (for a review see, e.g., Montero, M. et al., 2008, Microbiol. Mol. Biol. Rev. 72(1):54-84).

Kynureninase (Kynu) has recently been identified as a self-antigen that contains a domain (H4 domain) that includes the complete MPER epitope bound by monoclonal antibody 2F5 (Yang, G. et al., 2013, J. Exp. Med. 210(2):241-56). Kynu is a pyridoxal-5′-phosphate (pyridoxal-P) dependent enzyme that catalyzes the cleavage of L-kynurenine and L-3-hydroxykynurenine into anthranilic and 3-hydroxyanthranilic acids, respectively, and is involved in the biosynthesis of NAD cofactors from tryptophan through the kynurenine pathway. Alternative splicing results in multiple transcript variants (see below). Some reports have linked Kynu activity with hypertension (Kwok, J. B. et al., 2002, J. Biol. Chem. 277(39):35779-82; Mizutani, K. et al., 2002, Hypertens. Res. 25(1):135-40; Zhang, Y. et al., 2011, Circ. Cardiovasc. Genet. 4:687-94). The identification of shared epitopes between existing neutralizing antibodies against HIV and self-antigens has provided the insight that B cells producing such antibodies are likely deleted from the immunological repertoire due to their autoreactivity and, thus, effective antibody responses to HIV are likely drastically impaired or non-existent in patients.

Production of antibodies that bind self-antigens has been described (see, e.g., U.S. Pat. Nos. 5,885,793, 6,521,404, 6,544,731, 6,555,313, 6,582,915, 6,593,081, 7,119,248, 7,195,866, 7,459,158, 8,013,208, 8,025,873, 8,293,701, 8,389,793, 8,465,745 and 8,563,003). In particular, methods for obtaining monoclonal antibodies that bind self-antigens or homologs thereof in non-human animals have been accomplished through the knockout of genes in non-human animals that share significant homology and/or are highly conserved with their human counterpart genes (see U.S. Pat. No. 7,119,248). Immunization of non-human animals (e.g., rodents) with human antigens that are highly similar, or “homologous”, yields weak or non-existent antibody responses and, therefore, makes it problematic to obtain antibodies with binding directed to such human antigens. The present invention is based on the insight that the presence of such shared epitopes between endogenous polypeptides and a foreign pathogen such as a virus makes mounting an effective immune response in a non-human animal that neutralizes such foreign entities problematic because immunological tolerance depletes and/or deletes B cells that express neutralizing antibodies against such foreign entities. Thus, the present invention is based on the recognition that improved in vivo systems for generating and developing therapeutic antibodies that recognize epitopes in a non-human animal that are shared with foreign entities (e.g., a virus) can be generated by elimination of such shared epitopes present in endogenous gene products in a non-human animal such as a rodent (e.g., a mouse) without eliminating the function of such gene products. The present disclosure demonstrates, among other things, exemplary strategies of eliminating epitopes from an endogenous gene product in a non-human animal that are present in an antigen that is not a homolog of the endogenous gene product.

As described herein, the present disclosure specifically describes strategies for elimination of a shared epitope present in an endogenous Kynu polypeptide of a rodent and HIV so that anti-HIV antibodies can be produced in the rodent. In particular, the present disclosure specifically describes methods in which genetic material encoding a rodent Kynu polypeptide is engineered to eliminate epitopes present in Kynu polypeptides that are also present in gp41 of HIV-1. In one strategy, a rodent is genetically engineered to delete, in whole or in part, the genetic material that encodes an endogenous Kynu polypeptide that contains an epitope that is also present in the MPER of HIV-1 gp41. In another strategy, a rodent is genetically engineered to alter the genetic material that encodes an endogenous Kynu polypeptide so that the resulting Kynu polypeptide expressed by the rodent is a Kynu polypeptide that lacks a shared epitope (i.e., a variant Kynu polypeptide) present in the MPER of HIV-1 gp41. It is contemplated that such variant Kynu polypeptides expressed by rodents described herein are structurally and functionally equivalent to wild-type Kynu polypeptides.

Without wishing to be bound by any particular theory, the strategies described herein can be employed to eliminate an epitope present in any other endogenous gene product of a non-human animal such as a rodent, or combination of epitopes present in one or more endogenous gene products, which epitope is also present in HIV (e.g., in an HIV envelope protein) as desired. Examples of such endogenous gene products have been described in Yang, G. et al. (2013, supra) and include apoptosis-inducing factor 1 mitochondrial precursor (AIFM1), fatty aldehyde dehydrogenase (ALDH3A2), ATPase family AAA domain-containing protein 3A (ATAD3A), erlin-2 (ERLN2), emerin (EMD), glyceraldehyde-3-phosphate dehydrogenase (GAPDH), 60kD heat shock protein mitochondrial precursor (HSP60), tubulin □-1B chain (K-ALPHA-1), kynureninase (KYNU), dolichyldiphosphooligosaccharide-protein glycosyltransferase 48 kD subunit precursor (OST48), prohibitin (PHB), 60S ribosomal protein L4 (RPL4), 60S ribosomal protein L7 (RPL7), splicing factor 3B subunit 3 (SF3B3), mitochondrial 2-oxoglutarate/malate carrier protein (SLC25A11), heterogeneous nuclear ribonucleoprotein Q (SYNCRIP), tubulin □-4A chain (TUBB4) and elongation factor Tu mitochondrial precursor (TUFM). Thus, the present invention provides, among other things, the creation of an improved in vivo system for the development of antibodies and/or antibody-based therapeutics for the treatment and/or amelioration of HIV infection and transmission.

### Exemplified Self-Antigen Sequences

Exemplary human and rodent (e.g., rat and mouse) Kynu sequences are set forth below. For mRNA sequences, bold font within parentheses indicates coding sequence, and consecutive exons, where indicated, are separated by alternating underlined text.

Human KYNU transcript variants are known in the art. For example, one human KYNU transcript variant (variant 2) differs in the 5′ untranslated region, 3′ untranslated region and coding region as compared to variant 3. The resulting isoform (isoform b) is shorter (307 amino acids) and has a distinct C-terminus as compared to isoform a. The mRNA and amino acid sequences of this variant can be found at NCBI reference numbers NM_001032998.1 and NP_001028170.1, respectively, and are incorporated herein by reference. Another human KYNU transcript variant (variant 3) represents the longest transcript variant and encodes isoform a (as does variant 1, see below). The mRNA and amino acid sequences of this variant can be found at NCBI reference numbers NM_001199241.1 and NP_001186170.1, respectively, and are incorporated herein by reference.

Mouse Kynu transcripts are also known in the art. For example, one mouse Kynu transcript variant (variant 2) contains a 3′ terminal exon that extends past a splice site used in variant 1 and results in a novel 3′ coding region and 3′ untranslated region as compared to variant 1. This variant (variant 2) encodes isoform 2, which is shorter (428 amino acids) and has a distinct C-terminus as compared to isoform 1. The mRNA and amino acid sequences of this variant can be found at NCBI reference numbers NM_001289593.1 and NP_001276522.1, respectively, and are incorporated herein by reference. Another mouse Kynu transcript variant (variant 3) includes an alternate 3′ terminal exon as compared to variant 1. This variant (variant 3) encodes isoform 3, which is shorter (324 amino acids) and has a distinct C-terminus as compared to isoform 1. The mRNA and amino acid sequences of this variant can be found at NCBI reference numbers NM_001289594.1 and NP_001276523.1, respectively, and are incorporated herein by reference.

Homo sapiens KYNU transcript variant 1 mRNA (NCBI reference sequence
NM_003937.2; SEQ ID NO: 1):
GCAGTTCTTTGAATTTCTCACCCTAAGATCTGGCCTGTACATTTTCAAGGAATTCTTGAG
AGGTTCTTGGAGAGATTCTGGGAGCCAAACACTCCATTGGGATCCTAGCTGTTTTAGAG
AACAACTTGTA(ATGGAGCCTTCATCTCTTGAGCTGCCGGCTGACACAGTGCAGCG
CATTGCGGCTGAACTCAAATGCCACCCAACGGATGAGAGGGTGGCTCTCCACCTA
GATGAGGAAGATAAGCTGAGGCACTTCAGGGAGTGCTTTTATATTCCCAAAATAC
AGGATCTGCCTCCAGTTGATTTATCATTAGTGAATAAAGATGAAAATGCCATCTAT
TTCTTGGGAAATTCTCTTGGCCTTCAACCAAAAATGGTTAAAACATATCTTGAAGA
AGAACTAGATAAGTGGGCCAAAATAGCAGCCTATGGTCATGAAGTGGGGAAGCGT
CCTTGGATTACAGGAGATGAGAGTATTGTAGGCCTTATGAAGGACATTGTAGGAG
CCAATGAGAAAGAAATAGCCCTAATGAATGCTTTGACTGTAAATTTACATCTTCTA
ATGTTATCATTTTTTAAGCCTACGCCAAAACGATATAAAATTCTTCTAGAAGCCAA
AGCCTTCCCTTCTGATCATTATGCTATTGAGTCACAACTACAACTTCACGGACTTA
ACATTGAAGAAAGTATGCGGATGATAAAGCCAAGAGAGGGGGAAGAAACCTTAA
GAATAGAGGATATCCTTGAAGTAATTGAGAAGGAAGGAGACTCAATTGCAGTGAT
CCTGTTCAGTGGGGTGCATTTTTACACTGGACAGCACTTTAATATTCCTGCCATCA
CAAAAGCTGGACAAGCGAAGGGTTGTTATGTTGGCTTTGATCTAGCACATGCAGT
TGGAAATGTTGAACTCTACTTACATGACTGGGGAGTTGATTTTGCCTGCTGGTGTT
CCTACAAGTATTTAAATGCAGGAGCAGGAGGAATTGCTGGTGCCTTCATTCATGA
AAAGCATGCCCATACGATTAAACCTGCATTAGTGGGATGGTTTGGCCATGAACTC
AGCACCAGATTTAAGATGGATAACAAACTGCAGTTAATCCCTGGGGTCTGTGGAT
TCCGAATTTCAAATCCTCCCATTTTGTTGGTCTGTTCCTTGCATGCTAGTTTAGAG
ATCTTTAAGCAAGCGACAATGAAGGCATTGCGGAAAAAATCTGTTTTGCTAACTG
GCTATCTGGAATACCTGATCAAGCATAACTATGGCAAAGATAAAGCAGCAACCAA
GAAACCAGTTGTGAACATAATTACTCCGTCTCATGTAGAGGAGCGGGGGTGCCAG
CTAACAATAACATTTTCTGTTCCAAACAAAGATGTTTTCCAAGAACTAGAAAAAAG
AGGAGTGGTTTGTGACAAGCGGAATCCAAATGGCATTCGAGTGGCTCCAGTTCCT
CTCTATAATTCTTTCCATGATGTTTATAAATTTACCAATCTGCTCACTTCTATACTT
GACTCTGCAGAAACAAAAAATTAG)CAGTGTTTTCTAGAACAACTTAAGCAAATTATA
CTGAAAGCTGCTGTGGTTATTTCAGTATTATTCGATTTTTAATTATTGAAAGTATGTCAC
CATTGACCACATGTAACTAACAATAAATAATATACCTTACAGAAAATCTGAAAAAAAA
AAAAAAAAA
Homo sapiens KYNU isoform a, 465 amino acids encoded by transcript variant 1 (NCBI
reference sequence NP_003928.1; SEQ ID NO: 2):
MEPSSLELPADTVQRIAAELKCHPTDERVALHLDEEDKLRHFRECFYIPKIQDLPPVDLSLV
NKDENAIYFLGNSLGLQPKMVKTYLEEELDKWAKIAAYGHEVGKRPWITGDESIVGLMKD
IVGANEKEIALMNALTVNLHLLMLSFFKPTPKRYKILLEAKAFPSDHYAIESQLQLHGLNIE
ESMRMIKPREGEETLRIEDILEVIEKEGDSIAVILFSGVHFYTGQHFNIPAITKAGQAKGCYV
GFDLAHAVGNVELYLHDWGVDFACWCSYKYLNAGAGGIAGAFIHEKHAHTIKPALVGWF
GHELSTRFKMDNKLQLIPGVCGFRISNPPILLVCSLHASLEIFKQATMKALRKKSVLLTGYL
EYLIKHNYGKDKAATKKPVVNIITPSHVEERGCQLTITFSVPNKDVFQELEKRGVVCDKRN
PNGIRVAPVPLYNSFHDVYKFTNLLTSILDSAETKN
Mus musculus Kynu transcript variant 1 mRNA (NCBI reference sequence
NM_027552.2; SEQ ID NO: 3):
GAGCAGTTCTTTGGCTAGCTGGGGACAAAGAAAGATCCAGCATCCTCTGAGAAGGTAC
TGAAGACTACTGTCTGGATCTGAGCAGATAACAGTTT(ATGATGGAGCCTTCGCCTCT
TGAGCTTCCAGTTGATGCAGTGCGGCGCATCGCGGCTGAACTCAATTGTGACCCA
ACAGATGAGAGGGTTGCTCTCCGCTTGGATGAGGAAGATAAACTGAGTCATTTTA
GGAACTGTTTTTATATTCCCAAAATGCGGGACCTGCCTTCAATTGATCTATCTTTA
GTGAGTGAGGATGATGATGCCATCTATTTCCTGGGAAATTCCCTTGGCCTTCAAC
CGAAAATGGTTAGGACATACCTGGAGGAAGAACTAGATAAGTGGGCCAAGATGG
GAGCCTATGGCCATGATGTAGGCAAACGCCCTTGGATTGTAGGGGATGAGAGTAT
TGTAAGCCTTATGAAGGACATTGTAGGAGCCCATGAGAAAGAAATAGCTCTAATG
AATGCTTTGACTATTAATTTACATCTCCTGCTGTTATCATTCTTTAAGCCTACTCCA
AAGCGGCACAAAATTCTTCTAGAAGCCAAAGCCTTCCCTTCTGATCATTATGCTAT
TGAGTCACAGATTCAACTTCACGGACTTGATGTTGAGAAAAGTATGCGGATGGTA
AAGCCACGAGAGGGGGAAGAGACCTTAAGGATGGAGGACATACTGGAAGTAATC
GAGGAGGAAGGAGACTCGATCGCCGTGATCCTGTTCAGTGGGCTGCACTTTTATA
CTGGACAGCTGTTCAACATTCCTGCCATAACAAAAGCTGGACATGCAAAGGGCTG
TTTTGTTGGCTTTGACCTAGCACATGCAGTTGGAAATGTTGAACTCCGCTTACATG
ACTGGGGTGTTGACTTTGCCTGCTGGTGTTCCTATAAGTATTTAAATTCAGGAGCT
GGAGGTCTGGCTGGTGCCTTTGTCCACGAGAAACATGCTCATACTGTCAAGCCTG
CGTTAGTGGGATGGTTCGGCCATGACCTCAGTACAAGGTTTAACATGGATAACAA
ACTACAATTAATCCCCGGGGCCAATGGATTCCGAATTTCAAACCCTCCCATTTTGT
TGGTCTGCTCCTTGCACGCCAGTTTAGAGGTCTTTCAGCAAGCAACTATGACTGC
GCTGAGAAGAAAATCCATTCTGCTGACAGGTTATCTGGAATACATGCTCAAACAT
TACCACAGCAAAGATAACACCGAAAACAAGGGGCCGATTGTGAATATCATCACCC
CGTCCAGAGCAGAGGAGCGTGGCTGCCAGTTAACACTCACCTTTTCCATTCCCAA
GAAAAGCGTTTTTAAGGAACTAGAAAAAAGAGGAGTCGTTTGTGACAAGCGAGAA
CCAGATGGCATCCGCGTGGCCCCTGTTCCTCTCTATAATTCTTTCCATGATGTTTA
TAAGTTCATCAGACTGCTCACTTCCATACTCGACTCTTCAGAAAGAAGCTAG)CTAT
ATTTTCTAGCACAACTCAAGTAAATCTCACTGAAAGGTGATGGAGTTTTCACTTCTATT
GAATTTTAGTCATTAAAAAAATCTCCAGAAATTGATTGCACAGAAATGATAACTATAA
AAAAATTTACATAAAACCTGGTGCATGCTTTAATATCTGTGTTTCTGGGGAACGTGGTG
TCCTGTGAATTATGAAGTCACACTTTACATGACTACAGCCTACAGATGACTGTCTTGAT
CAGTTGTCACATTTCATGCTCACTGAAACATTTTCTCTTTAATTTGTGACTGAATTTCCA
ACGTTATAATGTATATGGACTTCTTGTATAAATATTAGAAGTATTACTTTAATTTTGCTA
TAGAGTTTTATTTTAATATTTGTAACTGAATCATCTGAAATATGTTTGATATGATCATGT
TTTATCTAATTCCAGGAGGGGAACAGCCTTTTAAGCTGTTACAAAATCTCTCTCTCTCTC
TCTCTCTCTCTCTCTCTCTCTCTCTCTCTCTCTCTCTCTCTCTCTCTCTTTCCCCCCCCCAG
TGGTGTGTGTGTCTATGTGTTTGTGTTTCTGTGTGTCTGTGTAAAGGACATGTAAGTGCT
TATGTATAAGGGATGAGGTACTTGACCCTATGTACTCTTGTGAGGCCAGAGGTCAACA
CTGGACATCTTCCTCAATCACTGTTTAAAATTTTATTTATTTATTTATTTTTATGTGTATG
GGTATTTTGACTGCATTTATGTCTGTACCTCATGTGCATGCCATGATTACAGAAACTAG
AAGATACATCAGATCACCTGAGACTGGAGTTACAGAGCTGCTGTGTGGATACTAGGAA
TTGAACCCAGGTCGTTTGGAAGAATAGCCAACGCTCTTATTCTTTGACACATCTCTCCA
GCCTTTCCACTTCATATTTCAATACATGATTTCTCCCCAAACCTGGAACTTGCTCCTTCA
GCTCGTGTGGCTGGCCAGTGAGTCTTCAGCGTTTCTCTGTCTCTGCCCTACAATGAATG
CGGGTTACAGCTGTACACTGTTGCACATAGATTTTTTACATGTCTACTGTGATCTGAAC
ACAGTCCTTATATCAGTTCAGCAACCACTTCATCGACCAAGCAATCCCCCAGTCATTGC
TTTTTTGATGCCACTACTAGTATGCATTTACTGGCAAAGAATTCTAAGTTTGTATGTAG
AAAGAAAAAGTTATAATGATTTGATAAACTTGAATAAAACATACTTGGTCAGACAGAA
ACTTCTGATGTGATAAATGATAAGATATGGAACTCTGGCAGTAGCTAACAACAAACAC
AGCACTCTTGTTTACTTAGGAATTCAATTCCGAGTGTTGCACACATATCTATGTTAACA
TAGCAAAGCTTTCCACTGCATTATTTCACCTTCATTAATGAAATGGCTATCAGGACCTG
GAAACTCATCCGTAACACAGATTCCTACATGACTGTTTTTGAGTCCCACAGTGGTCAAC
AAAAGGACATGGTTTTCATTTTCAAGGAACAGAGTACCCTGGTGCCATTCTTCATTGCA
AAAAATATAAAAATAAAATAAATAGTTAATTAT
Mus musculus Kynu isoform 1, 465 amino acids encoded by transcript variant 1 (NCBI
reference sequence NP_081828.1; SEQ ID NO: 4):
MMEPSPLELPVDAVRRIAAELNCDPTDERVALRLDEEDKLSHFRNCFYIPKMRDLPSIDLSL
VSEDDDAIYFLGNSLGLQPKMVRTYLEEELDKWAKMGAYGHDVGKRPWIVGDESIVSLM
KDIVGAHEKEIALMNALTINLHLLLLSFFKPTPKRHKILLEAKAFPSDHYAIESQIQLHGLDV
EKSMRMVKPREGEETLRMEDILEVIEEEGDSIAVILFSGLHFYTGQLFNIPAITKAGHAKGCF
VGFDLAHAVGNVELRLHDWGVDFACWCSYKYLNSGAGGLAGAFVHEKHAHTVKPALVG
WFGHDLSTRFNMDNKLQLIPGANGFRISNPPILLVCSLHASLEVFQQATMTALRRKSILLTG
YLEYMLKHYHSKDNTENKGPIVNIITPSRAEERGCQLTLTFSIPKKSVFKELEKRGVVCDKR
EPDGIRVAPVPLYNSFHDVYKFIRLLTSILDSSERS
Rattus norvegicus Kynu mRNA (NCBI reference sequence NM_053902.2; SEQ ID
NO: 5):
TGAAAAGGTACTGGAAACTGAGGACCCTATCTGGATCAAAGCAGTTTCTG(ATGGAGC
CCTCGCCTCTTGAGCTACCAGTTGATGCAGTGCGGCGCATCGCGGCTGAACTCAA
TTGTGACCCAACCGATGAGAGGGTGGCTCTCCGCTTGGATGAGGAAGATAAACTG
AAGCGTTTTAAGGACTGTTTTTATATCCCCAAAATGCGGGACCTGCCTTCAATTGA
TCTATCTTTAGTGAATGAGGATGATAATGCCATCTATTTCCTGGGAAATTCCCTTG
GTCTTCAACCGAAGATGGTTAAAACATACCTGGAGGAAGAGCTAGATAAGTGGGC
CAAAATAGGAGCCTATGGCCATGAGGTAGGGAAACGTCCTTGGATTATAGGAGAT
GAGAGCATTGTAACCCTTATGAAGGACATTGTAGGAGCCCATGAGAAAGAAATAG
CTCTAATGAATGCTTTGACTGTTAATTTACATCTCCTGCTGTTATCATTCTTTAAGC
CTACACCAAAGCGGCACAAAATTCTTCTAGAAGCCAAAGCCTTCCCTTCTGATCAT
TATGCGATCGAGTCACAGATTCAACTTCATGGACTTGATGTTGAGAAAAGTATGC
GGATGATAAAGCCACGAGAGGGGGAAGAGACCTTAAGAATGGAGGACATACTGG
AAGTAATTGAGAAGGAAGGAGACTCAATTGCTGTGGTCCTGTTCAGTGGCCTGCA
CTTTTATACTGGACAGCTGTTCAACATTCCTGCCATTACACAAGCCGGACATGCAA
AGGGCTGTTTTGTTGGCTTTGACCTAGCGCATGCGGTTGGAAATGTTGAACTCCA
CTTACATGACTGGGATGTTGACTTTGCCTGCTGGTGCTCCTACAAGTATTTAAATT
CAGGAGCTGGAGGTCTGGCTGGTGCCTTCATCCATGAGAAACACGCTCACACGAT
CAAGCCAGCGTTAGTGGGATGGTTCGGCCATGAACTCAGTACAAGATTTAACATG
GATAACAAACTACAATTAATCCCCGGGGTCAATGGATTCCGAATTTCCAACCCTCC
CATTCTGTTGGTCTGCTCCTTGCATGCCAGTTTAGAGATCTTTCAGCAAGCAACTA
TGACTGCGCTGAGGAGAAAATCCATTCTGCTGACAGGTTATCTGGAATACTTGCT
CAAACATTACCATGGCGGAAATGACACAGAAAACAAGAGGCCAGTTGTGAACATA
ATCACCCCATCCAGAGCAGAGGAACGAGGCTGCCAGCTGACACTGACCTTTTCCA
TTTCCAAGAAAGGCGTTTTTAAGGAACTAGAAAAAAGAGGAGTCGTCTGTGACAA
GCGAGAACCAGAAGGCATCCGGGTGGCCCCGGTTCCTCTCTATAATTCTTTCCAT
GATGTTTATAAGTTCATCAGACTGCTTACTGCCATACTCGACTCTACAGAAAGAAA
CTAG)CCATGCTTTCTAAATAACTCAAGTAAATCTCACACACTGGGGGTTCCACTTCTA
CTGCATTTTAGTCATTCAAAAGTCTCCAGAAATTGATGGCATAGAAATGATGATGATTT
TATAAACTTACATAAAACCTGGTACATGTTTTAATATCTGTGTCGCTGATGTGCTGTGG
ACTAAGAAGTCACATTTTACATGACTCCAACCTACAGATGACTGTCTTGATCAGCTGTC
ACCTTCCATGGTCACTGAAAGGTTGTGTGTTTAATTTGTGACTGAAATGACAACATTAA
AATGTATCTGGACTTCTTGTATAAAAAAA
Rattus norvegicus Kynu amino acid, 464 amino acids (NCBI reference sequence
NP_446354.1; SEQ ID NO: 6):
MEPSPLELPVDAVRRIAAELNCDPTDERVALRLDEEDKLKRFKDCFYIPKMRDLPSIDLSLV
NEDDNAIYFLGNSLGLQPKMVKTYLEEELDKWAKIGAYGHEVGKRPWIIGDESIVTLMKDI
VGAHEKEIALMNALTVNLHLLLLSFFKPTPKRHKILLEAKAFPSDHYAIESQIQLHGLDVEK
SMRMIKPREGEETLRMEDILEVIEKEGDSIAVVLFSGLHFYTGQLFNIPAITQAGHAKGCFV
GFDLAHAVGNVELHLHDWDVDFACWCSYKYLNSGAGGLAGAFIHEKHAHTIKPALVGW
FGHELSTRFNMDNKLQLIPGVNGFRISNPPILLVCSLHASLEIFQQATMTALRRKSILLTGYL
EYLLKHYHGGNDTENKRPVVNIITPSRAEERGCQLTLTFSISKKGVFKELEKRGVVCDKREP
EGIRVAPVPLYNSFHDVYKFIRLLTAILDSTERN
Exemplary mutant Mus musculus Kynu mRNA (SEQ ID NO: 7)
GAGCAGTTCTTTGGCTAGCTGGGGACAAAGAAAGATCCAGCATCCTCTGAGAAGGTAC
TGAAGACTACTGTCTGGATCTGAGCAGATAACAGTTT(ATGATGGAGCCTTCGCCTCT
TGAGCTTCCAGTTGATGCAGTGCGGCGCATCGCGGCTGAACTCAATTGTGACCCA
ACAGATGAGAGGGTTGCTCTCCGCTTGGATGAGGAAGATAAACTGAGTCATTTTA
GGAACTGTTTTTATATTCCCAAAATGCGGGACCTGCCTTCAATTGATCTATCTTTA
GTGAGTGAGGATGATGATGCCATCTATTTCCTGGGAAATTCCCTTGGCCTTCAAC
CGAAAATGGTTAGGACATACCTGGAGGAAGAGCTTGAAAAATGGGCTAAGATGG
GAGCCTATGGCCATGATGTAGGCAAACGCCCTTGGATTGTAGGGGATGAGAGTAT
TGTAAGCCTTATGAAGGACATTGTAGGAGCCCATGAGAAAGAAATAGCTCTAATG
AATGCTTTGACTATTAATTTACATCTCCTGCTGTTATCATTCTTTAAGCCTACTCCA
AAGCGGCACAAAATTCTTCTAGAAGCCAAAGCCTTCCCTTCTGATCATTATGCTAT
TGAGTCACAGATTCAACTTCACGGACTTGATGTTGAGAAAAGTATGCGGATGGTA
AAGCCACGAGAGGGGGAAGAGACCTTAAGGATGGAGGACATACTGGAAGTAATC
GAGGAGGAAGGAGACTCGATCGCCGTGATCCTGTTCAGTGGGCTGCACTTTTATA
CTGGACAGCTGTTCAACATTCCTGCCATAACAAAAGCTGGACATGCAAAGGGCTG
TTTTGTTGGCTTTGACCTAGCACATGCAGTTGGAAATGTTGAACTCCGCTTACATG
ACTGGGGTGTTGACTTTGCCTGCTGGTGTTCCTATAAGTATTTAAATTCAGGAGCT
GGAGGTCTGGCTGGTGCCTTTGTCCACGAGAAACATGCTCATACTGTCAAGCCTG
CGTTAGTGGGATGGTTCGGCCATGACCTCAGTACAAGGTTTAACATGGATAACAA
ACTACAATTAATCCCCGGGGCCAATGGATTCCGAATTTCAAACCCTCCCATTTTGT
TGGTCTGCTCCTTGCACGCCAGTTTAGAGGTCTTTCAGCAAGCAACTATGACTGC
GCTGAGAAGAAAATCCATTCTGCTGACAGGTTATCTGGAATACATGCTCAAACAT
TACCACAGCAAAGATAACACCGAAAACAAGGGGCCGATTGTGAATATCATCACCC
CGTCCAGAGCAGAGGAGCGTGGCTGCCAGTTAACACTCACCTTTTCCATTCCCAA
GAAAAGCGTTTTTAAGGAACTAGAAAAAAGAGGAGTCGTTTGTGACAAGCGAGAA
CCAGATGGCATCCGCGTGGCCCCTGTTCCTCTCTATAATTCTTTCCATGATGTTTA
TAAGTTCATCAGACTGCTCACTTCCATACTCGACTCTTCAGAAAGAAGCTAG)CTAT
ATTTTCTAGCACAACTCAAGTAAATCTCACTGAAAGGTGATGGAGTTTTCACTTCTATT
GAATTTTAGTCATTAAAAAAATCTCCAGAAATTGATTGCACAGAAATGATAACTATAA
AAAAATTTACATAAAACCTGGTGCATGCTTTAATATCTGTGTTTCTGGGGAACGTGGTG
TCCTGTGAATTATGAAGTCACACTTTACATGACTACAGCCTACAGATGACTGTCTTGAT
CAGTTGTCACATTTCATGCTCACTGAAACATTTTCTCTTTAATTTGTGACTGAATTTCCA
ACGTTATAATGTATATGGACTTCTTGTATAAATATTAGAAGTATTACTTTAATTTTGCTA
TAGAGTTTTATTTTAATATTTGTAACTGAATCATCTGAAATATGTTTGATATGATCATGT
TTTATCTAATTCCAGGAGGGGAACAGCCTTTTAAGCTGTTACAAAATCTCTCTCTCTCTC
TCTCTCTCTCTCTCTCTCTCTCTCTCTCTCTCTCTCTCTCTCTCTCTCTTTCCCCCCCCCAG
TGGTGTGTGTGTCTATGTGTTTGTGTTTCTGTGTGTCTGTGTAAAGGACATGTAAGTGCT
TATGTATAAGGGATGAGGTACTTGACCCTATGTACTCTTGTGAGGCCAGAGGTCAACA
CTGGACATCTTCCTCAATCACTGTTTAAAATTTTATTTATTTATTTATTTTTATGTGTATG
GGTATTTTGACTGCATTTATGTCTGTACCTCATGTGCATGCCATGATTACAGAAACTAG
AAGATACATCAGATCACCTGAGACTGGAGTTACAGAGCTGCTGTGTGGATACTAGGAA
TTGAACCCAGGTCGTTTGGAAGAATAGCCAACGCTCTTATTCTTTGACACATCTCTCCA
GCCTTTCCACTTCATATTTCAATACATGATTTCTCCCCAAACCTGGAACTTGCTCCTTCA
GCTCGTGTGGCTGGCCAGTGAGTCTTCAGCGTTTCTCTGTCTCTGCCCTACAATGAATG
CGGGTTACAGCTGTACACTGTTGCACATAGATTTTTTACATGTCTACTGTGATCTGAAC
ACAGTCCTTATATCAGTTCAGCAACCACTTCATCGACCAAGCAATCCCCCAGTCATTGC
TTTTTTGATGCCACTACTAGTATGCATTTACTGGCAAAGAATTCTAAGTTTGTATGTAG
AAAGAAAAAGTTATAATGATTTGATAAACTTGAATAAAACATACTTGGTCAGACAGAA
ACTTCTGATGTGATAAATGATAAGATATGGAACTCTGGCAGTAGCTAACAACAAACAC
AGCACTCTTGTTTACTTAGGAATTCAATTCCGAGTGTTGCACACATATCTATGTTAACA
TAGCAAAGCTTTCCACTGCATTATTTCACCTTCATTAATGAAATGGCTATCAGGACCTG
GAAACTCATCCGTAACACAGATTCCTACATGACTGTTTTTGAGTCCCACAGTGGTCAAC
AAAAGGACATGGTTTTCATTTTCAAGGAACAGAGTACCCTGGTGCCATTCTTCATTGCA
AAAAATATAAAAATAAAATAAATAGTTAATTAT
Exemplary mutant Mus musculus Kynu polypeptide, 465 amino acids encoded by
mutant Mus musculus Kynu mRNA (SEQ ID NO: 8):
MMEPSPLELPVDAVRRIAAELNCDPTDERVALRLDEEDKLSHFRNCFYIPKMRDLPSIDLSL
VSEDDDAIYFLGNSLGLQPKMVRTYLEEELEKWAKMGAYGHDVGKRPWIVGDESIVSLM
KDIVGAHEKEIALMNALTINLHLLLLSFFKPTPKRHKILLEAKAFPSDHYAIESQIQLHGLDV
EKSMRMVKPREGEETLRMEDILEVIEEEGDSIAVILFSGLEIFYTGQLFNIPAITKAGHAKGCF
VGFDLAHAVGNVELRLHDWGVDFACWCSYKYLNSGAGGLAGAFVHEKHAHTVKPALVG
WFGHDLSTRFNMDNKLQLIPGANGFRISNPPILLVCSLHASLEVFQQATMTALRRKSILLTG
YLEYMLKHYHSKDNTENKGPIVNIITPSRAEERGCQLTLTFSIPKKSVFKELEKRGVVCDKR
EPDGIRVAPVPLYNSFHDVYKFIRLLTSILDSSERS
Exemplary portion of a disrupted Mus musculus Kynu allele including a self-deleting
neomycin selection cassette (mouse sequence indicated in uppercase font and targeting vector
sequence indicated in lowercase font; SEQ ID NO: 9):
TAATGGTGGACTCTGTAGAAGGCTGATATTCTGCAGAAAAAAAAATGATGATGGCTAC
ATTATTTCAACGTTTTACTTCCTTCTTAGATAACAGTTTATGggtaccgatttaaatgatccagtggtcctg
cagaggagagattgggagaatcccggtgtgacacagctgaacagactagccgcccaccctccctttgcttcttggagaaacagtgaggaagct
aggacagacagaccaagccagcaactcagatctttgaacggggagtggagatttgcctggtttccggcaccagaagcggtgccggaaagctg
gctggagtgcgatcttcctgaggccgatactgtcgtcgtcccctcaaactggcagatgcacggttacgatgcgcccatctacaccaacgtgacct
atcccattacggtcaatccgccgtttgttcccacggagaatccgacgggttgttactcgctcacatttaatgttgatgaaagctggctacaggaagg
ccagacgcgaattatttttgatggcgttaactcggcgtttcatctgtggtgcaacgggcgctgggtcggttacggccaggacagtcgtttgccgtct
gaatttgacctgagcgcatttttacgcgccggagaaaaccgcctcgcggtgatggtgctgcgctggagtgacggcagttatctggaagatcagg
atatgtggcggatgagcggcattttccgtgacgtctcgttgctgcataaaccgactacacaaatcagcgatttccatgttgccactcgctttaatgat
gatttcagccgcgctgtactggaggctgaagttcagatgtgcggcgagttgcgtgactacctacgggtaacagtttctttatggcagggtgaaac
gcaggtcgccagcggcaccgcgcctttcggcggtgaaattatcgatgagcgtggtggttatgccgatcgcgtcacactacgtctgaacgtcgaa
aacccgaaactgtggagcgccgaaatcccgaatctctatcgtgcggtggttgaactgcacaccgccgacggcacgctgattgaagcagaagc
ctgcgatgtcggtttccgcgaggtgcggattgaaaatggtctgctgctgctgaacggcaagccgttgctgattcgaggcgttaaccgtcacgagc
atcatcctctgcatggtcaggtcatggatgagcagacgatggtgcaggatatcctgctgatgaagcagaacaactttaacgccgtgcgctgttcg
cattatccgaaccatccgctgtggtacacgctgtgcgaccgctacggcctgtatgtggtggatgaagccaatattgaaacccacggcatggtgcc
aatgaatcgtctgaccgatgatccgcgctggctaccggcgatgagcgaacgcgtaacgcgaatggtgcagcgcgatcgtaatcacccgagtgt
gatcatctggtcgctggggaatgaatcaggccacggcgctaatcacgacgcgctgtatcgctggatcaaatctgtcgatccttcccgcccggtgc
agtatgaaggcggcggagccgacaccacggccaccgatattatttgcccgatgtacgcgcgcgtggatgaagaccagcccttcccggctgtg
ccgaaatggtccatcaaaaaatggctttcgctacctggagagacgcgcccgctgatcctttgcgaatacgcccacgcgatgggtaacagtcttg
gcggtttcgctaaatactggcaggcgtttcgtcagtatccccgtttacagggcggcttcgtctgggactgggtggatcagtcgctgattaaatatga
tgaaaacggcaacccgtggtcggcttacggcggtgattttggcgatacgccgaacgatcgccagttctgtatgaacggtctggtctttgccgacc
gcacgccgcatccagcgctgacggaagcaaaacaccagcagcagtttttccagttccgtttatccgggcaaaccatcgaagtgaccagcgaat
acctgttccgtcatagcgataacgagctcctgcactggatggtggcgctggatggtaagccgctggcaagcggtgaagtgcctctggatgtcgc
tccacaaggtaaacagttgattgaactgcctgaactaccgcagccggagagcgccgggcaactctggctcacagtacgcgtagtgcaaccgaa
cgcgaccgcatggtcagaagccgggcacatcagcgcctggcagcagtggcgtctggcggaaaacctcagtgtgacgctccccgccgcgtcc
cacgccatcccgcatctgaccaccagcgaaatggatttttgcatcgagctgggtaataagcgttggcaatttaaccgccagtcaggctttctttcac
agatgtggattggcgataaaaaacaactgctgacgccgctgcgcgatcagttcacccgtgcaccgctggataacgacattggcgtaagtgaag
cgacccgcattgaccctaacgcctgggtcgaacgctggaaggcggcgggccattaccaggccgaagcagcgttgttgcagtgcacggcaga
tacacttgctgatgcggtgctgattacgaccgctcacgcgtggcagcatcaggggaaaaccttatttatcagccggaaaacctaccggattgatg
gtagtggtcaaatggcgattaccgttgatgttgaagtggcgagcgatacaccgcatccggcgcggattggcctgaactgccagctggcgcagg
tagcagagcgggtaaactggctcggattagggccgcaagaaaactatcccgaccgccttactgccgcctgttttgaccgctgggatctgccattg
tcagacatgtataccccgtacgtcttcccgagcgaaaacggtctgcgctgcgggacgcgcgaattgaattatggcccacaccagtggcgcggc
gacttccagttcaacatcagccgctacagtcaacagcaactgatggaaaccagccatcgccatctgctgcacgcggaagaaggcacatggctg
aatatcgacggtttccatatggggattggtggcgacgactcctggagcccgtcagtatcggcggaattccagctgagcgccggtcgctaccatta
ccagttggtctggtgtcaaaaataataataaccgggcaggggggatctaagctctagataagtaatgatcataatcagccatatcacatctgtaga
ggttttacttgctttaaaaaacctcccacacctccccctgaacctgaaacataaaatgaatgcaattgttgttgttaacttgtttattgcagcttataa
tggttacaaataaagcaatagcatcacaaatttcacaaataaagcatttttttcactgcattctagttgtggtttgtccaaactcatcaatgtatctta
tcatgtctggatcccccggctagagtttaaacactagaactagtggatccccgggctcgataactataacggtcctaaggtagcgactcgacataactt
cgtataatgtatgctatacgaagttatatgcatgccagtagcagcacccacgtccaccttctgtctagtaatgtccaacacctccctcagtccaaacac
tgctctgcatccatgtggctcccatttatacctgaagcacttgatggggcctcaatgttttactagagcccacccccctgcaactctgagaccctctg
gatttgtctgtcagtgcctcactggggcgttggataatttcttaaaaggtcaagttccctcagcagcattctctgagcagtctgaagatgtgtgctttt
cacagttcaaatccatgtggctgtttcacccacctgcctggccttgggttatctatcaggacctagcctagaagcaggtgtgtggcacttaacaccta
agctgagtgactaactgaacactcaagtggatgccatctttgtcacttcttgactgtgacacaagcaactcctgatgccaaagccctgcccacccc
tctcatgcccatatttggacatggtacaggtcctcactggccatggtctgtgaggtcctggtcctctttgacttcataattcctaggggccactagtat
ctataagaggaagagggtgctggctcccaggccacagcccacaaaattccacctgctcacaggttggctggctcgacccaggtggtgtcccct
gctctgagccagctcccggccaagccagcaccatgggaacccccaagaagaagaggaaggtgcgtaccgatttaaattccaatttactgaccg
tacaccaaaatttgcctgcattaccggtcgatgcaacgagtgatgaggttcgcaagaacctgatggacatgttcagggatcgccaggcgttttctg
agcatacctggaaaatgcttctgtccgtttgccggtcgtgggcggcatggtgcaagttgaataaccggaaatggtttcccgcagaacctgaagat
gttcgcgattatcttctatatcttcaggcgcgcggtctggcagtaaaaactatccagcaacatttgggccagctaaacatgcttcatcgtcggtccg
ggctgccacgaccaagtgacagcaatgctgtttcactggttatgcggcggatccgaaaagaaaacgttgatgccggtgaacgtgcaaaacagg
ctctagcgttcgaacgcactgatttcgaccaggttcgttcactcatggaaaatagcgatcgctgccaggatatacgtaatctggcatttctggggatt
gcttataacaccctgttacgtatagccgaaattgccaggatcagggttaaagatatctcacgtactgacggtgggagaatgttaatccatattggca
gaacgaaaacgctggttagcaccgcaggtgtagagaaggcacttagcctgggggtaactaaactggtcgagcgatggatttccgtctctggtgt
agctgatgatccgaataactacctgttttgccgggtcagaaaaaatggtgttgccgcgccatctgccaccagccagctatcaactcgcgccctgg
aagggatttttgaagcaactcatcgattgatttacggcgctaaggtaaatataaaatttttaagtgtataatgtgttaaactactgattctaattgttt
gtgtattttaggatgactctggtcagagatacctggcctggtctggacacagtgcccgtgtcggagccgcgcgagatatggcccgcgctggagtttca
ataccggagatcatgcaagctggtggctggaccaatgtaaatattgtcatgaactatatccgtaacctggatagtgaaacaggggcaatggtgcg
cctgctggaagatggcgattgatctagataagtaatgatcataatcagccatatcacatctgtagaggttttacttgctttaaaaaacctcccacacct
ccccctgaacctgaaacataaaatgaatgcaattgttgttgttaaacctgccctagttgcggccaattccagctgagcgtgagctcaccattaccag
ttggtctggtgtcaaaaataataataaccgggcaggggggatctaagctctagataagtaatgatcataatcagccatatcacatctgtagaggtttt
acttgctttaaaaaacctcccacacctccccctgaacctgaaacataaaatgaatgcaattgttgttgttaacttgtttattgcagcttataatggtta
caaataaagcaatagcatcacaaatttcacaaataaagcatttttttcactgcattctagttgtggtttgtccaaactcatcaatgtatcttatcatgt
ctggatcccccggctagagtttaaacactagaactagtggatcccccgggatcatggcctccgcgccgggttttggcgcctcccgcgggcgcccccctc
ctcacggcgagcgctgccacgtcagacgaagggcgcagcgagcgtcctgatccttccgcccggacgctcaggacagcggcccgctgctcat
aagactcggccttagaaccccagtatcagcagaaggacattttaggacgggacttgggtgactctagggcactggttttctttccagagagcgga
acaggcgaggaaaagtagtcccttctcggcgattctgcggagggatctccgtggggcggtgaacgccgatgattatataaggacgcgccgggt
gtggcacagctagttccgtcgcagccgggatttgggtcgcggttcttgtttgtggatcgctgtgatcgtcacttggtgagtagcgggctgctgggc
tggccggggctttcgtggccgccgggccgctcggtgggacggaagcgtgtggagagaccgccaagggctgtagtctgggtccgcgagcaa
ggttgccctgaactgggggttggggggagcgcagcaaaatggcggctgttcccgagtcttgaatggaagacgcttgtgaggcgggctgtgag
gtcgttgaaacaaggtggggggcatggtgggcggcaagaacccaaggtcttgaggccttcgctaatgcgggaaagctcttattcgggtgagat
gggctggggcaccatctggggaccctgacgtgaagtttgtcactgactggagaactcggtttgtcgtctgttgcgggggcggcagttatggcgg
tgccgttgggcagtgcacccgtacctttgggagcgcgcgccctcgtcgtgtcgtgacgtcacccgttctgttggcttataatgcagggtggggcc
acctgccggtaggtgtgcggtaggcttttctccgtcgcaggacgcagggttcgggcctagggtaggctctcctgaatcgacaggcgccggacc
tctggtgaggggagggataagtgaggcgtcagtttctttggtcggttttatgtacctatcttcttaagtagctgaagctccggttrtgaactatgcgct
cggggttggcgagtgtgttttgtgaagttttttaggcaccttttgaaatgtaatcatttgggtcaatatgtaattttcagtgttagactagtaaattgt
ccgctaaattctggccgtttttggcttattgttagacgtgttgacaattaatcatcggcatagtatatcggcatagtataatacgacaaggtgaggaac
taaaccatgggatcggccattgaacaagatggattgcacgcaggttctccggccgcttgggtggagaggctattcggctatgactgggcacaacag
acaatcggctgctctgatgccgccgtgttccggctgtcagcgcaggggcgcccggttctttttgtcaagaccgacctgtccggtgccctgaatga
actgcaggacgaggcagcgcggctatcgtggctggccacgacgggcgttccttgcgcagctgtgctcgacgttgtcactgaagcgggaaggg
actggctgctattgggcgaagtgccggggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagcacgtactcggatggaagccggtctt
gtcgatcaggatgatctggacgaagagcatcaggggctcgcgccagccgaactgttcgccaggctcaaggcgcgcatgcccgacggcgatg
atctcgtcgtgacccatggcgatgcctgcttgccgaatatcatggtggaaaatggccgcttttctggattcatcgactgtggccggctgggtgtgg
cggaccgctatcaggacatagcgttggctacccgtgatattgctgaagagcttggcggcgaatgggctgaccgcttcctcgtgctttacggtatc
gccgctcccgattcgcagcgcatcgccttctatcgccttcttgacgagttcttctgaggggatccgctgtaagtctgcagaaattgatgatctattaa
acaataaagatgtccactaaaatggaagtttttcctgtcatactttgttaagaagggtgagaacagagtacctacattttgaatggaaggattggagc
tacgggggtgggggtggggtgggattagataaatgcctgctattactgaaggctattactattgattatgataatgtttcatagttggatatcataa
tttaaacaagcaaaaccaaattaagggccagctcattcctcccactcatgatctatagatctatagatctctcgtgggatcattgtttttctcttgatt
cccactttgtggttctaagtactgtggtttccaaatgtgtcagtttcatagcctgaagaacgagatcagcagcctctgttccacatacacttcattctc
agtattgttttgccaagttctaattccatcagacctcgacctgcagcccctagataacttcgtataatgtatgctatacgaagttatGCTAGCGAG
AGGTATCTGTGAAAGAAAGAAATGCTCATTAGACTTCCATTTTGTGTTCACTTATGTCC
CTCAAAAGTATATTATCTTCATGGCTCTGATGTAACAA
Exemplary portion of a disrupted Mus musculus Kynu allele including a self-deleting
hygromycin selection cassette (mouse sequence indicated in uppercase font and targeting vector
sequence indicated in lowercase font; SEQ ID NO: 10):
TAATGGTGGACTCTGTAGAAGGCTGATATTCTGCAGAAAAAAAAATGATGATGGCTAC
ATTATTTCAACGTTTTACTTCCTTCTTAGATAACAGTTTATGggtaccgatttaaatgatccagtggtcctg
cagaggagagattgggagaatcccggtgtgacacagctgaacagactagccgcccaccctccctttgcttcttggagaaacagtgaggaagct
aggacagacagaccaagccagcaactcagatctttgaacggggagtggagatttgcctggtttccggcaccagaagcggtgccggaaagctg
gctggagtgcgatcttcctgaggccgatactgtcgtcgtcccctcaaactggcagatgcacggttacgatgcgcccatctacaccaacgtgacct
atcccattacggtcaatccgccgtttgttcccacggagaatccgacgggttgttactcgctcacatttaatgttgatgaaagctggctacaggaagg
ccagacgcgaattatttttgatggcgttaactcggcgtttcatctgtggtgcaacgggcgctgggtcggttacggccaggacagtcgtttgccgtct
gaatttgacctgagcgcatttttacgcgccggagaaaaccgcctcgcggtgatggtgctgcgctggagtgacggcagttatctggaagatcagg
atatgtggcggatgagcggcattttccgtgacgtctcgttgctgcataaaccgactacacaaatcagcgatttccatgttgccactcgctttaatgat
gatttcagccgcgctgtactggaggctgaagttcagatgtgcggcgagttgcgtgactacctacgggtaacagtttctttatggcagggtgaaac
gcaggtcgccagcggcaccgcgcctttcggcggtgaaattatcgatgagcgtggtggttatgccgatcgcgtcacactacgtctgaacgtcgaa
aacccgaaactgtggagcgccgaaatcccgaatctctatcgtgcggtggttgaactgcacaccgccgacggcacgctgattgaagcagaagc
ctgcgatgtcggtttccgcgaggtgcggattgaaaatggtctgctgctgctgaacggcaagccgttgctgattcgaggcgttaaccgtcacgagc
atcatcctctgcatggtcaggtcatggatgagcagacgatggtgcaggatatcctgctgatgaagcagaacaactttaacgccgtgcgctgttcg
cattatccgaaccatccgctgtggtacacgctgtgcgaccgctacggcctgtatgtggtggatgaagccaatattgaaacccacggcatggtgcc
aatgaatcgtctgaccgatgatccgcgctggctaccggcgatgagcgaacgcgtaacgcgaatggtgcagcgcgatcgtaatcacccgagtgt
gatcatctggtcgctggggaatgaatcaggccacggcgctaatcacgacgcgctgtatcgctggatcaaatctgtcgatccttcccgcccggtgc
agtatgaaggcggcggagccgacaccacggccaccgatattatttgcccgatgtacgcgcgcgtggatgaagaccagcccttcccggctgtg
ccgaaatggtccatcaaaaaatggctttcgctacctggagagacgcgcccgctgatcctttgcgaatacgcccacgcgatgggtaacagtcttg
gcggtttcgctaaatactggcaggcgtttcgtcagtatccccgtttacagggcggcttcgtctgggactgggtggatcagtcgctgattaaatatga
tgaaaacggcaacccgtggtcggcttacggcggtgattttggcgatacgccgaacgatcgccagttctgtatgaacggtctggtctttgccgacc
gcacgccgcatccagcgctgacggaagcaaaacaccagcagcagtttttccagttccgtttatccgggcaaaccatcgaagtgaccagcgaat
acctgttccgtcatagcgataacgagctcctgcactggatggtggcgctggatggtaagccgctggcaagcggtgaagtgcctctggatgtcgc
tccacaaggtaaacagttgattgaactgcctgaactaccgcagccggagagcgccgggcaactctggctcacagtacgcgtagtgcaaccgaa
cgcgaccgcatggtcagaagccgggcacatcagcgcctggcagcagtggcgtctggcggaaaacctcagtgtgacgctccccgccgcgtcc
cacgccatcccgcatctgaccaccagcgaaatggatttttgcatcgagctgggtaataagcgttggcaatttaaccgccagtcaggctttctttcac
agatgtggattggcgataaaaaacaactgctgacgccgctgcgcgatcagttcacccgtgcaccgctggataacgacattggcgtaagtgaag
cgacccgcattgaccctaacgcctgggtcgaacgctggaaggcggcgggccattaccaggccgaagcagcgttgttgcagtgcacggcaga
tacacttgctgatgcggtgctgattacgaccgctcacgcgtggcagcatcaggggaaaaccttatttatcagccggaaaacctaccggattgatg
gtagtggtcaaatggcgattaccgttgatgttgaagtggcgagcgatacaccgcatccggcgcggattggcctgaactgccagctggcgcagg
tagcagagcgggtaaactggctcggattagggccgcaagaaaactatcccgaccgccttactgccgcctgttttgaccgctgggatctgccattg
tcagacatgtataccccgtacgtcttcccgagcgaaaacggtctgcgctgcgggacgcgcgaattgaattatggcccacaccagtggcgcggc
gacttccagttcaacatcagccgctacagtcaacagcaactgatggaaaccagccatcgccatctgctgcacgcggaagaaggcacatggctg
aatatcgacggtttccatatggggattggtggcgacgactcctggagcccgtcagtatcggcggaattccagctgagcgccggtcgctaccatta
ccagttggtctggtgtcaaaaataataataaccgggcaggggggatctaagctctagataagtaatgatcataatcagccatatcacatctgtaga
ggttttacttgctttaaaaaacctcccacacctccccctgaacctgaaacataaaatgaatgcaattgttgttgttaacttgtttattgcagcttataa
tggttacaaataaagcaatagcatcacaaatttcacaaataaagcatttttttcactgcattctagttgtggtttgtccaaactcatcaatgtatctta
tcatgtctggatcccccggctagagtttaaacactagaactagtggatccccgggctcgataactataacggtcctaaggtagcgactcgacataactt
cgtataatgtatgctatacgaagttatatgcatgccagtagcagcacccacgtccaccttctgtctagtaatgtccaacacctccctcagtccaaacac
tgctctgcatccatgtggctcccatttatacctgaagcacttgatggggcctcaatgttttactagagcccacccccctgcaactctgagaccctctg
gatttgtctgtcagtgcctcactggggcgttggataatttcttaaaaggtcaagttccctcagcagcattctctgagcagtctgaagatgtgtgcttt
tcacagttcaaatccatgtggctgtttcacccacctgcctggccttgggttatctatcaggacctagcctagaagcaggtgtgtggcacttaacaccta
agctgagtgactaactgaacactcaagtggatgccatctttgtcacttcttgactgtgacacaagcaactcctgatgccaaagccctgcccacccc
tctcatgcccatatttggacatggtacaggtcctcactggccatggtctgtgaggtcctggtcctctttgacttcataattcctaggggccactagtat
ctataagaggaagagggtgctggctcccaggccacagcccacaaaattccacctgctcacaggttggctggctcgacccaggtggtgtcccct
gctctgagccagctcccggccaagccagcaccatgggaacccccaagaagaagaggaaggtgcgtaccgatttaaattccaatttactgaccg
tacaccaaaatttgcctgcattaccggtcgatgcaacgagtgatgaggttcgcaagaacctgatggacatgttcagggatcgccaggcgttttctg
agcatacctggaaaatgcttctgtccgtttgccggtcgtgggcggcatggtgcaagttgaataaccggaaatggtttcccgcagaacctgaagat
gttcgcgattatcttctatatcttcaggcgcgcggtctggcagtaaaaactatccagcaacatttgggccagctaaacatgcttcatcgtcggtccg
ggctgccacgaccaagtgacagcaatgctgtttcactggttatgcggcggatccgaaaagaaaacgttgatgccggtgaacgtgcaaaacagg
ctctagcgttcgaacgcactgatttcgaccaggttcgttcactcatggaaaatagcgatcgctgccaggatatacgtaatctggcatttctggggatt
gcttataacaccctgttacgtatagccgaaattgccaggatcagggttaaagatatctcacgtactgacggtgggagaatgttaatccatattggca
gaacgaaaacgctggttagcaccgcaggtgtagagaaggcacttagcctgggggtaactaaactggtcgagcgatggatttccgtctctggtgt
agctgatgatccgaataactacctgttttgccgggtcagaaaaaatggtgttgccgcgccatctgccaccagccagctatcaactcgcgccctgg
aagggatttttgaagcaactcatcgattgatttacggcgctaaggtaaatataaaatttttaagtgtataatgtgttaaactactgattctaattgttt
gtgtattttaggatgactctggtcagagatacctggcctggtctggacacagtgcccgtgtcggagccgcgcgagatatggcccgcgctggagtttca
ataccggagatcatgcaagctggtggctggaccaatgtaaatattgtcatgaactatatccgtaacctggatagtgaaacaggggcaatggtgcg
cctgctggaagatggcgattgatctagataagtaatgatcataatcagccatatcacatctgtagaggttttacttgctttaaaaaacctcccacacct
ccccctgaacctgaaacataaaatgaatgcaattgttgttgttaaacctgccctagttgcggccaattccagctgagcgtgagctcaccattaccag
ttggtctggtgtcaaaaataataataaccgggcaggggggatctaagctctagataagtaatgatcataatcagccatatcacatctgtagaggtttt
acttgctttaaaaaacctcccacacctccccctgaacctgaaacataaaatgaatgcaattgttgttgttaacttgtttattgcagcttataatggtta
caaataaagcaatagcatcacaaatttcacaaataaagcatttttttcactgcattctagttgtggtttgtccaaactcatcaatgtatcttatcatgt
ctggatcccccggctagagtttaaacactagaactagtggatcccccgggatcatggcctccgcgccgggttttggcgcctcccgcgggcgcccccctc
ctcacggcgagcgctgccacgtcagacgaagggcgcagcgagcgtcctgatccttccgcccggacgctcaggacagcggcccgctgctcat
aagactcggccttagaaccccagtatcagcagaaggacattttaggacgggacttgggtgactctagggcactggttttctttccagagagcgga
acaggcgaggaaaagtagtcccttctcggcgattctgcggagggatctccgtggggcggtgaacgccgatgattatataaggacgcgccgggt
gtggcacagctagttccgtcgcagccgggatttgggtcgcggttcttgtttgtggatcgctgtgatcgtcacttggtgagtagcgggctgctgggc
tggccggggctttcgtggccgccgggccgctcggtgggacggaagcgtgtggagagaccgccaagggctgtagtctgggtccgcgagcaa
ggttgccctgaactgggggttggggggagcgcagcaaaatggcggctgttcccgagtcttgaatggaagacgcttgtgaggcgggctgtgag
gtcgttgaaacaaggtggggggcatggtgggcggcaagaacccaaggtcttgaggccttcgctaatgcgggaaagctcttattcgggtgagat
gggctggggcaccatctggggaccctgacgtgaagtttgtcactgactggagaactcggtttgtcgtctgttgcgggggcggcagttatggcgg
tgccgttgggcagtgcacccgtacctttgggagcgcgcgccctcgtcgtgtcgtgacgtcacccgttctgttggcttataatgcagggtggggcc
acctgccggtaggtgtgcggtaggcttttctccgtcgcaggacgcagggttcgggcctagggtaggctctcctgaatcgacaggcgccggacc
tctggtgaggggagggataagtgaggcgtcagtttctttggtcggttttatgtacctatcttcttaagtagctgaagctccggttttgaactatgcgct
cggggttggcgagtgtgttttgtgaagttttttaggcaccttttgaaatgtaatcatttgggtcaatatgtaattttcagtgttagactagtaaattgt
ccgctaaattctggccgtttttggcttttttgttagacgtgttgacaattaatcatcggcatagtatatcggcatagtataatacgacaaggtgaggaa
ctaaaccatgaaaaagcctgaactcaccgcgacgtctgtcgagaagtttctgatcgaaaagttcgacagcgtgtccgacctgatgcagctctcggagg
gcgaagaatctcgtgctttcagcttcgatgtaggagggcgtggatatgtcctgcgggtaaatagctgcgccgatggtttctacaaagatcgttatgt
ttatcggcactttgcatcggccgcgctcccgattccggaagtgcttgacattggggaattcagcgagagcctgacctattgcatctcccgccgtgc
acagggtgtcacgttgcaagacctgcctgaaaccgaactgcccgctgttctgcagccggtcgcggaggccatggatgcgatcgctgcggccg
atcttagccagacgagcgggttcggcccattcggaccgcaaggaatcggtcaatacactacatggcgtgatttcatatgcgcgattgctgatccc
catgtgtatcactggcaaactgtgatggacgacaccgtcagtgcgtccgtcgcgcaggctctcgatgagctgatgctttgggccgaggactgcc
ccgaagtccggcacctcgtgcacgcggatttcggctccaacaatgtcctgacggacaatggccgcataacagcggtcattgactggagcgagg
cgatgttcggggattcccaatacgaggtcgccaacatcttcttctggaggccgtggttggcttgtatggagcagcagacgcgctacttcgagcgg
aggcatccggagcttgcaggatcgccgcggctccgggcgtatatgctccgcattggtcttgaccaactctatcagagcttggttgacggcaatttc
gatgatgcagcttgggcgcagggtcgatgcgacgcaatcgtccgatccggagccgggactgtcgggcgtacacaaatcgcccgcagaagcg
cggccgtctggaccgatggctgtgtagaagtactcgccgatagtggaaaccgacgccccagcactcgtccgagggcaaaggaataggggga
tccgctgtaagtctgcagaaattgatgatctattaaacaataaagatgtccactaaaatggaagtttttcctgtcatactttgttaagaagggtgagaa
cagagtacctacattttgaatggaaggattggagctacgggggtgggggtggggtgggattagataaatgcctgctctttactgaaggctctttac
tattgctttatgataatgtttcatagttggatatcataatttaaacaagcaaaaccaaattaagggccagctcattcctcccactcatgatctatagat
ctatagatctctcgtgggatcattgtttttctcttgattcccactttgtggttctaagtactgtggtttccaaatgtgtcagtttcatagcctgaagaa
cgagatcagcagcctctgttccacatacacttcattctcagtattgttttgccaagttctaattccatcagacctcgacctgcagcccctagataactt
cgtataatgtatgctatacgaagttatgctagcGAGAGGTATCTGTGAAAGAAAGAAATGCTCATTAGACTTCCA
TTTTGTGTTCACTTATGTCCCTCAAAAGTATATTATCTTCATGGCTCTGATGTAACAA
Exemplary portion of a disrupted Mus musculus Kynu allele after recombinase-mediated
excision of a selection cassette (mouse sequence indicated in uppercase font and remaining
targeting vector sequence indicated in lowercase font; SEQ ID NO: 11):
TAATGGTGGACTCTGTAGAAGGCTGATATTCTGCAGAAAAAAAAATGATGATGGCTAC
ATTATTTCAACGTTTTACTTCCTTCTTAGATAACAGTTTATGggtaccgatttaaatgatccagtggtcctg
cagaggagagattgggagaatcccggtgtgacacagctgaacagactagccgcccaccctccctttgcttcttggagaaacagtgaggaagct
aggacagacagaccaagccagcaactcagatctttgaacggggagtggagatttgcctggtttccggcaccagaagcggtgccggaaagctg
gctggagtgcgatcttcctgaggccgatactgtcgtcgtcccctcaaactggcagatgcacggttacgatgcgcccatctacaccaacgtgacct
atcccattacggtcaatccgccgtttgttcccacggagaatccgacgggttgttactcgctcacatttaatgttgatgaaagctggctacaggaagg
ccagacgcgaattatttttgatggcgttaactcggcgtttcatctgtggtgcaacgggcgctgggtcggttacggccaggacagtcgtttgccgtct
gaatttgacctgagcgcatttttacgcgccggagaaaaccgcctcgcggtgatggtgctgcgctggagtgacggcagttatctggaagatcagg
atatgtggcggatgagcggcattttccgtgacgtctcgttgctgcataaaccgactacacaaatcagcgatttccatgttgccactcgctttaatgat
gatttcagccgcgctgtactggaggctgaagttcagatgtgcggcgagttgcgtgactacctacgggtaacagtttctttatggcagggtgaaac
gcaggtcgccagcggcaccgcgcctttcggcggtgaaattatcgatgagcgtggtggttatgccgatcgcgtcacactacgtctgaacgtcgaa
aacccgaaactgtggagcgccgaaatcccgaatctctatcgtgcggtggttgaactgcacaccgccgacggcacgctgattgaagcagaagc
ctgcgatgtcggtttccgcgaggtgcggattgaaaatggtctgctgctgctgaacggcaagccgttgctgattcgaggcgttaaccgtcacgagc
atcatcctctgcatggtcaggtcatggatgagcagacgatggtgcaggatatcctgctgatgaagcagaacaactttaacgccgtgcgctgttcg
cattatccgaaccatccgctgtggtacacgctgtgcgaccgctacggcctgtatgtggtggatgaagccaatattgaaacccacggcatggtgcc
aatgaatcgtctgaccgatgatccgcgctggctaccggcgatgagcgaacgcgtaacgcgaatggtgcagcgcgatcgtaatcacccgagtgt
gatcatctggtcgctggggaatgaatcaggccacggcgctaatcacgacgcgctgtatcgctggatcaaatctgtcgatccttcccgcccggtgc
agtatgaaggcggcggagccgacaccacggccaccgatattatttgcccgatgtacgcgcgcgtggatgaagaccagcccttcccggctgtg
ccgaaatggtccatcaaaaaatggctttcgctacctggagagacgcgcccgctgatcctttgcgaatacgcccacgcgatgggtaacagtcttg
gcggtttcgctaaatactggcaggcgtttcgtcagtatccccgtttacagggcggcttcgtctgggactgggtggatcagtcgctgattaaatatga
tgaaaacggcaacccgtggtcggcttacggcggtgattttggcgatacgccgaacgatcgccagttctgtatgaacggtctggtctttgccgacc
gcacgccgcatccagcgctgacggaagcaaaacaccagcagcagtttttccagttccgtttatccgggcaaaccatcgaagtgaccagcgaat
acctgttccgtcatagcgataacgagctcctgcactggatggtggcgctggatggtaagccgctggcaagcggtgaagtgcctctggatgtcgc
tccacaaggtaaacagttgattgaactgcctgaactaccgcagccggagagcgccgggcaactctggctcacagtacgcgtagtgcaaccgaa
cgcgaccgcatggtcagaagccgggcacatcagcgcctggcagcagtggcgtctggcggaaaacctcagtgtgacgctccccgccgcgtcc
cacgccatcccgcatctgaccaccagcgaaatggatttttgcatcgagctgggtaataagcgttggcaatttaaccgccagtcaggctttctttcac
agatgtggattggcgataaaaaacaactgctgacgccgctgcgcgatcagttcacccgtgcaccgctggataacgacattggcgtaagtgaag
cgacccgcattgaccctaacgcctgggtcgaacgctggaaggcggcgggccattaccaggccgaagcagcgttgttgcagtgcacggcaga
tacacttgctgatgcggtgctgattacgaccgctcacgcgtggcagcatcaggggaaaaccttatttatcagccggaaaacctaccggattgatg
gtagtggtcaaatggcgattaccgttgatgttgaagtggcgagcgatacaccgcatccggcgcggattggcctgaactgccagctggcgcagg
tagcagagcgggtaaactggctcggattagggccgcaagaaaactatcccgaccgccttactgccgcctgttttgaccgctgggatctgccattg
tcagacatgtataccccgtacgtcttcccgagcgaaaacggtctgcgctgcgggacgcgcgaattgaattatggcccacaccagtggcgcggc
gacttccagttcaacatcagccgctacagtcaacagcaactgatggaaaccagccatcgccatctgctgcacgcggaagaaggcacatggctg
aatatcgacggtttccatatggggattggtggcgacgactcctggagcccgtcagtatcggcggaattccagctgagcgccggtcgctaccatta
ccagttggtctggtgtcaaaaataataataaccgggcaggggggatctaagctctagataagtaatgatcataatcagccatatcacatctgtaga
ggttttacttgctttaaaaaacctcccacacctccccctgaacctgaaacataaaatgaatgcaattgttgttgttaacttgtttattgcagcttata
atggttacaaataaagcaatagcatcacaaatttcacaaataaagcatttttttcactgcattctagttgtggtttgtccaaactcatcaatgtatct
tatcatgtctggatcccccggctagagtttaaacactagaactagtggatccccgggctcgataactataacggtcctaaggtagcgactcgacataa
cttcgtataatgtatgctatacgaagttatgctagcGAGAGGTATCTGTGAAAGAAAGAAATGCTCATTAGACTTC
CATTTTGTGTTCACTTATGTCCCTCAAAAGTATATTATCTTCATGGCTCTGATGTAACAA
Exemplary portion of a mutant Mus musculus Kynu allele including a self-deleting
hygromycin selection cassette (mouse sequence indicated in regular uppercase font with mutated
nucleotides in bold and underlined text, and targeting vector sequence indicated in lowercase font;
SEQ ID NO: 12):
AGAGCCTGAGGCTTCTGTGGGAGTAACTGCAAGTTATTTATTACCCTTCCTCTTGTAAA
TTATGTTAATAACGCTGGATTAACAATGACAACTGGGAGAATGTTAATTAATTTAACAA
GCACTTTTTTTTTTGTATTTTCTTGTTTCAGTTGATCTATCTTTAGTGAGTGAGGATGATG
ATGCCATCTATTTCCTGGGAAATTCCCTTGGCCTTCAACCGAAAATGGTTAGGACATAC
CTGGAGGAAGAGCTTGAAAAATGGGCTAAGATGTAAGTACCAAGTTAAAAGGTGTAA
CTCCATCTGACAGAAGAATTCTGAAAATTACAAAATGTGTCTGATTTGGACAAGTTACA
CCCTAGCATATTAGGAACAATGAAAACCTTATTTACAGTAATTACCAATACTAAAATAT
TTTGATGAAATAATCTTCAATCAGAATAAGTCCAAATGACAAATTCATGAAAGctcgagata
acttcgtataatgtatgctatacgaagttatatgcatggcctccgcgccgggttttggcgcctcccgcgggcgcccccctcctcacggcgagcgc
tgccacgtcagacgaagggcgcagcgagcgtcctgatccttccgcccggacgctcaggacagcggcccgctgctcataagactcggccttag
aaccccagtatcagcagaaggacattttaggacgggacttgggtgactctagggcactggttttctttccagagagcggaacaggcgaggaaaa
gtagtcccttctcggcgattctgcggagggatctccgtggggcggtgaacgccgatgattatataaggacgcgccgggtgtggcacagctagtt
ccgtcgcagccgggatttgggtcgcggttcttgtttgtggatcgctgtgatcgtcacttggtgagtagcgggctgctgggctggccggggctttcg
tggccgccgggccgctcggtgggacggaagcgtgtggagagaccgccaagggctgtagtctgggtccgcgagcaaggttgccctgaactg
ggggttggggggagcgcagcaaaatggcggctgttcccgagtcttgaatggaagacgcttgtgaggcgggctgtgaggtcgttgaaacaagg
tggggggcatggtgggcggcaagaacccaaggtcttgaggccttcgctaatgcgggaaagctcttattcgggtgagatgggctggggcacca
tctggggaccctgacgtgaagtttgtcactgactggagaactcggtttgtcgtctgttgcgggggcggcagttatggcggtgccgttgggcagtg
cacccgtacctttgggagcgcgcgccctcgtcgtgtcgtgacgtcacccgttctgttggcttataatgcagggtggggccacctgccggtaggtg
tgcggtaggcttttctccgtcgcaggacgcagggttcgggcctagggtaggctctcctgaatcgacaggcgccggacctctggtgaggggag
ggataagtgaggcgtcagtttctttggtcggttttatgtacctatcttcttaagtagctgaagctccggttttgaactatgcgctcggggttggcgagt
gtgttttgtgaagttttttaggcaccttttgaaatgtaatcatttgggtcaatatgtaattttcagtgttagactagtaaattgtccgctaaattctgg
ccgtttttggcttttttgttagacgtgttgacaattaatcatcggcatagtatatcggcatagtataatacgacaaggtgaggaactaaaccatgaaaa
agcctgaactcaccgcgacgtctgtcgagaagtttctgatcgaaaagttcgacagcgtgtccgacctgatgcagctctcggagggcgaagaatctcgtg
ctttcagcttcgatgtaggagggcgtggatatgtcctgcgggtaaatagctgcgccgatggtttctacaaagatcgttatgtttatcggcactttgca
tcggccgcgctcccgattccggaagtgcttgacattggggaattcagcgagagcctgacctattgcatctcccgccgtgcacagggtgtcacgtt
gcaagacctgcctgaaaccgaactgcccgctgttctgcagccggtcgcggaggccatggatgcgattgctgcggccgatcttagccagacga
gcgggttcggcccattcggaccgcaaggaatcggtcaatacactacatggcgtgatttcatatgcgcgattgctgatccccatgtgtatcactggc
aaactgtgatggacgacaccgtcagtgcgtccgtcgcgcaggctctcgatgagctgatgctttgggccgaggactgccccgaagtccggcacc
tcgtgcacgcggatttcggctccaacaatgtcctgacggacaatggccgcataacagcggtcattgactggagcgaggcgatgttcggggattc
ccaatacgaggtcgccaacatcttcttctggaggccgtggttggcttgtatggagcagcagacgcgctacttcgagcggaggcatccggagctt
gcaggatcgccgcggctccgggcgtatatgctccgcattggtatgaccaactctatcagagatggttgacggcaatttcgatgatgcagatgg
gcgcagggtcgatgcgacgcaatcgtccgatccggagccgggactgtcgggcgtacacaaatcgcccgcagaagcgcggccgtctggacc
gatggctgtgtagaagtactcgccgatagtggaaaccgacgccccagcactcgtccgagggcaaaggaatagggggatccgctgtaagtctg
cagaaattgatgatctattaaacaataaagatgtccactaaaatggaagtttttcctgtcatactttgttaagaagggtgagaacagagtacctacatt
ttgaatggaaggattggagctacgggggtgggggtggggtgggattagataaatgcctgctctttactgaaggctctttactattgctttatgataat
gtttcatagttggatatcataatttaaacaagcaaaaccaaattaagggccagctcattcctcccactcatgatctatagatctatagatctctcgtgg
gatcattgtttttctcttgattcccactttgtggttctaagtactgtggtttccaaatgtgtcagtttcatagcctgaagaacgagatcagcagcctct
gttccacatacacttcattctcagtattgttttgccaagttctaattccatcagacctcgacctgcagcccctagcccgggcgccagtagcagcaccca
cgtccaccttctgtctagtaatgtccaacacctccctcagtccaaacactgctctgcatccatgtggctcccatttatacctgaagcacttgatggggc
ctcaatgttttactagagcccacccccctgcaactctgagaccctctggatttgtctgtcagtgcctcactggggcgttggataatttcttaaaaggtc
aagttccctcagcagcattctctgagcagtctgaagatgtgtgcttttcacagttcaaatccatgtggctgtttcacccacctgcctggccttgggtta
tctatcaggacctagcctagaagcaggtgtgtggcacttaacacctaagctgagtgactaactgaacactcaagtggatgccatctttgtcacttctt
gactgtgacacaagcaactcctgatgccaaagccctgcccacccctctcatgcccatatttggacatggtacaggtcctcactggccatggtctgt
gaggtcctggtcctctttgacttcataattcctaggggccactagtatctataagaggaagagggtgctggctcccaggccacagcccacaaaatt
ccacctgctcacaggttggctggctcgacccaggtggtgtcccctgctctgagccagctcccggccaagccagcaccatgggtacccccaaga
agaagaggaaggtgcgtaccgatttaaattccaatttactgaccgtacaccaaaatttgcctgcattaccggtcgatgcaacgagtgatgaggttc
gcaagaacctgatggacatgttcagggatcgccaggcgttttctgagcatacctggaaaatgcttctgtccgtttgccggtcgtgggcggcatggt
gcaagttgaataaccggaaatggtttcccgcagaacctgaagatgttcgcgattatcttctatatcttcaggcgcgcggtctggcagtaaaaactat
ccagcaacatttgggccagctaaacatgcttcatcgtcggtccgggctgccacgaccaagtgacagcaatgctgtttcactggttatgcggcgga
tccgaaaagaaaacgttgatgccggtgaacgtgcaaaacaggctctagcgttcgaacgcactgatttcgaccaggttcgttcactcatggaaaat
agtgatcgctgccaggatatacgtaatctggcatttctggggattgcttataacaccctgttacgtatagccgaaattgccaggatcagggttaaag
atatctcacgtactgacggtgggagaatgttaatccatattggcagaacgaaaacgctggttagcaccgcaggtgtagagaaggcacttagcctg
ggggtaactaaactggtcgagcgatggatttccgtctctggtgtagctgatgatccgaataactacctgttttgccgggtcagaaaaaatggtgttg
ccgcgccatctgccaccagccagctatcaactcgcgccctggaagggatttttgaagcaactcatcgattgatttacggcgctaaggtaaatataa
aatttttaagtgtataatgtgttaaactactgattctaattgtttgtgtattttaggatgactctggtcagagatacctggcctggtctggacacagt
gcccgtgtcggagccgcgcgagatatggcccgcgctggagtttcaataccggagatcatgcaagctggtggctggaccaatgtaaatattgtcatga
actatatccgtaacctggatagtgaaacaggggcaatggtgcgcctgctggaagatggcgattgatctagataagtaatgatcataatcagccata
tcacatctgtagaggttttacttgattaaaaaacctcccacacctccccctgaacctgaaacataaaatgaatgcaattgttgttgttaaacctgccct
agttgcggccaattccagctgagcgtgcctccgcaccattaccagttggtctggtgtcaaaaataataataaccgggcaggggggatctaagctc
tagataagtaatgatcataatcagccatatcacatctgtagaggttttacttgctttaaaaaacctcccacacctccccctgaacctgaaacataaaat
gaatgcaattgttgttgttaacttgtttattgcagcttataatggttacaaataaagcaatagcatcacaaatttcacaaataaagcatttttttcact
gcattctagttgtggtttgtccaaactcatcaatgtatcttatcatgtctggaataacttcgtataatgtatgctatacgaagttatgctagtaactat
aacggtcctaaggtagcgagctagcAGCCATTTAATGTCCAGCAAAGAAGTTAATTCATGATTTTGAGTGTTT
AATGATGAATTCATGACCAAGTTAAGAATGCCATCAAAAATAGGAAATACAG
Exemplary portion of a mutated Mus musculus Kynu allele including a self-deleting
neomycin selection cassette (mouse sequence indicated in regular uppercase font with mutated
nucleotides in bold and underlined text, and targeting vector sequence indicated in lowercase font;
SEQ ID NO: 13):
AGAGCCTGAGGCTTCTGTGGGAGTAACTGCAAGTTATTTATTACCCTTCCTCTTGTAAA
TTATGTTAATAACGCTGGATTAACAATGACAACTGGGAGAATGTTAATTAATTTAACAA
GCACTTTTTTTTTTGTATTTTCTTGTTTCAGTTGATCTATCTTTAGTGAGTGAGGATGATG
ATGCCATCTATTTCCTGGGAAATTCCCTTGGCCTTCAACCGAAAATGGTTAGGACATAC
CTGGAGGAAGAGCTTGAAAAATGGGCTAAGATGTAAGTACCAAGTTAAAAGGTGTAA
CTCCATCTGACAGAAGAATTCTGAAAATTACAAAATGTGTCTGATTTGGACAAGTTACA
CCCTAGCATATTAGGAACAATGAAAACCTTATTTACAGTAATTACCAATACTAAAATAT
TTTGATGAAATAATCTTCAATCAGAATAAGTCCAAATGACAAATTCATGAAAGctcgagata
acttcgtataatgtatgctatacgaagttatatgcatggcctccgcgccgggttttggcgcctcccgcgggcgcccccctcctcacggcgagcgc
tgccacgtcagacgaagggcgcagcgagcgtcctgatccttccgcccggacgctcaggacagcggcccgctgctcataagactcggccttag
aaccccagtatcagcagaaggacattttaggacgggacttgggtgactctagggcactggttttctttccagagagcggaacaggcgaggaaaa
gtagtcccttctcggcgattctgcggagggatctccgtggggcggtgaacgccgatgattatataaggacgcgccgggtgtggcacagctagtt
ccgtcgcagccgggatttgggtcgcggttcttgtttgtggatcgctgtgatcgtcacttggtgagtagcgggctgctgggctggccggggctttcg
tggccgccgggccgctcggtgggacggaagcgtgtggagagaccgccaagggctgtagtctgggtccgcgagcaaggttgccctgaactg
ggggttggggggagcgcagcaaaatggcggctgttcccgagtcttgaatggaagacgcttgtgaggcgggctgtgaggtcgttgaaacaagg
tggggggcatggtgggcggcaagaacccaaggtcttgaggccttcgctaatgcgggaaagctcttattcgggtgagatgggctggggcacca
tctggggaccctgacgtgaagtttgtcactgactggagaactcggtttgtcgtctgttgcgggggcggcagttatggcggtgccgttgggcagtg
cacccgtacctttgggagcgcgcgccctcgtcgtgtcgtgacgtcacccgttctgttggcttataatgcagggtggggccacctgccggtaggtg
tgcggtaggcttttctccgtcgcaggacgcagggttcgggcctagggtaggctctcctgaatcgacaggcgccggacctctggtgaggggag
ggataagtgaggcgtcagtttctttggtcggttttatgtacctatcttcttaagtagctgaagctccggttttgaactatgcgctcggggttggcgagt
gtgttttgtgaagttttttaggcaccttttgaaatgtaatcatttgggtcaatatgtaattttcagtgttagactagtaaattgtccgctaaattctgg
ccgtttttggcttttttgttagacgtgttgacaattaatcatcggcatagtatatcggcatagtataatacgacaaggtgaggaactaaaccatgggat
cggccattgaacaagatggattgcacgcaggttctccggccgcttgggtggagaggctattcggctatgactgggcacaacagacaatcggctgctct
gatgccgccgtgttccggctgtcagcgcaggggcgcccggttctttttgtcaagaccgacctgtccggtgccctgaatgaactgcaggacgagg
cagcgcggctatcgtggctggccacgacgggcgttccttgcgcagctgtgctcgacgttgtcactgaagcgggaagggactggctgctattgg
gcgaagtgccggggcaggatctcctgtcatctcaccttgctcctgccgagaaagtatccatcatggctgatgcaatgcggcggctgcatacgctt
gatccggctacctgcccattcgaccaccaagcgaaacatcgcatcgagcgagcacgtactcggatggaagccggtcttgtcgatcaggatgat
ctggacgaagagcatcaggggctcgcgccagccgaactgttcgccaggctcaaggcgcgcatgcccgacggcgatgatctcgtcgtgaccc
atggcgatgcctgcttgccgaatatcatggtggaaaatggccgcttttctggattcatcgactgtggccggctgggtgtggcggaccgctatcag
gacatagcgttggctacccgtgatattgctgaagagcttggcggcgaatgggctgaccgcttcctcgtgctttacggtatcgccgctcccgattcg
cagcgcatcgccttctatcgccttcttgacgagttcttctgaggggatccgctgtaagtctgcagaaattgatgatctattaaacaataaagatgtcc
actaaaatggaagtttttcctgtcatactttgttaagaagggtgagaacagagtacctacattttgaatggaaggattggagctacgggggtgggg
gtggggtgggattagataaatgcctgctattactgaaggctattactattgattatgataatgtttcatagttggatatcataatttaaacaagcaaa
accaaattaagggccagctcattcctcccactcatgatctatagatctatagatctctcgtgggatcattgtttttctcttgattcccactttgtggtt
ctaagtactgtggtttccaaatgtgtcagtttcatagcctgaagaacgagatcagcagcctctgttccacatacacttcattctcagtattgttttgcc
aagttctaattccatcagacctcgacctgcagcccctagcccgggcgccagtagcagcacccacgtccaccttctgtctagtaatgtccaacacctccc
tcagtccaaacactgctctgcatccatgtggctcccatttatacctgaagcacttgatggggcctcaatgttttactagagcccacccccctgcaact
ctgagaccctctggatttgtctgtcagtgcctcactggggcgttggataatttcttaaaaggtcaagttccctcagcagcattctctgagcagtctga
agatgtgtgcttttcacagttcaaatccatgtggctgtttcacccacctgcctggccttgggttatctatcaggacctagcctagaagcaggtgtgtg
gcacttaacacctaagctgagtgactaactgaacactcaagtggatgccatctttgtcacttcttgactgtgacacaagcaactcctgatgccaaag
ccctgcccacccctctcatgcccatatttggacatggtacaggtcctcactggccatggtctgtgaggtcctggtcctctttgacttcataattcctag
gggccactagtatctataagaggaagagggtgctggctcccaggccacagcccacaaaattccacctgctcacaggttggctggctcgaccca
ggtggtgtcccctgctctgagccagctcccggccaagccagcaccatgggtacccccaagaagaagaggaaggtgcgtaccgatttaaattcc
aatttactgaccgtacaccaaaatttgcctgcattaccggtcgatgcaacgagtgatgaggttcgcaagaacctgatggacatgttcagggatcgc
caggcgttttctgagcatacctggaaaatgcttctgtccgtttgccggtcgtgggcggcatggtgcaagttgaataaccggaaatggtttcccgca
gaacctgaagatgttcgcgattatcttctatatcttcaggcgcgcggtctggcagtaaaaactatccagcaacatttgggccagctaaacatgcttc
atcgtcggtccgggctgccacgaccaagtgacagcaatgctgtttcactggttatgcggcggatccgaaaagaaaacgttgatgccggtgaacg
tgcaaaacaggctctagcgttcgaacgcactgatttcgaccaggttcgttcactcatggaaaatagtgatcgctgccaggatatacgtaatctggc
atttctggggattgcttataacaccctgttacgtatagccgaaattgccaggatcagggttaaagatatctcacgtactgacggtgggagaatgtta
atccatattggcagaacgaaaacgctggttagcaccgcaggtgtagagaaggcacttagcctgggggtaactaaactggtcgagcgatggattt
ccgtctctggtgtagctgatgatccgaataactacctgttttgccgggtcagaaaaaatggtgttgccgcgccatctgccaccagccagctatcaa
ctcgcgccctggaagggatttttgaagcaactcatcgattgatttacggcgctaaggtaaatataaaatttttaagtgtataatgtgttaaactactg
attctaattgtttgtgtattttaggatgactctggtcagagatacctggcctggtctggacacagtgcccgtgtcggagccgcgcgagatatggcccg
cgctggagtttcaataccggagatcatgcaagctggtggctggaccaatgtaaatattgtcatgaactatatccgtaacctggatagtgaaacagg
ggcaatggtgcgcctgctggaagatggcgattgatctagataagtaatgatcataatcagccatatcacatctgtagaggttttacttgctttaaaaa
acctcccacacctccccctgaacctgaaacataaaatgaatgcaattgttgttgttaaacctgccctagttgcggccaattccagctgagcgtgcct
ccgcaccattaccagttggtctggtgtcaaaaataataataaccgggcaggggggatctaagctctagataagtaatgatcataatcagccatatc
acatctgtagaggttttacttgctttaaaaaacctcccacacctccccctgaacctgaaacataaaatgaatgcaattgttgttgttaacttgtttatt
gcagcttataatggttacaaataaagcaatagcatcacaaatttcacaaataaagcatttttttcactgcattctagttgtggtttgtccaaactcatc
aatgtatcttatcatgtctggaataacttcgtataatgtatgctatacgaagttatgctagtaactataacggtcctaaggtagcgagctagcAGCCAT
TTAATGTCCAGCAAAGAAGTTAATTCATGATTTTGAGTGTTTAATGATGAATTCATGACC
AAGTTAAGAATGCCATCAAAAATAGGAAATACAG
Exemplary portion of a mutant Mus musculus Kynu allele after recombinase-mediated
excision of a selection cassette (mouse sequence indicated in uppercase font with mutated
nucleotides in bold and underlined text, remaining 77 bp of targeting vector sequence after
recombinase-mediated deletion of a selection cassette indicated in lowercase font; SEQ ID NO: 14):
AGAGCCTGAGGCTTCTGTGGGAGTAACTGCAAGTTATTTATTACCCTTCCTCTTGTAAA
TTATGTTAATAACGCTGGATTAACAATGACAACTGGGAGAATGTTAATTAATTTAACAA
GCACTTTTTTTTTTGTATTTTCTTGTTTCAGTTGATCTATCTTTAGTGAGTGAGGATGATG
ATGCCATCTATTTCCTGGGAAATTCCCTTGGCCTTCAACCGAAAATGGTTAGGACATAC
CTGGAGGAAGAGCTTGAAAAATGGGCTAAGATGTAAGTACCAAGTTAAAAGGTGTAA
CTCCATCTGACAGAAGAATTCTGAAAATTACAAAATGTGTCTGATTTGGACAAGTTACA
CCCTAGCATATTAGGAACAATGAAAACCTTATTTACAGTAATTACCAATACTAAAATAT
TTTGATGAAATAATCTTCAATCAGAATAAGTCCAAATGACAAATTCATGAAAGctcgagata
acttcgtataatgtatgctatacgaagttatgctagtaactataacggtcctaaggtagcgagctagcAGCCATTTAATGTCCAGC
AAAGAAGTTAATTCATGATTTTGAGTGTTTAATGATGAATTCATGACCAAGTTAAGAAT
GCCATCAAAAATAGGAAATACAG

### DNA Constructs and Production of Engineered Non-Human Animals

Provided herein are DNA constructs or targeting vectors for the production of non-human animals having a disruption or mutation(s) in a Kynu gene as described herein.

DNA sequences can be used to prepare targeting vectors for knockout animals (e.g., an Kynu KO). Typically, a polynucleotide molecule (e.g., an insert nucleic acid) encoding a reporter gene or a mutant Kynu gene, in whole or in part, is inserted into a vector, preferably a DNA vector, in order to replicate the polynucleotide molecule in a suitable host cell.

A polynucleotide molecule (or insert nucleic acid) comprises a segment of DNA that one desires to integrate into a target locus or gene. In some embodiments, an insert nucleic acid comprises one or more polynucleotides of interest. In some embodiments, an insert nucleic acid comprises one or more expression cassettes. In some certain embodiments, an expression cassette comprises a polynucleotide of interest, a polynucleotide encoding a selection marker and/or a reporter gene along with, in some certain embodiments, various regulatory components that influence expression (e.g., promoter, enhancer, etc.). Virtually any polynucleotide of interest may be contained within an insert nucleic acid and thereby integrated at a target genomic locus. Methods disclosed herein, provide for at least 1, 2, 3, 4, 5, 6 or more polynucleotides of interest to be integrated into a targeted Kynu gene (or locus).

In some embodiments, a polynucleotide of interest contained in an insert nucleic acid encodes a reporter. In some embodiments, a polynucleotide of interest contained in an insert nucleic acid encodes a selectable marker and/or a recombinase.

In some embodiments, a polynucleotide of interest is flanked by or comprises site-specific recombination sites (e.g., loxP, Frt, etc.). In some certain embodiments, site-specific recombination sites flank a DNA segment that encodes a reporter, a DNA segment that encodes a selectable marker, a DNA segment that encodes a recombinase, and combinations thereof. Exemplary polynucleotides of interest, including selection markers, reporter genes and recombinase genes that can be included within insert nucleic acids are described herein.

Depending on size, a Kynu gene or Kynu-encoding sequence as can be cloned directly from cDNA sources available from commercial suppliers or designed in silico based on published sequences available from GenBank (see above). Alternatively, bacterial artificial chromosome (BAC) libraries can provide Kynu sequences from genes of interest (e.g., a rodent or heterologous Kynu gene). BAC libraries contain an average insert size of 100-150 kb and are capable of harboring inserts as large as 300 kb (Shizuya, H. et al., 1992, Proc. Natl. Acad. Sci., U.S.A. 89:8794-7; Swiatek, P. J. and T. Gridley, 1993, Genes Dev. 7:2071-84; Kim, U. J. et al., 1996, Genomics 34:213-8; herein incorporated by reference). For example, human and mouse genomic BAC libraries have been constructed and are commercially available (e.g., Invitrogen, Carlsbad Calif). Genomic BAC libraries can also serve as a source of rodent or heterologous Kynu sequences as well as transcriptional control regions.

Alternatively, rodent or heterologous Kynu sequences may be isolated, cloned and/or transferred from yeast artificial chromosomes (YACs). An entire rodent or heterologous Kynu gene can be cloned and contained within one or a few YACs. If multiple YACs are employed and contain regions of overlapping homology, they can be recombined within yeast host strains to produce a single construct representing the entire locus. YAC arms can be additionally modified with mammalian selection cassettes by retrofitting to assist in introducing the constructs into embryonic stems cells or embryos by methods known in the art and/or described herein.

DNA constructs or targeting vectors containing Kynu sequences as described herein, in some embodiments, comprise rodent Kynu genomic sequences encoding a rodent Kynu polypeptide that includes one or more amino acid substitutions as compared to a wild-type or parent rodent Kynu polypeptide operably linked to non-human regulatory sequences (e.g., a rodent promoter) for expression in a transgenic non-human animal. In some embodiments, DNA constructs or targeting vectors containing Kynu sequences as described herein comprise rodent Kynu genomic sequences encoding a variant rodent Kynu polypeptide that includes a D93E substitution as compared to a wild-type or parent rodent Kynu polypeptide operably linked to a rodent Kynu promoter. Rodent and/or heterologous sequences included in DNA constructs described herein may be identical or substantially identical with rodent and/or heterologous sequences found in nature (e.g., genomic). Alternatively, such sequences may be artificial (e.g., synthetic) or may be engineered by the hand of man. In some embodiments, Kynu sequences are synthetic in origin and include a sequence or sequences that are found in a rodent or heterologous Kynu gene found in nature. In some embodiments, Kynu sequences comprise a sequence naturally associated with a rodent or heterologous Kynu gene. In some embodiments, Kynu sequences comprise a sequence that is not naturally associated with a rodent or heterologous Kynu gene. In some embodiments, Kynu sequences comprise a sequence that is optimized for expression in a non-human animal. If additional sequences are useful in optimizing expression of a mutant Kynu gene described herein, such sequences can be cloned using existing sequences as probes. Additional sequences necessary for maximizing expression of a mutant Kynu gene or Kynu-encoding sequence can be obtained from genomic sequences or other sources depending on the desired outcome.

DNA constructs or targeting vectors can be prepared using methods known in the art. For example, a DNA construct can be prepared as part of a larger plasmid. Such preparation allows the cloning and selection of the correct constructions in an efficient manner as is known in the art. DNA fragments containing sequences as described herein can be located between convenient restriction sites on the plasmid so that they can be easily isolated from the remaining plasmid sequences for incorporation into the desired animal.

Various methods employed in preparation of plasmids, DNA constructs and/or targeting vectors and transformation of host organisms are known in the art. For other suitable expression systems for both prokaryotic and eukaryotic cells, as well as general recombinant procedures, see Molecular Cloning: A Laboratory Manual, 2nd Ed., ed. by Sambrook, J. et al., Cold Spring Harbor Laboratory Press: 1989.

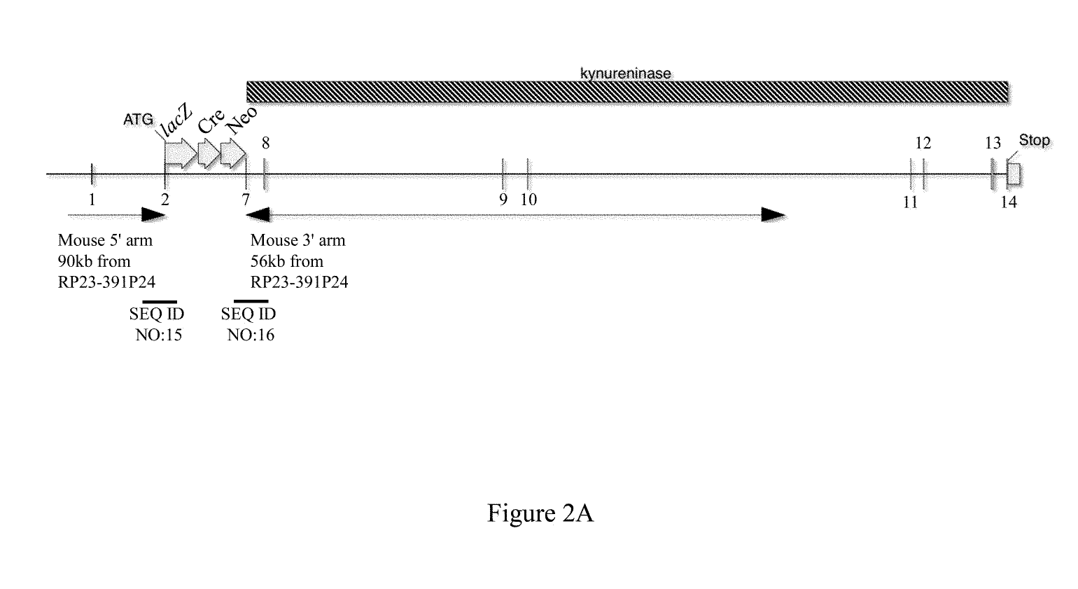

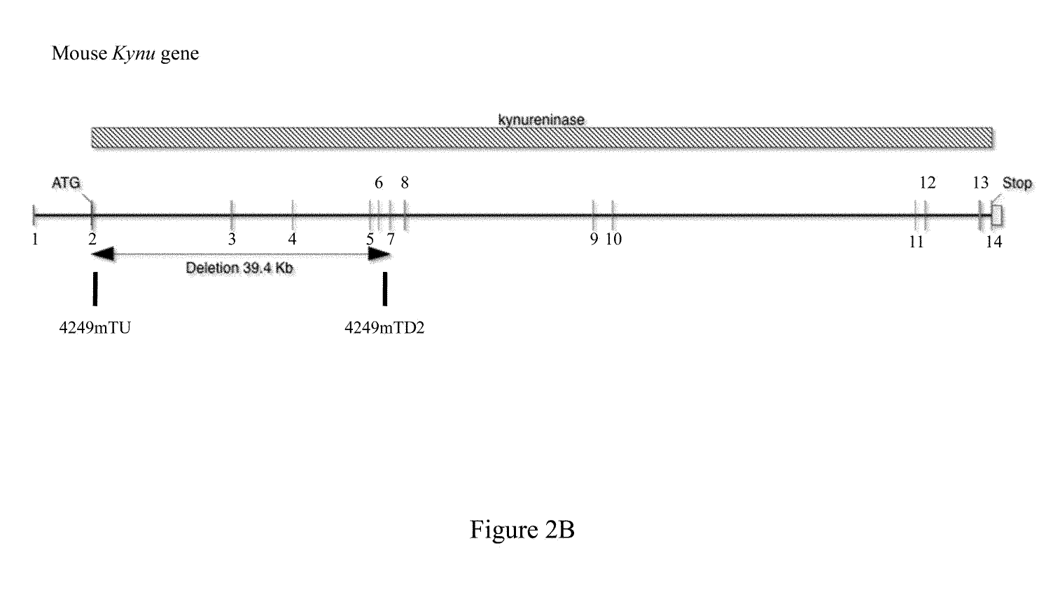

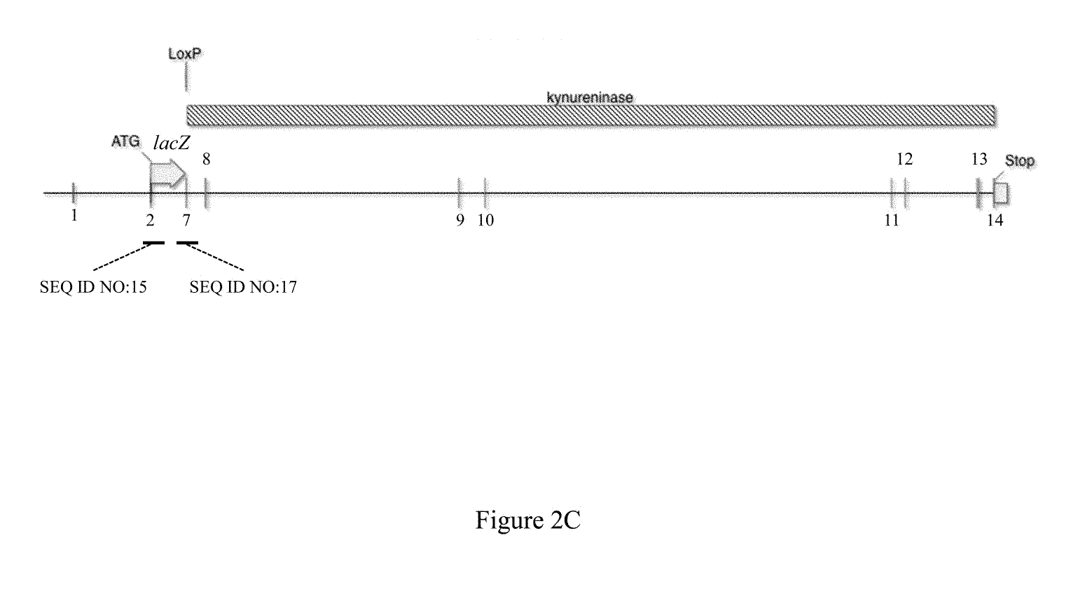

As described above, exemplary non-human (e.g., rodent) Kynu nucleic acid and amino acid sequences for use in constructing targeting vectors for non-human animals containing a disrupted or mutant Kynu gene are provided above. Other non-human Kynu sequences can also be found in the GenBank database. Kynu targeting vectors, in some embodiments, comprise DNA sequences encoding a reporter gene, a selectable marker, a recombinase gene (or combinations thereof) and non-human Kynu sequences (i.e., flanking sequences of a target region) for insertion into the genome of a transgenic non-human animal. In one example, a deletion start point may be set of immediately downstream (3′) of a start codon in a first coding exon to allow an insert nucleic acid to be operably linked to an endogenous regulatory sequence (e.g., a promoter). FIGS. 2A-2C illustrate an exemplary targeting vector for making a targeted deletion of a portion of the coding sequence (e.g., exons 2-6) a murine Kynu gene, excluding the start codon, and replacement with a cassette that contains a sequence from a lacZ gene that encodes (3-galactosidase and a drug selection cassette that encodes neomycin phosphotransferase (Neo) for the selection of G418-resistant embryonic stem (ES) cell colonies. The targeting vector also includes a sequence encoding a recombinase (e.g., Cre) regulated by an ES-cell specific micro RNAs (miRNAs) or a germ-cell specific promoter (e.g., protamine 1 promoter; Prot-Cre-SV40). The neomycin selection cassette and Cre recombinase-encoding sequences are flanked by loxP recombinase recognition sites that enable Cre-mediated excision of the neomycin selection cassette in a development-dependent manner, i.e., progeny derived from rodents whose germ cells contain the disrupted Kynu gene described above will shed the selectable marker during development (see U.S. Pat. Nos. 8,697,851, 8,518,392, 8,354,389, 8,946,505, and 8,946,504, all of which are herein incorporated by reference). This allows for, among other things, automatic excision of the neomycin selection cassette from either differentiated cells or germ cells. Thus, prior to phenotypic analysis the neomycin selection cassette is removed leaving only the lacZ reporter gene (fused to the mouse Kynu start codon) operably linked to the murine Kynu promoter (FIG. 2C).

As described herein, disruption of a Kynu gene can comprise a replacement of or an insertion/addition to the Kynu gene or a portion thereof with an insert nucleic acid. In some embodiments, an insert nucleic acid comprises a reporter gene. In some certain embodiments, a reporter gene is positioned in operable linkage with an endogenous Kynu promoter. Such a modification allows for the expression of a reporter gene driven by an endogenous Kynu promoter. Alternatively, a reporter gene is not placed in operable linkage with an endogenous Kynu promoter.

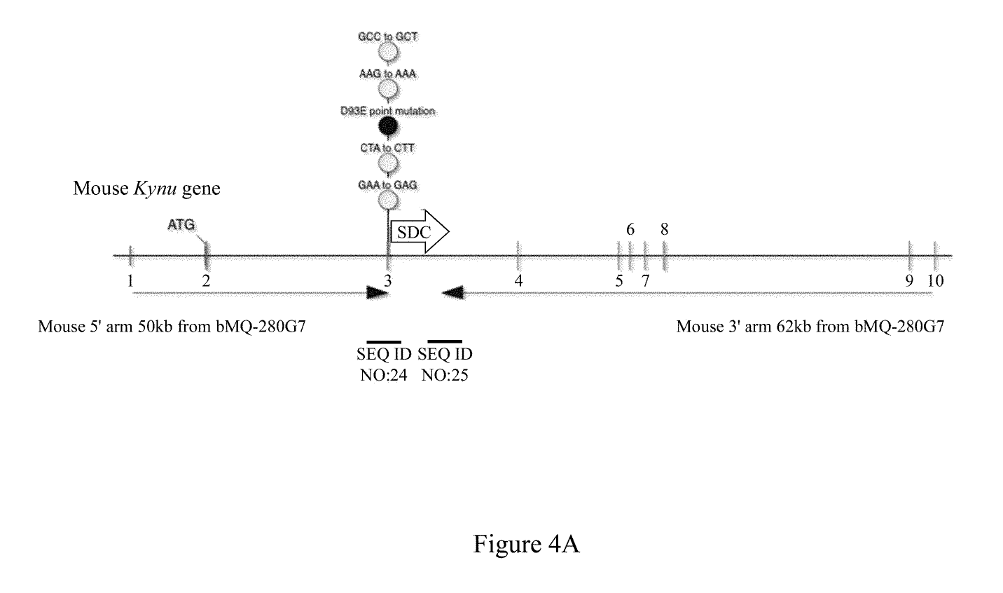

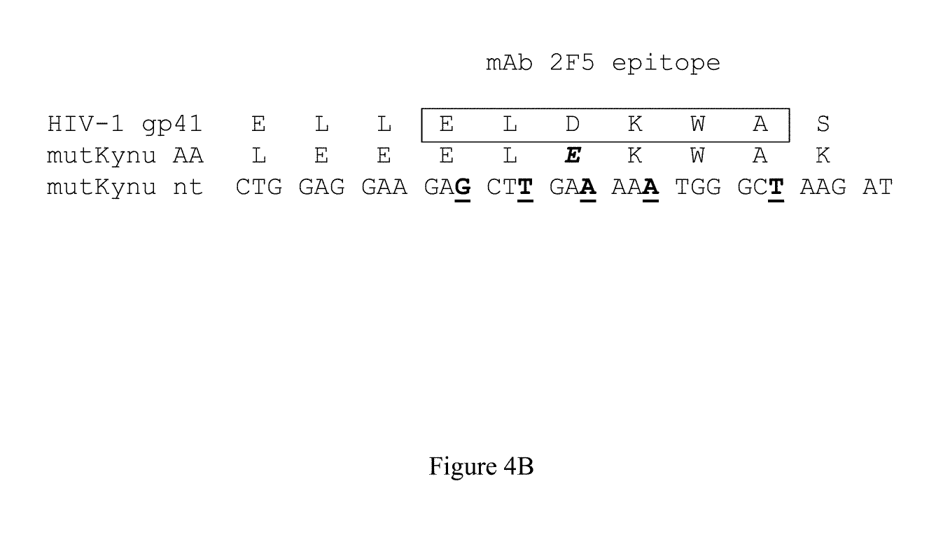

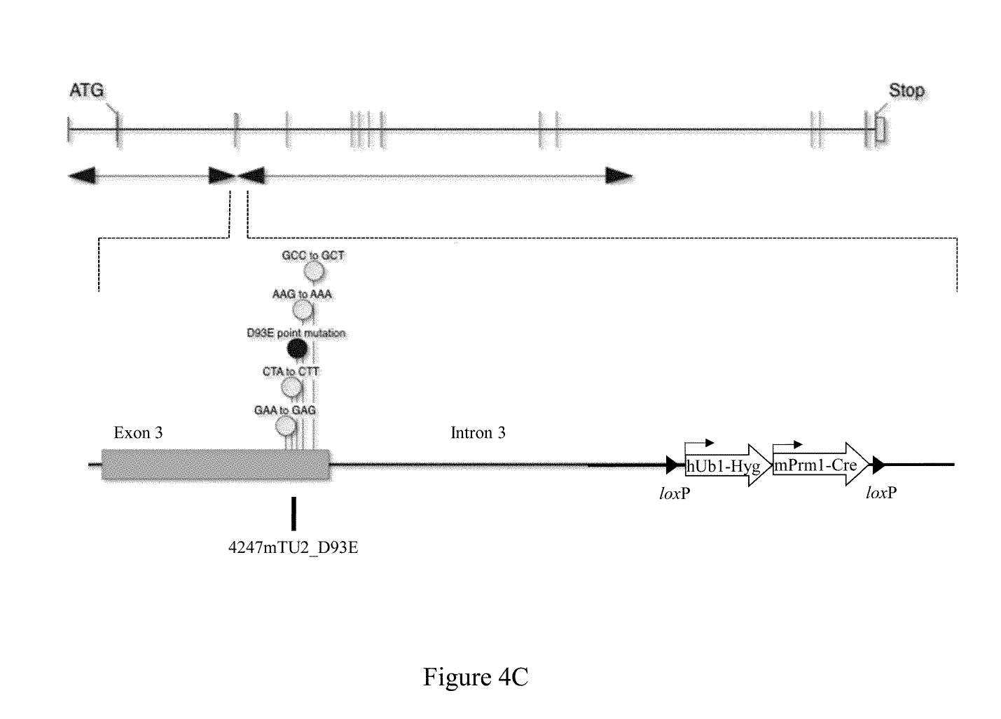

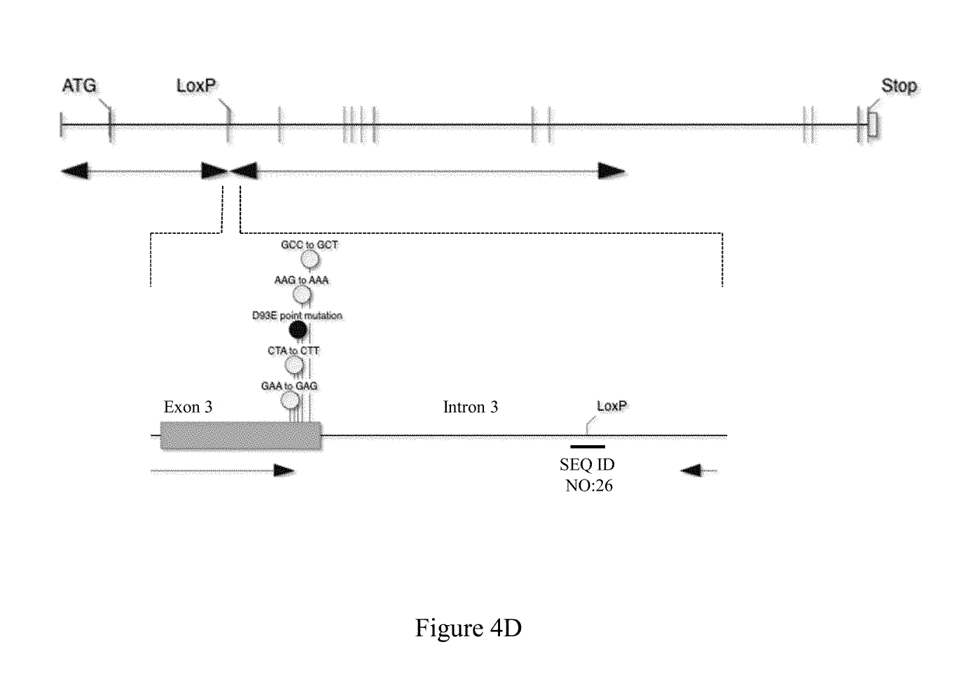

A variety of reporter genes (or detectable moieties) can be used in targeting vectors described herein. Exemplary reporter genes include, for example, β-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting vectors that employ the use of a lacZ reporter gene that encodes β-galactosidase, however, persons of skill upon reading this disclosure will understand that non-human animals described herein can be generated in the absence of a reporter gene or with any reporter gene known in the art. Kynu targeting vectors, in some embodiments, comprise DNA sequences encoding a mutant Kynu gene, a selectable marker and a recombinase, and non-human Kynu sequences (i.e., flanking sequences of a target region) for insertion into the genome of a transgenic non-human animal. In one example, one or more point mutations may be introduced (e.g., by site-directed mutagenesis) into the coding sequence of a Kynu gene or Kynu-encoding sequence (e.g., an exon) so that a desired Kynu polypeptide (e.g., a variant Kynu polypeptide) is encoded by the mutant Kynu gene or Kynu-encoding sequence. Such a mutant Kynu sequence may be operably linked to an endogenous regulatory sequence (e.g., a promoter) or constitutive promoter as desired. FIGS. 4A and 4C illustrate an exemplary targeting vector for making one or more point mutations in an exon (e.g., exon three) of a murine Kynu gene and a small deletion in intron three with a cassette that contains a drug selection marker that encodes hygromycin (Hyg) for the selection of mutant embryonic stem (ES) cell colonies. As described in the examples section, several of the point mutations introduced into mouse Kynu exon three, and the deletion in intron three, were designed to facilitate screening of mutant ES cell colonies. As shown in FIG. 4C, the targeting vector also includes a sequence encoding a recombinase (e.g., Cre) regulated by an ES-cell specific miRNAs or a germ-cell specific promoter (e.g., protamine 1 promoter; Prot-Cre-SV40). The hygromycin selection cassette and Cre recombinase-encoding sequences are flanked by loxP recombinase recognition sites that enable Cre-mediated excision of the hygromycin selection cassette in a development-dependent manner, e.g., progeny derived from rodents whose germ cells containing the mutant Kynu gene described above will shed the selectable marker during development (see U.S. Pat. Nos. 8,697,851, 8,518,392, 8,354,389, 8,946,505, and 8,946,504, all of which are herein incorporated by reference). This allows for, among other things, automatic excision of the hygromycin selection cassette from either differentiated cells or germ cells. Thus, prior to phenotypic analysis the hygromycin selection cassette is removed leaving the mutant Kynu exon three (and a loxP site in intron three) operably linked to the murine Kynu promoter (FIG. 4D).

Where appropriate, the coding region of the genetic material or polynucleotide sequence(s) encoding a reporter polypeptide (and/or a selectable marker, and/or a recombinase), in whole or in part, or a Kynu polypeptide (e.g., a variant Kynu polypeptide) may be modified to include codons that are optimized for expression in the non-human animal (e.g., see U.S. Pat. Nos. 5,670,356 and 5,874,304). Codon optimized sequences are synthetic sequences, and preferably encode the identical polypeptide (or a biologically active fragment of a full length polypeptide which has substantially the same activity as the full length polypeptide) encoded by the non-codon optimized parent polynucleotide. In some embodiments, the coding region of the genetic material encoding a reporter polypeptide (e.g., lacZ), in whole or in part, may include an altered sequence to optimize codon usage for a particular cell type (e.g., a rodent cell). In some embodiments, the coding region of the genetic material encoding a Kynu polypeptide as described herein (e.g., a variant Kynu polypeptide), in whole or in part, may include an altered sequence to optimize codon usage for a particular cell type (e.g., a rodent cell). To give but one example, the codons of the reporter or mutant Kynu gene to be inserted into the genome of a non-human animal (e.g., a rodent) may be optimized for expression in a cell of the non-human animal. Such a sequence may be described as a codon-optimized sequence.

Compositions and methods for making non-human animals that comprise a disruption or mutation in a Kynu gene as described herein are provided, including compositions and methods for making non-human animals that express a reporter gene from a Kynu promoter and a Kynu regulatory sequence, and non-human animals that express a variant Kynu polypeptide from a Kynu promoter and a Kynu regulatory sequence. In some embodiments, compositions and methods for making non-human animals that express a reporter gene or a variant Kynu polypeptide from an endogenous promoter and an endogenous regulatory sequence are also provided. Methods include inserting a targeting vector, as described herein, encoding a reporter gene (e.g., lacZ; see FIGS. 2A-2C), in whole or in part, into the genome of a non-human animal so that a portion of the coding sequence of a Kynu gene is deleted, in whole or in part. In some embodiments, methods include inserting a targeting vector into the genome of a non-human animal so that exons 2-6 of a Kynu gene are deleted.

Insertion of a reporter gene operably linked to a Kynu promoter (e.g., an endogenous Kynu promoter) employs a relatively minimal modification of the genome and results in expression of reporter polypeptide in a Kynu-specific manner in the non-human animal. In some embodiments, a non-human animal or cell as described herein comprises a Kynu gene that comprises a targeting vector as described herein; in some certain embodiments, a targeting vector that appears in FIG. 2A or 2C.

In various embodiments, a disrupted Kynu gene as described herein includes one or more (e.g., first and second) insertion junctions resulting from insertion of a reporter gene.

In various embodiments, a disrupted Kynu gene as described herein includes a first insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:15 and a second insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:16.

In various embodiments, a disrupted Kynu gene as described herein includes a first insertion junction that includes a sequence that is substantially identical or identical to SEQ ID NO:15 and a second insertion junction that includes a sequence that is substantially identical or identical to SEQ ID NO:16.

In various embodiments, a disrupted Kynu gene as described herein includes a first insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:15 and a second insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:17.

In various embodiments, a disrupted Kynu gene as described herein includes a first insertion junction that includes a sequence that is substantially identical or identical to SEQ ID NO:15 and a second insertion junction that includes a sequence that is substantially identical or identical to SEQ ID NO:17.

In various embodiments, a disrupted Kynu gene or allele as described herein includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:9, SEQ ID NO: 10 or SEQ ID NO:11.

In various embodiments, a disrupted Kynu gene or allele as described herein includes a sequence that is substantially identical or identical to SEQ ID NO:9, SEQ ID NO: 10 or SEQ ID NO:11.

Methods also include inserting a targeting vector, as described herein, encoding a variant Kynu polypeptide (see FIGS. 4A-4D), in whole or in part, into the genome of a non-human animal so that a portion (e.g., exon three) of the coding sequence of a Kynu gene is altered. In some embodiments, methods include inserting targeting vector into the genome of a non-human animal so that exon three of a Kynu gene is mutated to encode a variant Kynu polypeptide.

Insertion of a mutant Kynu gene operably linked to a Kynu promoter (e.g., an endogenous Kynu promoter) employs a relatively minimal modification of the genome and results in expression of variant Kynu polypeptide in the non-human animal that is functionally and structurally similar to a Kynu polypeptide that appears in a wild-type non-human animal. In some embodiments, a non-human animal or cell described herein comprises a Kynu gene that comprises a targeting vector as described herein; in some certain embodiments, a targeting vector that appears in FIG. 4A, 4C or 4D.

In various embodiments, a mutant Kynu gene as described herein includes one or more (e.g., first and second) insertion junctions resulting from insertion of a targeting vector as described herein.

In various embodiments, a mutant Kynu gene as described herein includes a first insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:24 and a second insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:25.

In various embodiments, a mutant Kynu gene as described herein includes a first insertion junction that includes a sequence that is substantially identical or identical to SEQ ID NO:24 and a second insertion junction that includes a sequence that is substantially identical or identical to SEQ ID NO:25.

In various embodiments, a mutant Kynu gene as described herein includes an insertion junction that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:26.

In various embodiments, a mutant Kynu gene as described herein includes an insertion junction that includes a sequence that is substantially identical or identical to SEQ ID NO:26.

In various embodiments, a mutant Kynu gene as described herein comprises a third exon that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:42.

In various embodiments, a mutant Kynu gene as described herein comprises a third exon that includes a sequence that is substantially identical or identical to SEQ ID NO:42.

In various embodiments, a mutant Kynu gene as described herein comprises a third intron that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:26.

In various embodiments, a mutant Kynu gene as described herein comprises a third intron that includes a sequence that is substantially identical or identical to SEQ ID NO:26.

In various embodiments, a mutant Kynu gene as described herein comprises a third exon that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:42, and comprises a third intron that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:26.

In various embodiments, a mutant Kynu gene as described herein comprises a third exon that includes a sequence that is substantially identical or identical to SEQ ID NO:42, and comprises a third intron that includes a sequence that is substantially identical or identical to SEQ ID NO:26.

In various embodiments, a mutant Kynu gene or allele as described herein comprises a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:12, SEQ ID NO: 13 or SEQ ID NO:14.

In various embodiments, a mutant Kynu gene or allele as described herein comprises a sequence that is substantially identical or identical to SEQ ID NO:12, SEQ ID NO: 13 or SEQ ID NO:14.

In various embodiments, a mutant Kynu gene in a non-human animal described herein encodes an mRNA sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:7.

In various embodiments, a mutant Kynu gene in a non-human animal described herein encodes an mRNA sequence that is substantially identical or identical to SEQ ID NO:7.

In various embodiments, a mutant Kynu gene in a non-human animal described herein comprises a third exon that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:42.

In various embodiments, a mutant Kynu gene in a non-human animal described herein comprises a third exon that includes a sequence that is substantially identical or identical to SEQ ID NO:42.

In various embodiments, a Kynu polypeptide produced or expressed by a non-human animal described herein comprises a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:8.

In various embodiments, a Kynu polypeptide produced or expressed by a non-human animal described herein comprises a sequence that is substantially identical or identical to SEQ ID NO:8.

In various embodiments, a Kynu polypeptide produced or expressed by a non-human animal described herein comprises an H4 domain that includes a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to SEQ ID NO:41 or SEQ ID NO:36.

In various embodiments, a Kynu polypeptide produced or expressed by a non-human animal described herein comprises an H4 domain that includes a sequence that is substantially identical or identical to SEQ ID NO:41 or SEQ ID NO:36.

Alternatively, other Kynu genes or Kynu-encoding sequences may be employed in the methods described herein to generate non-human animals whose genomes contain a mutant Kynu gene as described herein. For example, a heterologous Kynu gene may be introduced into a non-human animal, which heterologous Kynu gene encodes a variant Kynu polypeptide as described herein (i.e., lacks a shared epitope with the MPER of HIV-1 gp41). In another example, a transgenic Kynu gene may be randomly inserted into the genome a non-human animal and an endogenous Kynu gene rendered non-functional (e.g., via genetic modification, gene knockdown with DNA or RNA oligonucleotides, etc.). Exemplary alternative Kynu genes or Kynu-encoding sequences include any Kynu gene or Kynu-encoding sequence (e.g., engineered) that encode a polypeptide that lacks one or more epitopes that is shared with or present in an HIV envelope polypeptide. To give but one example, Kynu genes in other species that encode Kynu polypeptides that do not contain epitopes that are shared with or present in an HIV envelope are known in the art. Persons of skill upon reading this disclosure will understand that such Kynu genes or Kynu-encoding sequences can be employed in the methods described herein to generate non-human animals.

Targeting vectors described herein may be introduced into ES cells and screened for ES clones harboring a disrupted or mutant Kynu gene as described herein in Frendewey, D., et al., 2010, Methods Enzymol. 476:295-307. A variety of host embryos can be employed in the methods and compositions disclosed herein. For example, the pluripotent and/or totipotent cells having the targeted genetic modification can be introduced into a pre-morula stage embryo (e.g., an 8-cell stage embryo) from a corresponding organism. See, e.g., U.S. Pat. Nos. 7,576,259, 7,659,442, 7,294,754, and U.S. Patent Application Publication No. 2008-0078000 A1, all of which are incorporated herein by reference in their entireties. In other instances, donor ES cells may be implanted into a host embryo at the 2-cell stage, 4-cell stage, 8-cell stage, 16-cell stage, 32-cell stage, or 64-cell stage. A host embryo can also be a blastocyst or can be a pre-blastocyst embryo, a pre-morula stage embryo, a morula stage embryo, an uncompacted morula stage embryo, or a compacted morula stage embryo.

In some embodiments, the VELOCIMOUSE® method (Poueymirou, W. T. et al., 2007, Nat. Biotechnol. 25:91-99) may be applied to inject positive ES cells into an 8-cell embryo to generate fully ES cell-derived F0 generation heterozygous mice ready for lacZ expression profiling or breeding to homozygosity. Exemplary methods for generating non-human animals having a disrupted or mutant Kynu gene are provided in the Example section.

Methods for generating transgenic non-human animals, including knockouts and knock-ins, are well known in the art (see, e.g., Kitamura, D. et al., 1991, Nature 350:423-6; Komori, T. et al., 1993, Science 261:1171-5; Shinkai, Y. et al., 1993, Science 259:822-5; Mansour, S. L. et al., 1998, Nature 336:348-52; Gene Targeting: A Practical Approach, Joyner, ed., Oxford University Press, Inc., 2000; Valenzuela, D. M. et al., 2003, Nature Biotech. 21(6):652-9; Adams, N.C. and N. W. Gale, in Mammalian and Avian Transgenesis-New Approaches, ed. Lois, S.P.a.C., Springer Verlag, Berlin Heidelberg, 2006). For example, generation of transgenic rodents may involve disruption of the genetic loci of an endogenous rodent gene and introduction of a reporter gene into the rodent genome, in some embodiments, at the same location as the endogenous rodent gene, or may involve the altering the genetic loci of an endogenous rodent gene and introduction of one or more mutations into the rodent genome, in some embodiments, at the same location as the endogenous rodent gene, resulting in the expression of a variant polypeptide.

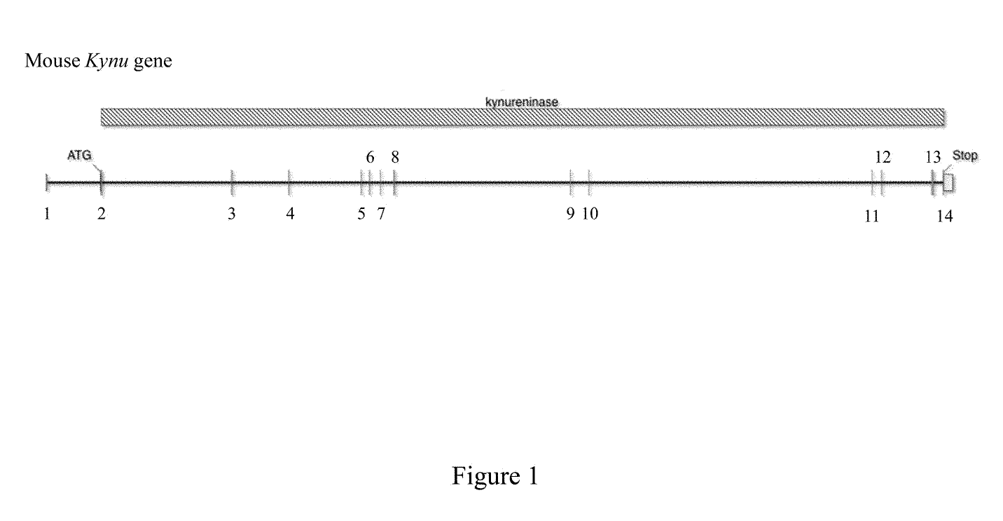

A schematic illustration (not to scale) of the genomic organization of a mouse Kynu gene is provided in FIG. 1. An exemplary targeting vector for deletion of a portion of the coding sequence of mouse Kynu gene using a reporter gene is provided in FIG. 2A. As illustrated, genomic DNA containing exons 2-6 (with the exception of the ATG start codon in exon 2) of a mouse Kynu gene is deleted with a reporter gene and a self-deleting drug selection cassette flanked by site-specific recombinase recognition sites. The targeting vector includes a recombinase-encoding sequence that is operably linked to a promoter that is developmentally regulated such that the recombinase is expressed in undifferentiated cells. Upon homologous recombination, exons 2-6 of an endogenous mouse Kynu gene are deleted (or replaced) by the sequence contained in the targeting vector as shown and engineered mice having a Kynu gene that has the structure depicted in FIG. 2C are created via Cre-mediated excision of the neomycin cassette during development leaving the lacZ reporter gene (fused to a mouse Kynu start codon) operably linked to the mouse Kynu promoter.

An exemplary targeting vector for creating a mutation in mouse Kynu gene is provided in FIGS. 4A and 4C. As illustrated, a mutant mouse Kynu gene (i.e., a mutant Kynu gene having an exon three that includes point mutations) is created with a targeting vector that includes a self-deleting drug selection cassette flanked by site-specific recombinase recognition sites placed downstream of a mutant Kynu exon three and within a Kynu intron three (see also FIG. 4C). The targeting vector includes a recombinase-encoding sequence that is operably linked to a promoter that is developmentally regulated such that the recombinase is expressed in undifferentiated cells. Upon homologous recombination, exon three (and intron three) of an endogenous mouse Kynu gene is replaced by the sequence contained in the targeting vector as shown and engineered mice having a mutant Kynu gene that has the structure depicted in FIG. 4D are created via Cre-mediated excision of the selection cassette during development leaving a mutant Kynu gene having one or more point mutations in exon three operably linked to a mouse Kynu promoter, and a small deletion (with a unique loxP site) within intron three. The resulting mutant Kynu gene encodes a Kynu polypeptide that includes a D93E amino acid substitution (see FIG. 4C for portion of exon three encoding D93E substitution).

Exemplary promoters than can be included in targeting vectors described herein are provided below. Additional suitable promoters that can be used in targeting vectors described herein include those described in U.S. Pat. Nos. 8,697,851, 8,518,392 and 8,354,389; all of which are incorporated herein by reference).

Protamine 1 (Prm1) promoter (SEQ ID NO: 37):
CCAGTAGCAGCACCCACGTCCACCTTCTGTCTAGTAATGTCCAACACCTC
CCTCAGTCCAAACACTGCTCTGCATCCATGTGGCTCCCATTTATACCTGA
AGCACTTGATGGGGCCTCAATGTTTTACTAGAGCCCACCCCCCTGCAACT
CTGAGACCCTCTGGATTTGTCTGTCAGTGCCTCACTGGGGCGTTGGATAA
TTTCTTAAAAGGTCAAGTTCCCTCAGCAGCATTCTCTGAGCAGTCTGAAG
ATGTGTGCTTTTCACAGTTCAAATCCATGTGGCTGTTTCACCCACCTGCC
TGGCCTTGGGTTATCTATCAGGACCTAGCCTAGAAGCAGGTGTGTGGCAC
TTAACACCTAAGCTGAGTGACTAACTGAACACTCAAGTGGATGCCATCTT
TGTCACTTCTTGACTGTGACACAAGCAACTCCTGATGCCAAAGCCCTGCC
CACCCCTCTCATGCCCATATTTGGACATGGTACAGGTCCTCACTGGCCAT
GGTCTGTGAGGTCCTGGTCCTCTTTGACTTCATAATTCCTAGGGGCCACT
AGTATCTATAAGAGGAAGAGGGTGCTGGCTCCCAGGCCACAGCCCACAAA
ATTCCACCTGCTCACAGGTTGGCTGGCTCGACCCAGGTGGTGTCCCCTGC
TCTGAGCCAGCTCCCGGCCAAGCCAGCACC
Blimp 1 promoter 1 kb (SEQ ID NO: 38):
TGCCATCATCACAGGATGTCCTTCCTTCTCCAGAAGACAGACTGGGGCTG
AAGGAAAAGCCGGCCAGGCTCAGAACGAGCCCCACTAATTACTGCCTCCA
ACAGCTTTCCACTCACTGCCCCCAGCCCAACATCCCCTTTTTAACTGGGA
AGCATTCCTACTCTCCATTGTACGCACACGCTCGGAAGCCTGGCTGTGGG
TTTGGGCATGAGAGGCAGGGACAACAAAACCAGTATATATGATTATAACT
TTTTCCTGTTTCCCTATTTCCAAATGGTCGAAAGGAGGAAGTTAGGTCTA
CCTAAGCTGAATGTATTCAGTTAGCAGGAGAAATGAAATCCTATACGTTT
AATACTAGAGGAGAACCGCCTTAGAATATTTATTTCATTGGCAATGACTC
CAGGACTACACAGCGAAATTGTATTGCATGTGCTGCCAAAATACTTTAGC
TCTTTCCTTCGAAGTACGTCGGATCCTGTAATTGAGACACCGAGTTTAGG
TGACTAGGGTTTTCTTTTGAGGAGGAGTCCCCCACCCCGCCCCGCTCTGC
CGCGACAGGAAGCTAGCGATCCGGAGGACTTAGAATACAATCGTAGTGTG
GGTAAACATGGAGGGCAAGCGCCTGCAAAGGGAAGTAAGAAGATTCCCAG
TCCTTGTTGAAATCCATTTGCAAACAGAGGAAGCTGCCGCGGGTCGCAGT
CGGTGGGGGGAAGCCCTGAACCCCACGCTGCACGGCTGGGCTGGCCAGGT
GCGGCCACGCCCCCATCGCGGCGGCTGGTAGGAGTGAATCAGACCGTCAG
TATTGGTAAAGAAGTCTGCGGCAGGGCAGGGAGGGGGAAGAGTAGTCAGT
CGCTCGCTCACTCGCTCGCTCGCACAGACACTGCTGCAGTGACACTCGGC
CCTCCAGTGTCGCGGAGACGCAAGAGCAGCGCGCAGCACCTGTCCGCCCG
GAGCGAGCCCGGCCCGCGGCCGTAGAAAAGGAGGGACCGCCGAGGTGCGC
GTCAGTACTGCTCAGCCCGGCAGGGACGCGGGAGGATGTGGACTGGGTGG
AC
Blimp 1 promoter 2 kb (SEQ ID NO: 39):
GTGGTGCTGACTCAGCATCGGTTAATAAACCCTCTGCAGGAGGCTGGATT
TCTTTTGTTTAATTATCACTTGGACCTTTCTGAGAACTCTTAAGAATTGT
TCATTCGGGTTTTTTTGTTTTGTTTTGGTTTGGTTTTTTTGGGTTTTTTT
TTTTTTTTTTTTTTTGGTTTTTGGAGACAGGGTTTCTCTGTATATAGCCC
TGGCACAAGAGCAAGCTAACAGCCTGTTTCTTCTTGGTGCTAGCGCCCCC
TCTGGCAGAAAATGAAATAACAGGTGGACCTACAACCCCCCCCCCCCCCC
CCAGTGTATTCTACTCTTGTCCCCGGTATAAATTTGATTGTTCCGAACTA
CATAAATTGTAGAAGGATTTTTTAGATGCACATATCATTTTCTGTGATAC
CTTCCACACACCCCTCCCCCCCAAAAAAATTTTTCTGGGAAAGTTTCTTG
AAAGGAAAACAGAAGAACAAGCCTGTCTTTATGATTGAGTTGGGCTTTTG
TTTTGCTGTGTTTCATTTCTTCCTGTAAACAAATACTCAAATGTCCACTT
CATTGTATGACTAAGTTGGTATCATTAGGTTGGGTCTGGGTGTGTGAATG
TGGGTGTGGATCTGGATGTGGGTGGGTGTGTATGCCCCGTGTGTTTAGAA
TACTAGAAAAGATACCACATCGTAAACTTTTGGGAGAGATGATTTTTAAA
AATGGGGGTGGGGGTGAGGGGAACCTGCGATGAGGCAAGCAAGATAAGGG
GAAGACTTGAGTTTCTGTGATCTAAAAAGTCGCTGTGATGGGATGCTGGC
TATAAATGGGCCCTTAGCAGCATTGTTTCTGTGAATTGGAGGATCCCTGC
TGAAGGCAAAAGACCATTGAAGGAAGTACCGCATCTGGTTTGTTTTGTAA
TGAGAAGCAGGAATGCAAGGTCCACGCTCTTAATAATAAACAAACAGGAC
ATTGTATGCCATCATCACAGGATGTCCTTCCTTCTCCAGAAGACAGACTG
GGGCTGAAGGAAAAGCCGGCCAGGCTCAGAACGAGCCCCACTAATTACTG
CCTCCAACAGCTTTCCACTCACTGCCCCCAGCCCAACATCCCCTTTTTAA
CTGGGAAGCATTCCTACTCTCCATTGTACGCACACGCTCGGAAGCCTGGC
TGTGGGTTTGGGCATGAGAGGCAGGGACAACAAAACCAGTATATATGATT
ATAACTTTTTCCTGTTTCCCTATTTCCAAATGGTCGAAAGGAGGAAGTTA
GGTCTACCTAAGCTGAATGTATTCAGTTAGCAGGAGAAATGAAATCCTAT
ACGTTTAATACTAGAGGAGAACCGCCTTAGAATATTTATTTCATTGGCAA
TGACTCCAGGACTACACAGCGAAATTGTATTGCATGTGCTGCCAAAATAC
TTTAGCTCTTTCCTTCGAAGTACGTCGGATCCTGTAATTGAGACACCGAG
TTTAGGTGACTAGGGTTTTCTTTTGAGGAGGAGTCCCCCACCCCGCCCCG
CTCTGCCGCGACAGGAAGCTAGCGATCCGGAGGACTTAGAATACAATCGT
AGTGTGGGTAAACATGGAGGGCAAGCGCCTGCAAAGGGAAGTAAGAAGAT
TCCCAGTCCTTGTTGAAATCCATTTGCAAACAGAGGAAGCTGCCGCGGGT
CGCAGTCGGTGGGGGGAAGCCCTGAACCCCACGCTGCACGGCTGGGCTGG
CCAGGTGCGGCCACGCCCCCATCGCGGCGGCTGGTAGGAGTGAATCAGAC
CGTCAGTATTGGTAAAGAAGTCTGCGGCAGGGCAGGGAGGGGGAAGAGTA
GTCAGTCGCTCGCTCACTCGCTCGCTCGCACAGACACTGCTGCAGTGACA
CTCGGCCCTCCAGTGTCGCGGAGACGCAAGAGCAGCGCGCAGCACCTGTC
CGCCCGGAGCGAGCCCGGCCCGCGGCCGTAGAAAAGGAGGGACCGCCGAG
GTGCGCGTCAGTACTGCTCAGCCCGGCAGGGACGCGGGAGGATGTGGACT
GGGTGGAC

In some embodiments, the genome of a non-human animal as described herein further comprises one or more human immunoglobulin heavy and/or light chain genes (see, e.g., U.S. Pat. No. 8,502,018; U.S. Pat. No. 8,642,835; U.S. Pat. No. 8,697,940; U.S. Pat. No. 8,791,323; and U.S. Patent Application Publication No. 2013-0096287 A1; incorporated herein by reference). Alternatively, a disrupted or mutant Kynu gene can be introduced into an embryonic stem cell of a different modified strain such as, e.g., a VELOCIMMUNE® strain (see, e.g., U.S. Pat. No. 8,502,018 or U.S. Pat. No. 8,642,835; incorporated herein by reference). In some embodiments, a disrupted or mutant Kynu gene can be introduced into an embryonic stem cell of a modified strain as described in U.S. Pat. Nos. 8,697,940 and 8,642,835; incorporated herein by reference.

A transgenic founder non-human animal can be identified based upon the presence of a reporter gene (or absence of Kynu) in its genome and/or expression of a reporter in tissues or cells of the non-human animal (or lack of expression of Kynu), or the presence of one or more point mutations in a Kynu coding sequence (e.g., an exon) and/or a deletion of a non-coding Kynu sequence (e.g., an intron) in its genome and/or expression of a variant Kynu polypeptide in tissues or cells of the non-human animal (or lack of expression of wild-type Kynu polypeptide). A transgenic founder non-human animal can then be used to breed additional non-human animals carrying the reporter gene or mutant Kynu gene thereby creating a series of non-human animals each carrying one or more copies of a disrupted or mutant Kynu gene as described herein.

Transgenic non-human animals may also be produced to contain selected systems that allow for regulated or directed expression of a transgene or polynucleotide molecule (e.g., an insert nucleic acid). Exemplary systems include the Cre/loxP recombinase system of bacteriophage P1 (see, e.g., Lakso, M. et al., 1992, Proc. Natl. Acad. Sci. USA 89:6232-6236) and the FLP/Frt recombinase system of S. cerevisiae (O′Gorman, S. et al, 1991, Science 251:1351-1355). Such animals can be provided through the construction of “double” transgenic animals, e.g., by mating two transgenic animals, one containing a transgene encoding a selected polypeptide (e.g., a reporter or variant Kynu polypeptide) and the other containing a transgene encoding a recombinase (e.g., a Cre recombinase).

The non-human animals as described herein may be prepared as described above, or using methods known in the art, to comprise additional human or humanized genes, oftentimes depending on the intended use of the non-human animal. Genetic material of such additional human or humanized genes may be introduced through the further alteration of the genome of cells (e.g., embryonic stem cells) having genetic modifications as described herein or through breeding techniques known in the art with other genetically modified strains as desired. In some embodiments, non-human animals as described herein are prepared to further comprise transgenic human immunoglobulin heavy and light chain genes (see, e.g., Murphy, A. J. et al., 2014, Proc. Natl. Acad. Sci. U.S.A. 111(14):5153-5158; U.S. Pat. Nos. 8,502,018, 8,642,835, 8,697,940 and 8,791,323; and U.S. Patent Application Publication No. 2013-0096287 A1; all of which are incorporated herein by reference in their entirety).

In some embodiments, non-human animals as described herein may be prepared by introducing a targeting vector as described herein into a cell from a modified strain. To give but one example, a targeting vector as described above may be introduced into a VELOCIMMUNE® mouse cell (e.g., an embryonic stem cell). VELOCIMMUNE® mice express antibodies that have fully human variable regions and mouse constant regions. In some embodiments, non-human animals as described herein are prepared to further comprise human immunoglobulin genes (variable and/or constant region genes). In some embodiments, non-human animals as described herein comprise a disrupted or mutant Kynu gene as described herein and genetic material from a heterologous species (e.g., humans), wherein the genetic material encodes, in whole or in part, one or more human heavy and/or light chain variable regions.

For example, as described herein, non-human animals comprising a disrupted or mutant Kynu gene as described herein may further comprise (e.g., via cross-breeding or multiple gene targeting strategies) one or more modifications as described in Murphy, A. J. et al., 2014, Proc. Natl. Acad. Sci. U.S.A. 111(14):5153-5158; U.S. Pat. Nos. 8,502,018, 8,642,835, 8,697,940 and 8,791,323; U.S. Patent Application Publication No. 2013-0096287 A1; all of which are incorporated herein by reference in their entirety. In some embodiments, a rodent comprising a disrupted or mutant Kynu gene as described herein is crossed to a rodent comprising a humanized immunoglobulin heavy and/or light chain variable region locus (see, e.g., U.S. Pat. Nos. 8,502,018 or 8,642,835; incorporated herein by reference).

Although embodiments employing a disruption or mutation in a Kynu gene in a mouse are extensively discussed herein, other non-human animals that comprise such modifications (or alterations) in a Kynu gene locus are also provided. In some embodiments, such non-human animals comprise a disruption in a Kynu gene (e.g., a mouse with a deletion of a portion of a Kynu coding sequence) characterized by insertion of a reporter operably linked to an endogenous Kynu promoter or a mutation in a Kynu gene (e.g., a mouse with one or more point mutations in one or more Kynu exons) characterized by insertion of a mutant Kynu exon or exons (e.g., an exon three that contains one or more point mutations) operably linked to an endogenous Kynu promoter. Such non-human animals include any of those which can be genetically modified to disrupt or mutate a coding sequence of a Kynu gene as disclosed herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. For example, for those non-human animals for which suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing somatic cell nuclear transfer (SCNT) to transfer the genetically modified genome to a suitable cell, e.g., an enucleated oocyte, and gestating the modified cell (e.g., the modified oocyte) in a non-human animal under suitable conditions to form an embryo.

Briefly, methods for nuclear transfer include steps of: (1) enucleating an oocyte; (2) isolating a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods oocytes are generally retrieved from deceased animals, although they may be isolated also from either oviducts and/or ovaries of live animals. Oocytes may be matured in a variety of medium known to persons of skill in the art prior to enucleation. Enucleation of the oocyte can be performed in a variety of ways known to persons of skill in the art. Insertion of a donor cell or nucleus into an enucleated oocyte to form a reconstituted cell is typically achieved by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell is typically activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, are typically cultured in medium known to persons of skill in the art and then transferred to the womb of an animal. See, e.g., U.S. Pat. No. 7,612,250; U.S. Patent Application Publication Nos. 2004-0177390 A1 and 2008-0092249 A1; and International Patent Application Publication Nos. WO 1999/005266 A2 and WO 2008/017234 A1; each of which is incorporated herein by reference.

Methods for modifying a non-human animal genome (e.g., a pig, cow, rodent, chicken, etc. genome) include, e.g., employing a zinc finger nuclease (ZFN), a transcription activator-like effector nuclease (TALEN), or a Cas protein (i.e., a CRISPR/Cas system) to modify a genome to include a disrupted or mutant Kynu gene as described herein.

In some embodiments, a non-human animal of the present invention is a mammal. In some embodiments, a non-human animal as described herein is a small mammal, e.g., of the superfamily Dipodoidea or Muroidea. In some embodiments, a non-human animal as described herein is a rodent. In some embodiments, a rodent as described herein is selected from a mouse, a rat, and a hamster. In some embodiments, a rodent as described herein is selected from the superfamily Muroidea. In some embodiments, a genetically modified animal as described herein is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rates, bamboo rats, and zokors). In some certain embodiments, a rodent as described herein is selected from a true mouse or rat (family Muridae), a gerbil, a spiny mouse, and a crested rat. In some certain embodiments, a mouse as described herein is from a member of the family Muridae. In some embodiment, a non-human animal as described herein is a rodent. In some certain embodiments, a rodent as described herein is selected from a mouse and a rat. In some embodiments, a non-human animal as described herein is a mouse.

In some embodiments, a non-human animal as described herein is a rodent that is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some certain embodiments, a mouse as described herein is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129/SvJae, 129S6 (129/SvEvTac), 129S7, 129S8, 129T1, 129T2 (see, e.g., Festing et al., 1999, Mammalian Genome 10:836; Auerbach, W. et al., 2000, Biotechniques 29(5):1024-1028, 1030, 1032). In some certain embodiments, a genetically modified mouse as described herein is a mix of an aforementioned 129 strain and an aforementioned C57BL/6 strain. In some certain embodiments, a mouse as described herein is a mix of aforementioned 129 strains, or a mix of aforementioned BL/6 strains. In some certain embodiments, a 129 strain of the mix as described herein is a 129S6 (129/SvEvTac) strain. In some embodiments, a mouse as described herein is a BALB strain, e.g., BALB/c strain. In some embodiments, a mouse as described herein is a mix of a BALB strain and another aforementioned strain.

In some embodiments, a non-human animal as described herein is a rat. In some certain embodiments, a rat as described herein is selected from a Wistar rat, an LEA strain, a Sprague Dawley strain, a Fischer strain, F344, F6, and Dark Agouti. In some certain embodiments, a rat strain as described herein is a mix of two or more strains selected from the group consisting of Wistar, LEA, Sprague Dawley, Fischer, F344, F6, and Dark Agouti.

A rat pluripotent and/or totipotent cell can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rat pluripotent and/or totipotent cells can also be obtained from a strain derived from a mix of two or more strains recited above. For example, a rat pluripotent and/or totipotent cell can be from a DA strain or an ACI strain. An ACI rat strain is characterized as having black agouti, with white belly and feet and an RT1^{av1} haplotype. Such strains are available from a variety of sources including Harlan Laboratories. An example of a rat ES cell line from an ACI rat is an ACI.G1 rat ES cell. A Dark Agouti (DA) rat strain is characterized as having an agouti coat and an RT1^{avI} haplotype. Such rats are available from a variety of sources including Charles River and Harlan Laboratories. Examples of a rat ES cell line from a DA rat are the DA.2B rat ES cell line and the DA.2C rat ES cell line. In some cases, rat pluripotent and/or totipotent cells are from an inbred rat strain. See, e.g., U.S. Patent Application Publication No. 2014-0235933 A1, incorporated herein by reference.

Non-human animals are provided that comprise a disruption in a Kynu gene. In some embodiments, a disruption in a Kynu gene results in a loss-of-function. In particular, loss-of-function mutations include mutations that result in a decrease or lack of expression of Kynu and/or a decrease or lack of activity/function of Kynu. In some embodiments, loss-of-function mutations result in one or more phenotypes as compared to wild-type non-human animals. Expression of Kynu may be measured directly, e.g., by assaying the level of Kynu in a cell or tissue of a non-human animal as described herein.

Typically, expression level and/or activity of Kynu is decreased if the expression and/or activity level of Kynu is statistically lower (p<0.05) than the level of Kynu in an appropriate control cell or non-human animal that does not comprises the same disruption (e.g., deletion). In some embodiments, concentration and/or activity of Kynu is decreased by at least 1%, 5%, 10%, 20%, 30%, 40%, 50%, 60%, 70%, 80%, 90%, 95%, 99% or more relative to a control cell or non-human animal which lacks the same disruption (e.g., deletion).

In other embodiments, cells or organisms having a disruption in a Kynu gene that reduces the expression level and/or activity of Kynu are selected using methods that include, but not limited to, Southern blot analysis, DNA sequencing, PCR analysis, or phenotypic analysis. Such cells or non-human animals are then employed in various methods and compositions described herein.

In some embodiments, an endogenous Kynu gene is not deleted (i.e., intact). In some embodiments, an endogenous Kynu gene is altered, disrupted, deleted or replaced with a heterologous sequence (e.g., a reporter gene encoding sequence). In some embodiments, all or substantially all of an endogenous Kynu gene is replaced with an insert nucleic acid; in some certain embodiments, replacement includes replacement of a portion of the coding sequence of an endogenous Kynu gene with a reporter gene (e.g., lacZ) so that the reporter gene is in operable linkage with a Kynu promoter (e.g., an endogenous Kynu promoter). In some embodiments, a portion of a reporter gene (e.g., a function fragment thereof) is inserted into an endogenous non-human Kynu gene. In some embodiments, a reporter gene is a lacZ gene. In some embodiments, a reporter gene is inserted into one of the two copies of an endogenous Kynu gene, giving rise to a non-human animal that is heterozygous with respect to the reporter gene. In some embodiments, a non-human animal is provided that is homozygous for a reporter gene.

Non-human animals are provided that comprise a mutation(s) in a Kynu gene. In some embodiments, a mutation in a Kynu gene results in the expression of a variant Kynu polypeptide (e.g., a Kynu polypeptide that includes one or more amino acid substitutions as compared to a wild-type Kynu polypeptide). Expression of variant Kynu may be measured directly, e.g., by assaying the level of variant Kynu in a cell or tissue of a non-human animal as described herein.

In other embodiments, cells or organisms having a mutation(s) in a Kynu gene are selected using methods that include, but not limited to, Southern blot analysis, DNA sequencing, PCR analysis, or phenotypic analysis. Such cells or non-human animals are then employed in various methods and compositions described herein.

In some embodiments, an endogenous Kynu gene is altered or replaced with a mutant Kynu sequence (e.g., a mutant Kynu-encoding sequence, in whole or in part). In some embodiments, all or substantially all of an endogenous Kynu gene is replaced with an insert nucleic acid; in some certain embodiments, replacement includes replacement of an endogenous Kynu exon three with a mutant Kynu exon three so that the mutant Kynu exon three is in operable linkage with a Kynu promoter (e.g., an endogenous Kynu promoter) and other endogenous Kynu exons. In some embodiments, a mutant Kynu exon three is inserted into an endogenous Kynu gene, which mutant Kynu exon three contains one or more point mutations; in some certain embodiments, five point mutations. In some embodiments, a mutant Kynu exon three is inserted into one of the two copies of an endogenous Kynu gene, giving rise to a non-human animal that is heterozygous with respect to the mutant Kynu exon three. In some embodiments, a non-human animal is provided that is homozygous for a mutant Kynu exon three. In some embodiments, non-human animals that comprise a mutant endogenous Kynu gene further comprise a Kynu intron three that includes a deletion (e.g., about 60 bp) and/or a site-specific recombinase recognition site (e.g., loxP).

### Methods Employing Non-Human Animals Having a Mutant Kynu Gene

Non-human animals described herein provide improved animal models for HIV infection and/or transmission. In particular, non-human animals as described herein provide improved animal models that translate to HIV-related diseases, disorders and conditions characterized by, for example, progressive immune system failure, secondary opportunistic infections, loss of cell-mediated immunity and cancer.

For example, a disruption in a Kynu gene as described herein may result in various symptoms (or phenotypes) in non-human animals provided herein. In some embodiments, disruption of a Kynu gene results in non-human animals that are grossly normal at birth, but that develop one or more symptoms upon aging, e.g., after about 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, etc. In some embodiments, disruption of a Kynu gene results in non-human animals having abnormal functions of one or more cell types. In some embodiments, disruption of a Kynu gene results in non-human animals demonstrating one or more symptoms (or phenotypes) associated with hypertension and/or renal disease. Such symptoms (or phenotypes) may include, for example, high blood pressure (i.e., increased resistance to blood flow), insulin resistance, decreased arterial compliance, enlarged ventricle(s) and hypertensive retinopathy. In some embodiments, non-human animals described herein provide improved in vivo systems for identifying and developing candidate therapeutics for the treatment of stroke. Thus, in at least some embodiments, non-human animals described herein provide improved animal models for hypertension and/or renal disease and can be used for the development and/or identification of therapeutic agents for the treatment and/or prevention of hypertensive diseases, disorders or conditions.

Non-human animals as described herein provide an improved in vivo system and source of biological materials (e.g., cells) that lack expression of Kynu or that express variant Kynu polypeptides that are useful for a variety of assays. In various embodiments, non-human animals described herein are used to develop therapeutics that treat, prevent and/or inhibit one or more symptoms associated with a lack of Kynu expression and/or activity. In various embodiments, non-human animals described herein are used to develop therapeutics that treat, prevent and/or inhibit one or more symptoms associated with expression of variant Kynu polypeptides. Due to the expression of variant Kynu polypeptides, non-human animals described herein are useful for use in various assays to determine the functional consequences on the kynurenine pathway. In some embodiments, non-human animals described herein provide an animal model for screening molecules that act on one or more enzymes (or products of) in the kynurenine pathway.

Other phenotypes may be present in non-human animals described herein. For example, in some embodiments, a disruption or mutation of a Kynu gene as described herein results in the capacity of a non-human animal described herein to mount an immune response (e.g., an antibody response) against HIV. Such an immune response may be characterized by the presence of neutralizing antibodies to one or more epitopes present on HIV in non-human animals described herein. Thus, in at least some embodiments, non-human animals described herein provide improved animal models for HIV infection and/or transmission and can be used for the development and/or identification of therapeutic agents for the treatment, prevention and/or inhibition of HIV-related diseases, disorders or conditions.

Non-human animals described herein also provide an in vivo system for identifying a therapeutic agent for treating, preventing and/or inhibiting progressive failure of the immune system resulting from prolonged HIV infection. In some embodiments, an effect of a therapeutic agent is determined in vivo, by administering said therapeutic agent to a non-human animal whose genome comprises a Kynu gene as described herein.

Non-human animals described herein also provide improved animal models for dysfunctional cell-mediated immunity. In particular, non-human animals as described herein provide improved animal models that translate to conditions characterized by progressive decline of immune cells (e.g., helper T cells). In addition, non-human animals as described herein provide improved animal models that translate to conditions related to acquired immunodeficiency syndrome (AIDS).

Non-human animals may be administered a therapeutic agent to be tested by any convenient route, for example, by intravenous or intraperitoneal injection. Such animals may be included in an immunological study, so as to determine the effect of the therapeutic agent on the immune system (e.g., effect on T cells) of the non-human animals as compared to appropriate control non-human animals that did not receive the therapeutic agent. A biopsy or anatomical evaluation of animal tissue (e.g., lymphoid tissue) may also be performed, and/or a sample of blood may be collected.

In some embodiments, non-human animals described herein provide an in vivo system for generating antibodies that bind an HIV envelope. In some embodiments, prevention of HIV infection and/or transmission by an antibody is determined in vivo, by administering said antibody to a non-human animal whose genome comprises a Kynu gene as described herein.

In various embodiments, non-human animals described herein are used to identify, screen and/or develop candidate therapeutics (e.g., antibodies) that bind HIV (e.g., an HIV envelope) and, in some embodiments, block HIV infection and/or transmission. In various embodiments, non-human animals described herein are used to determine the binding profile of candidate therapeutics (e.g., antibodies) that bind HIV; in some certain embodiments, an HIV envelope. In some embodiments, non-human animals described herein are used to determine the epitope or epitopes of one or more candidate therapeutic antibodies that bind HIV.

In various embodiments, non-human animals described herein are used to determine the pharmacokinetic profiles of a drug targeting HIV. In various embodiments, one or more non-human animals described herein and one or more control or reference non-human animals are each exposed to one or more candidate drugs targeting HIV at various doses (e.g., 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/mg, 7.5 mg/kg, 10 mg/kg, 15 mg/kg, 20 mg/kg, 25 mg/kg, 30 mg/kg, 40 mg/kg, or 50 mg/kg or more). Candidate therapeutic drugs targeting HIV may be dosed via any desired route of administration including parenteral and non-parenteral routes of administration. Parenteral routes include, e.g., intravenous, intraarterial, intraportal, intramuscular, subcutaneous, intraperitoneal, intraspinal, intrathecal, intracerebroventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection. Blood is isolated from non-human animals at various time points (e.g., 0 hr, 6 hr, 1 day, 2 days, 3 days, 4 days, 5 days, 6 days, 7 days, 8 days, 9 days, 10 days, 11 days, or up to 30 or more days). Various assays may be performed to determine the pharmacokinetic profiles of administered drugs targeting HIV using samples obtained from non-human animals described herein including, but not limited to, total IgG, anti-drug response, agglutination, etc.

In various embodiments, non-human animals as described herein are used to measure the therapeutic effect of blocking, modulating, and/or inhibiting HIV activity (e.g., infection, replication, spread, etc.) and the effect on gene expression as a result of cellular changes in the immune system. In various embodiments, a non-human animal as described herein or cells isolated therefrom are exposed to a drug targeting HIV and, after a subsequent period of time, analyzed for effects on HIV-dependent processes (or interactions), for example, membrane fusion with T cells, viral replication, or genetic variability among viral isolates.

Cells from non-human animals as described herein can be isolated and used on an ad hoc basis, or can be maintained in culture for many generations. In various embodiments, cells from a non-human animal described herein are immortalized (e.g., via use of a virus, cell fusion, etc.) and maintained in culture indefinitely (e.g., in serial cultures).

In various embodiments, B cells of non-human animals described herein are used in the production of antibodies that bind HIV. For example, B cells may be isolated from non-human animals described herein and used directly or immortalized for the generation of hybridomas. Such non-human animals may be immunized with HIV or an HIV-associated antigen (e.g., peptide comprising a sequence that appears in an HIV envelope polypeptide) prior to isolation of B cells from the non-human animals. Alternatively, B cells may be isolated from non-human animals described herein prior to employing an immunization regimen. B cells and/or hybridomas can be screened for binding to various HIV-related antigens and characterized by affinity and/or epitope. Antibodies may be cloned and sequenced from such cells and used to generate candidate therapeutics (or candidate therapeutic libraries) that can be used in further assays to determine various properties of the antibodies as desired.

Non-human animals described herein provide an in vivo system for the analysis and testing of a drug or vaccine. In various embodiments, a candidate drug or vaccine may be delivered to one or more non-human animals described herein, followed by monitoring of the non-human animals to determine one or more of the immune response to the drug or vaccine, the safety profile of the drug or vaccine, or the effect on a disease or condition and/or one or more symptoms of a disease or condition. Exemplary methods used to determine the safety profile include measurements of toxicity, optimal dose concentration, efficacy of the drug or vaccine, and possible risk factors. Such drugs or vaccines may be improved and/or developed in such non-human animals. In some embodiments, non-human animals described herein are used for the analysis, testing and/or development of an HIV vaccine.

Vaccine efficacy may be determined in a number of ways. Briefly, non-human animals described herein are vaccinated using methods known in the art and then challenged with a vaccine, or a vaccine is administered to already-infected non-human animals. The response of a non-human animal(s) to a vaccine may be measured by monitoring of, and/or performing one or more assays on, the non-human animal(s) (or cells isolated therefrom) to determine the efficacy of the vaccine. The response of a non-human animal(s) to the vaccine is then compared with control animals, using one or more measures known in the art and/or described herein.

Vaccine efficacy may further be determined by viral neutralization assays. Briefly, non-human animals described herein are immunized and serum is collected on various days post-immunization. Serial dilutions of serum are pre-incubated with a virus during which time antibodies in the serum that are specific for the virus will bind to it. The virus/serum mixture is then added to permissive cells to determine infectivity by a plaque assay or microneutralization assay. If antibodies in the serum neutralize the virus, there are fewer plaques or lower relative luciferase units compared to a control group.

Non-human animals described herein provide an in vivo system for assessing the pharmacokinetic properties and/or efficacy of a drug. In various embodiments, a drug may be delivered or administered to one or more non-human animals described herein, followed by monitoring of, or performing one or more assays on, the non-human animals (or cells isolated therefrom) to determine the effect of the drug on the non-human animal. Pharmacokinetic properties include, but are not limited to, how a non-human animal processes the drug into various metabolites (or detection of the presence or absence of one or more drug metabolites, including, but not limited to, toxic metabolites), drug half-life, circulating levels of drug after administration (e.g., serum concentration of drug), anti-drug response (e.g., anti-drug antibodies), drug absorption and distribution, route of administration, routes of excretion and/or clearance of the drug. In some embodiments, pharmacokinetic and pharmacodynamic properties of drugs are monitored in or through the use of non-human animals described herein.

In some embodiments, performing an assay includes determining the effect on the phenotype and/or genotype of the non-human animal to which the drug is administered. In some embodiments, performing an assay includes determining lot-to-lot variability for a drug. In some embodiments, performing an assay includes determining the differences between the effects of a drug administered to a non-human animal described herein and a reference non-human animal. In various embodiments, reference non-human animals may have a modification described herein, a modification that is different than described herein or no modification (i.e., a wild-type non-human animal).

Exemplary parameters that may be measured in non-human animals (or in and/or using cells isolated therefrom) for assessing the pharmacokinetic properties of a drug include, but are not limited to, agglutination, autophagy, cell division, cell death, complement-mediated hemolysis, DNA integrity, drug-specific antibody titer, drug metabolism, gene expression arrays, metabolic activity, mitochondrial activity, oxidative stress, phagocytosis, protein biosynthesis, protein degradation, protein secretion, stress response, target tissue drug concentration, non-target tissue drug concentration, transcriptional activity, and the like. In various embodiments, non-human animals described herein are used to determine a pharmaceutically effective dose of a drug.

### Kits

The present invention further provides a pack or kit comprising one or more containers filled with at least one non-human animal, non-human cell, DNA fragment, and/or targeting vector as described herein. Kits may be used in any applicable method (e.g., a research method). Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both, or a contract that governs the transfer of materials and/or biological products (e.g., a non-human animal or a non-human cell as described herein) between two or more entities.

Other features of the invention will become apparent in the course of the following descriptions of exemplary embodiments, which are given for illustration and are not intended to be limiting thereof.

### EXAMPLES

The following examples are provided so as to describe to those of ordinary skill in the art how to make and use methods and compositions of the invention, and are not intended to limit the scope of what the inventors regard as their invention. Unless indicated otherwise, temperature is indicated in Celsius, and pressure is at or near atmospheric.

### Example 1. Generation of a Disruption in a Rodent Kynureninase (Kynu) Gene

This example illustrates the construction of a targeting vector for creating a disruption in a kynureninase (Kynu) locus of a rodent. In particular, this example specifically describes the deletion of a 5′ portion of the coding sequence (i.e., beginning 3′ of ATG codon in exon two to five base pairs before the 3′ end of exon six resulting in a 39,343 bp deletion) of a mouse Kynu gene using a lacZ reporter construct placed in operable linkage with a mouse Kynu promoter (i.e., in frame with ATG codon of exon 2). The Kynu-lacZ-SDC targeting vector for creating a disruption in an endogenous mouse Kynu locus was constructed as previously described (see, e.g., U.S. Pat. No. 6,586,251; Valenzuela et al., 2003, Nature Biotech. 21(6):652-659; and Adams, N.C. and N.W. Gale, in Mammalian and Avian Transgenesis—New Approaches, ed. Lois, S.P.a.C., Springer Verlag, Berlin Heidelberg, 2006). An exemplary targeting vector (or DNA construct) is set forth in FIGS. 2A-2C.

Briefly, the Kynu-lacZ-SDC targeting vector was generated using mouse bacterial artificial chromosome (BAC) clone RP23-391p24 (Invitrogen) and a self-deleting neomycin selection cassette (LacZ-pA-ICeuI-loxP-mPrm1-Crei-SV40 pA-hUbl-em7-Neo-PGKpA-loxP) as previously described (see, U.S. Pat. Nos. 8,697,851, 8,518,392 and 8,354,389; all of which are incorporated herein by reference). The Kynu-lacZ-SDC targeting vector included a Cre recombinase-encoding sequence that is operably linked to mouse protamine 1 promoter that is developmentally regulated such that the recombinase is expressed in undifferentiated cells. Upon homologous recombination, a deletion including nucleotides 3′ of the ATG codon in exon two to five base pairs before the 3′ end of exon 6 (39,343 bp) of an endogenous murine Kynu gene is replaced by the sequence contained in the targeting vector (˜8,430 bp). The drug selection cassette is removed in a development-dependent manner, i.e., progeny derived from mice whose germ line cells containing a disrupted Kynu gene described above will shed the selectable marker from differentiated cells during development (see U.S. Pat. Nos. 8,697,851, 8,518,392 and 8,354,389, all of which are incorporated herein by reference).

Construction of the Kynu-lacZ-SDC targeting vector was confirmed by polymerase chain reaction and sequence analysis, and then introduced into mouse embryonic stem (ES) cells followed by culturing in selection medium containing G418. The mouse E S cells used for electroporation had a genome that included a plurality of human V_{H}, D_{H} and J_{H} segments operably linked with rodent immunoglobulin heavy chain constant regions (e.g., IgM, IgD, IgG, etc.), a plurality of human Vκ and Jκ segments operably linked with a rodent immunoglobulin κ light chain constant region, and an inserted sequence encoding one or more murine Adam6 genes (see, e.g., U.S. Pat. Nos. 8,642,835 and 8,697,940; both of which are incorporated herein by reference). Drug-resistant clones were picked 10 days after electroporation and screened by TAQMAN™ and karyotyping for correct targeting as previously described (Valenzuela et al., supra; Frendewey, D. et al., 2010, Methods Enzymol. 476:295-307) using primer/probe sets that detected deletion proper deletion of ˜39.4 kb of an endogenous Kynu gene (Table 1 and FIG. 2B).

The nucleotide sequence across the upstream junction point included the following, which indicates endogenous mouse Kynu intron 1 sequence and a mouse Kynu ATG codon (contained within the parentheses with the ATG codon in uppercase font) contiguous with lacZ coding sequence (italicized uppercase font with a KpnI site underlined): (ttttacttcc ttcttagata acagttt ATG) GGTACC GATTTAAATG ATCCAGTGGT CCTGCAGAGG AGAGATTGG (SEQ ID NO:15).

The nucleotide sequence across the downstream junction point included the following, which indicates cassette sequence (lowercase font with an NheI site underlined) contiguous with the last five base pairs of exon 6 and 34 bp of intron 6 of a mouse Kynu gene (contained within the parentheses with coding sequence in uppercase font and noncoding sequence in lowercase font): ataacttcgt ataatgtatg ctatacgaag ttat gctagc (GAGAG gtatctgtga aagaaagaaa tgctcattag actt) (SEQ ID NO:16).

The nucleotide sequence across the upstream junction point after recombinase-mediated excision of the selection cassette (3,470 bp remain 3′ of ATG codon) included the following, which indicates endogenous mouse Kynu intron 1 sequence and a mouse Kynu ATG codon (contained within the parentheses with the ATG codon in uppercase font) contiguous with lacZ coding sequence (italicized uppercase font with a KpnI site underlined): (ttttacttcc ttcttagata acagttt ATG) GGTACC GATTTAAATG ATCCAGTGGT CCTGCAGAGG AGAGATTGG (SEQ ID NO:15).

The nucleotide sequence across the downstream junction point after recombinase-mediated excision of the selection cassette (3,470 bp remain 3′ of ATG codon) included the following, which indicates remaining lacZ sequence (italicized uppercase font with ICeu-I, loxP and NheI sites underlined and in lowercase font) contiguous with the last five base pairs of exon 6 and 34 bp of intron 6 of a mouse Kynu gene (contained within the parentheses with coding sequence in

(SEQ ID NO: 17)
CTCATCAATG TATCTTATCA TGTCTGGATC CCC cggctagagt
ttaaacacta gaactagtgg atccccgggc taactataac
ggtcctaagg tagcga ctcgac ataacttcgt ataatgtatg
ctatacgaag ttat gctagc (GAGAG gtatctgtga
aagaaagaaa tgctcatta).

After four separate attempts with the Kynu-lacZ-SDC targeting vector, no positive ES clones for disruption of a mouse Kynu gene as described above were confirmed. In another experiment, disruption of a mouse Kynu gene as described above was accomplished using a hybrid ES cell line, F1H4 (50% 129/S6/SvEv/Tac, 50% C57BL/6NTac; Auerbach, W. et al. (2000) Biotechniques 29(5):1024-8, 1030, 1032).

TABLE 1

| Name | Primer | Sequence (5′-3′) |
|---|---|---|
| 4249mTU | Forward | TGCTACCCTACCAACCCATC |
| | | (SEQ ID NO: 18) |
| | Probe | CCTACCCGAGCCTCGTGTTCTTTACG |
| | | (SEQ ID NO: 19) |
| | Reverse | GACAGCGTAAACACCCTGAGAG |
| | | (SEQ ID NO: 20) |
| 4249mTD2 | Forward | ATTCTGCACTTCTGATCACCTTTA |
| | | (SEQ ID NO: 21) |
| | Probe | TCAACAAGTACCCTGATTCACATTAAGGA |
| | | (SEQ ID NO: 22) |
| | Reverse | GAATGGCTACCTCACAGACATC |
| | | (SEQ ID NO: 23) |

Taken together, this example demonstrates that elimination of a gene product by deletion of a coding sequence, in whole or in part, may not be feasible for some genetic loci. Further, this example demonstrates that, in some embodiments, other approaches to modify a genetic locus (or loci) so that shared epitopes present in an endogenous polypeptide(s) and a foreign entity (e.g., a virus) are not expressed, but otherwise encoding, producing or expressing a functional polypeptide, may be required.

### Example 2. Generation of a Mutation in a Rodent Kynureninase (Kynu) Gene

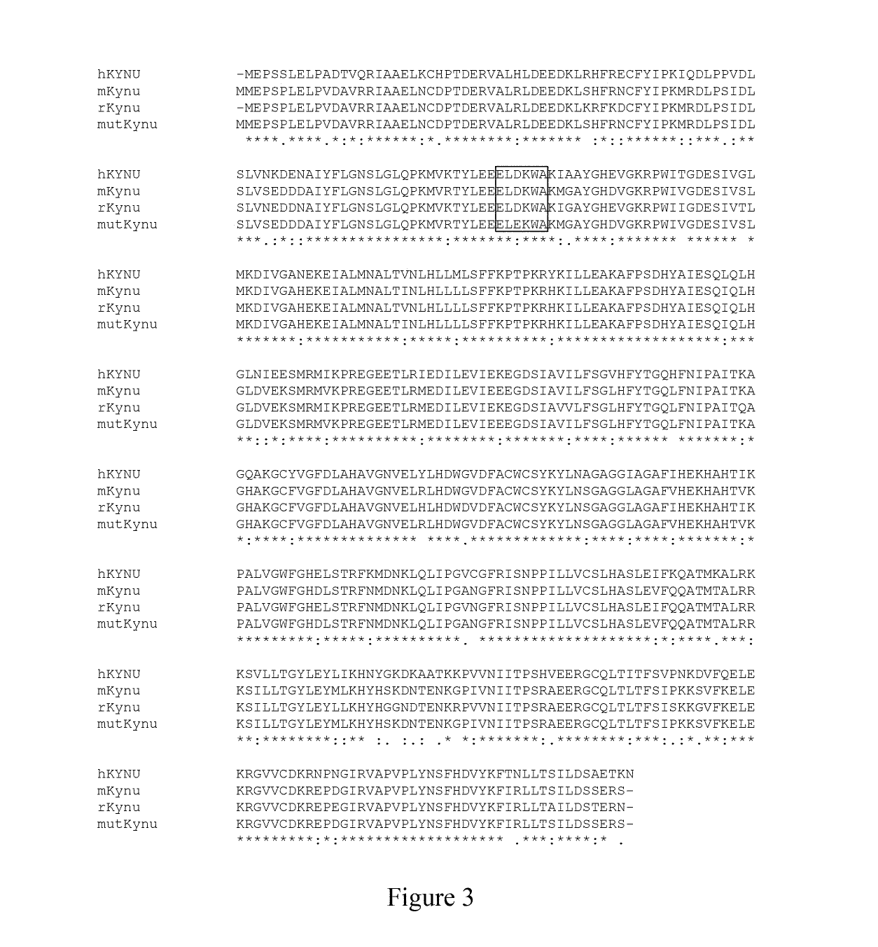

This example illustrates exemplary methods for creating one or more point mutations in an endogenous kynureninase (Kynu) locus in a non-human mammal such as a rodent (e.g., a mouse) that results in the elimination of a shared epitope present in a Kynu polypeptide and the MPER of HIV-1 gp41. Alignment of human, mouse, rat and mutant Kynu (as described below) amino acid sequences, with a shared epitope of HIV-1 gp41 and Kynu bound by monoclonal antibody 2F5 boxed, is set forth in FIG. 3. FIGS. 4A-4D show an exemplary targeting vector for creating point mutations in the genetic material encoding a rodent Kynu polypeptide that was constructed using VELOCIGENE® technology (see, e.g., U.S. Pat. No. 6,586,251 and Valenzuela et al., 2003, Nature Biotech. 21(6):652-659; all of which are incorporated herein by reference).

Briefly, mouse bacterial artificial chromosome (BAC) clone bMQ-280G7 (Adams, D. J. et al., 2005, Genomics 86:753-758) was modified to introduce a point mutation in exon three of an endogenous Kynu gene so that a Kynu polypeptide having a D93E amino acid substitution would be expressed (FIGS. 2 and 4B). Four additional synonymous point mutations were made in exon three and a ˜60 bp deletion in intron three (i.e., downstream, or 3′, of the D93E substitution) were introduced to facilitate screening of positive clones (FIG. 4B). Point mutations and the ˜60 bp deletion in intron three were introduced by de novo DNA synthesis using small flanking arms (i.e., 250 bp and 100 bp, respectively, 5′ and 3′ to the mutated region) identical in sequence to mouse sequence flanking the targeted region (synthesized by GeneScript, Piscataway, N.J.). The synthesized fragment (609 bp) was contained in a plasmid backbone and propagated in bacteria under selection with ampicillin. A hygromycin resistance gene was cloned into the synthetic fragment using restriction enzymes to create a donor plasmid for homologous recombination with the bMQ-280G7 BAC (FIG. 4C). The resulting modified bMQ-280G7 BAC clone was then electroporated into ES cells. The KynuD93E-SDC targeting vector included a Cre recombinase-encoding sequence that is operably linked to mouse protamine 1 promoter that is developmentally regulated such that the recombinase is expressed in undifferentiated cells (FIG. 4C; see also, U.S. Pat. Nos. 8,697,851, 8,518,392 and 8,354,389; all of which are incorporated herein by reference). Upon homologous recombination, the 20 bp synthetic mutated Kynu exon three is inserted in the place of the last 20 bp of exon three of an endogenous murine Kynu locus and about 60 bp of intron three of an endogenous murine Kynu locus is deleted by the sequence contained in the targeting vector (˜8,430 bp). The drug selection cassette is removed in a development-dependent manner, i.e., progeny derived from mice whose germ line cells containing a mutated Kynu gene described above will shed the selectable marker from differentiated cells during development (FIG. 4D; see also U.S. Pat. Nos. 8,697,851, 8,518,392 and 8,354,389, all of which are incorporated herein by reference). Endogenous DNA containing surrounding exons, introns and untranslated regions (UTRs) were unaltered by the mutagenesis and selection cassette. Sequence analysis of the targeting vector confirmed all exons, introns, splicing signals and the open reading frame of the mutant Kynu gene.

The modified bMQ-280G7 BAC clone described above was used to electroporate mouse embryonic stem (ES) cells to create modified ES cells comprising a mutant Kynu gene that encodes a Kynu polypeptide that contains a D93E substitution. The mouse ES cells used for electroporation had a genome that included a plurality of human V_{H}, D_{H} and J_{H} segments operably linked with rodent immunoglobulin heavy chain constant regions (e.g., IgM, IgD, IgG, etc.), a plurality of human Vκ and Jκ segments operably linked with rodent immunoglobulin κ light chain constant region, and an inserted sequence encoding one or more murine Adam6 genes (see, e.g., U.S. Pat. Nos. 8,642,835 and 8,697,940; both of which are incorporated herein by reference). Drug-resistant clones were picked 10 days after electroporation and screened by TAQMAN™ and karyotyping for correct targeting as previously described (Valenzuela et al., supra; Frendewey, D. et al., 2010, Methods Enzymol. 476:295-307) using primer/probe sets that detected proper introduction of the point mutations in exon three and deletion in intro three into an endogenous Kynu gene (Table 2 and FIG. 4C).

Screening clones using 4247mTD (Table 2) confirmed proper integration of the selection cassette as it was designed to amplify only the wild-type allele due to the location being within a small deletion created in the mutant (i.e., a 60 bp deletion in intron three). Screening clones with 4247mTU2_D93E (Table 2) confirmed proper integration of the mutant Kynu exon three as it was designed to amplify only the mutant exon three thereby detecting the presence of the engineered point mutations.

The nucleotide sequence across the upstream junction point included the following, which indicates endogenous mouse Kynu exon three sequence (uppercase font contained within the parentheses below with point mutations in bold and underlined font) contiguous with cassette sequence (lowercase font with a XhoI site underlined and a loxP site in bold font) at the insertion point: (TTCCTGGGAA ATTCCCTTGG CCTTCAACCG AAAATGGTTA GGACATACCT GGAGGAAGAG CTTGAAAAAT GGGCTAAGAT GTAAGTACCA AGTTAAAAGG TGTAACTCCA TCTGACAGAA GAATTCTGAA AATTACAAAA TGTGTCTGAT TTGGACAAGT TACACCCTAG CATATTAGGA ACAATGAAAA CCTTATTTAC AGTAATTACC AATACTAAAA TATTTTGATG AAATAATCTT CAATCAGAAT AAGTCCAAAT GACAAATTCAT GAAAG) ctcgag ataacttcgtataatgtatgctatacgaagttat atgcatggcc tccgcgccgg gttttggcgc ctcccgcggg cgcccccctc ctcacggcga gcgctgccac gtcagacgaa gggcgcagcg (SEQ ID NO:24).

The nucleotide sequence across the downstream junction point included the following, which indicates cassette sequence (lowercase font with I-CeuI and NheI sites both underlined, and a loxP site in bold font) contiguous with mouse Kynu intron three sequence (uppercase font contained within the parentheses below) downstream of the insertion point: tttcactgca ttctagttgt ggtttgtcca

(SEQ ID NO: 25)
aactcatcaa tgtatcttat catgtctgga ataacttcgtataatgt
atgctatacgaagttat gctag taactataacggtcctaaggtagcga
gctagc (AGCCATTTAA TGTCCAGCAA AGAAGTTAAT
TCATGATTTT GAGTGTTTAA TGATGAATTC ATGACCAAGT
TAAGAATGCC ATCAAAAATA GGAAATACA).

The nucleotide sequence across the insertion point after recombinase-mediated excision of the selection cassette (77 bp remaining in intron three) included the following, which indicates mouse Kynu intron three sequence (uppercase font) juxtaposed with remaining cassette sequence (lowercase font contained within the parentheses below with a XhoI, I-CeuI and NheI sites underlined, and a loxP site in bold font):

(SEQ ID NO: 26)
TTCCTGGGAA ATTCCCTTGG CCTTCAACCG AAAATGGTTA
GGACATACCT GGAGGAAGAG CTTGAAAAAT GGGCTAAGAT
GTAAGTACCA AGTTAAAAGG TGTAACTCCA TCTGACAGAA
GAATTCTGAA AATTACAAAA TGTGTCTGAT TTGGACAAGT
TACACCCTAG CATATTAGGA ACAATGAAAA CCTTATTTAC
AGTAATTACC AATACTAAAA TATTTTGATG AAATAATCTT
CAATCAGAAT AAGTCCAAAT GACAAATTCA TGAAAG (ctcgag
ataacttcgtataatgtatgctatacgaagttat gctag taactataa
cggtcctaaggtagcga gctagc) AGCCATTTAA TGTCCAGCAA
AGAAGTTAATT CATGATTTTG AGTGTTTAAT GATGAATTCA
TGACCAAGTT AAGAATGCCA TCAAAAATAG GAAATACA.

Positive ES cell clones were then used to implant female mice using the VELOCIMOUSE® method (see, e.g., U.S. Pat. No. 7,294,754; DeChiara, T. M. et al., 2010, Methods Enzymol. 476:285-94; DeChiara, T. M., 2009, Methods Mol. Biol. 530:311-24; Poueymirou et al., 2007, Nat. Biotechnol. 25:91-9), in which targeted ES cells were injected into uncompacted 8-cell stage Swiss Webster embryos, to produce healthy fully ES cell-derived F0 generation mice heterozygous for the mutant Kynu gene and that express a Kynu polypeptide containing a D93E amino acid substitution and antibodies having human variable regions and rodent constant regions. F0 generation heterozygous male were crossed with C57B16/NTac females to generate F1 heterozygotes that were intercrossed to produce F2 generation homozygotes and wild-type mice for phenotypic analyses.

Taken together, this example illustrates the generation of a rodent (e.g., a mouse) whose genome comprises a mutant Kynu gene, which mutant Kynu gene comprises one or more point mutations in exon three that results in a D93E substitution. The strategy described herein for generating a mutant Kynu gene in a rodent results in the elimination of a shared epitope between the expressed Kynu polypeptide and the MPER of HIV-1 gp41 and enables the construction of a rodent that expresses antibodies that can be developed for therapeutic treatment of HIV infection. In particular, rodents described herein provide an in vivo system for the production of human antibody-based therapeutics that are characterized by binding to HIV epitopes that are present in endogenous polypeptides and, in some embodiments, are otherwise eliminated from naturally-occurring antibody repertoires due to immunological tolerance mechanisms.

TABLE 2

| Name | Primer | Sequence (5′-3′) |
|---|---|---|
| 4247mTD | Forward | ATGAAAGCGAGAGAGTAAAACAACATAT |
| | | (SEQ ID NO: 27) |
| | Probe | TGTAATCTCCTTTTCTACATCTA |
| | | (SEQ ID NO: 28) |
| | Reverse | GCTGGACATTAAATGGCTACATTG |
| | | (SEQ ID NO: 29) |
| 4247mTU2_D93E | Forward | CCTTGGCCTTCAACCGAAA |
| | | (SEQ ID NO: 30) |
| | Probe | TGGTTAGGACATACCTGGAG |
| | | (SEQ ID NO: 31) |
| | Reverse | TTGGTACTTACATCTTAGCCCATTTTT |
| | | (SEQ ID NO: 32) |

### Example 3. Production of Antibodies that Bind Human Immunodeficiency Virus (HIT)

This example demonstrates production of anti-HIV antibodies in a rodent that comprises a mutant Kynu gene are made using peptides derived from the membrane proximal extended region (MPER) of HIV-1 gp41. In particular, MPER peptides are derived from epitopes of existing anti-HIV antibodies 2F5 and 4E10 and used to immunize mice containing a mutant Kynu gene as described herein using an induction method previously described (see, e.g., Dennison, S. M. et al., 2011, PLos ONE 6(11):e27824). The methods described in this example, or immunization methods well known in the art, can be used to immunize rodents containing a mutant Kynu gene as described above with peptides derived from any epitope present in the MPER of HIV-1 gp41, or combination of epitopes, that is shared with an endogenous polypeptide, as desired.

Human antibodies to the MPER of HIV-1 gp41 are generated using synthetic peptides containing the 2F5 epitope (QQEKNEQELLELDKWASLWN; SEQ ID NO:33) or the epitopes of both 2F5 and 4E10 monoclonal antibodies (NEQELLELDKWASLWNWFNITNWLWYIK; SEQ ID NO:34). Peptides are synthesized (CPC Scientific) with a C-terminal hydrophobic membrane anchor tag (YKRWIILGLNKIVRMYS; SEQ ID NO:35) and purified by reverse phase HPLC. The purity of the MPER peptides is assessed by HPLC to be greater than 95% and confirmed by mass spectrometric analysis.

Cohorts of mice as described in Example 2 (i.e., VELOCIMMUNE® mice that contained a mutant Kynu gene as described above) are challenged with the MPER peptides using methods described previously (Dennison, S. M. et al., supra). The antibody immune response is monitored by an HIV-specific immunoassay (i.e., serum titer). When a desired immune response is achieved, splenocytes (and/or other lymphatic tissue) are harvested and fused with mouse myeloma cells to preserve their viability and form immortal hybridoma cell lines. The hybridoma cell lines are screened (e.g., by an ELISA assay) and selected to identify hybridoma cell lines that produce HIV-specific antibodies. Hybridomas may be further characterized for relative binding affinity and isotype as desired. Using this technique, and the immunogen described above, several anti-HIV chimeric antibodies (i.e., antibodies possessing human variable domains and rodent constant domains) are obtained.

DNA encoding the variable regions of the heavy chain and light chain may be isolated and linked to desirable isotypes (constant regions) of the heavy chain and light chain for the preparation of fully human antibodies. Such an antibody protein may be produced in a cell, such as a CHO cell. Fully human antibodies are then characterized for relative binding affinity and/or neutralizing activity of HIV.

DNA encoding the antigen-specific chimeric antibodies or the variable domains of the light and heavy chains may be isolated directly from antigen-specific lymphocytes. Initially, high affinity chimeric antibodies are isolated having a human variable region and a mouse constant region and are characterized and selected for desirable characteristics, including affinity, selectivity, epitope, etc. Mouse constant regions are replaced with a desired human constant region to generate fully-human antibodies. While the constant region selected may vary according to specific use, high affinity antigen-binding and target specificity characteristics reside in the variable region. Anti-HIV antibodies are also isolated directly from antigen-positive B cells (from immunized mice) without fusion to myeloma cells, as described in U.S. Pat. No. 7,582,298, specifically incorporated herein by reference in its entirety. Using this method, several fully human anti-HIV antibodies (i.e., antibodies possessing human variable domains and human constant domains) are made.

### EQUIVALENTS

Having thus described several aspects of at least one embodiment of this invention, it is to be appreciated by those skilled in the art that various alterations, modifications, and improvements will readily occur to those skilled in the art. Such alterations, modifications, and improvements are intended to be part of this disclosure, and are intended to be within the spirit and scope of the invention. Accordingly, the foregoing description and drawing are by way of example only and the invention is described in detail by the claims that follow.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

Those skilled in the art will appreciate typical standards of deviation or error attributable to values obtained in assays or other processes described herein. The publications, websites and other reference materials referenced herein to describe the background of the invention and to provide additional detail regarding its practice are hereby incorporated by reference.

1. A rodent whose genome comprises a mutant kynureninase (Kynu) gene, which mutant Kynu gene comprises one or more point mutations in exon three and encodes a Kynu polypeptide having a D93E substitution.

2. The rodent of claim 1, wherein the mutant Kynu gene comprises 5 point mutations in exon three.

3. The rodent of claim 1, wherein the mutant Kynu gene further comprises one or more selection markers.

4. The rodent of claim 1, wherein the mutant Kynu gene further comprises one or more site-specific recombinase recognition sites.

5. The rodent of claim 4, wherein the mutant Kynu gene comprises a recombinase gene and a selection marker flanked by recombinase recognition sites, which recombinase recognition sites are oriented to direct an excision.

6. The rodent of claim 5, wherein the recombinase gene is operably linked to a promoter that drives expression of the recombinase gene in differentiated cells and does not drive expression of the recombinase gene in undifferentiated cells, or is transcriptionally competent and developmentally regulated.

7. The rodent of claim 6, wherein the promoter is or comprises SEQ ID NO:37, SEQ ID NO:38, or SEQ ID NO:39.

8. The rodent of claim 7, wherein the promoter is or comprises SEQ ID NO:37.

9. The rodent of claim 1, wherein the mutant Kynu gene comprises an exon three nucleic acid sequence comprising SEQ ID NO:42 or encoding a Kynu polypeptide comprising an amino acid sequence comprising SEQ ID NO:41.

10. The rodent of claim 1, wherein the genome of the rodent further comprises an insertion of a human immunoglobulin heavy chain variable region that includes one or more human V_{H} segments, one or more human D_{H} segments and one or more human J_{H} segments, which human immunoglobulin heavy chain variable region is operably linked to an immunoglobulin heavy chain constant region.

11. The rodent of claim 10, wherein the immunoglobulin heavy chain constant region is a rodent immunoglobulin heavy chain constant region.

12. The rodent of claim 11, wherein the rodent immunoglobulin heavy chain constant region is an endogenous rodent immunoglobulin heavy chain constant region.

13. The rodent of claim 1, wherein the genome of the rodent further comprises an insertion of a human immunoglobulin light chain variable region that includes one or more human V_{L} segments and one or more human J_{L} segments, which human immunoglobulin light chain variable region is operably linked to an immunoglobulin light chain constant region.

14. The rodent of claim 10, wherein the genome of the rodent further comprises an insertion of a human immunoglobulin light chain variable region that includes one or more human V_{L} segments and one or more human J_{L} segments, which human immunoglobulin light chain variable region is operably linked to an immunoglobulin light chain constant region.

15. The rodent of claim 13, wherein the immunoglobulin light chain constant region is a rodent immunoglobulin light chain constant region.

16. The rodent of claim 15, wherein the rodent immunoglobulin light chain constant region is an endogenous rodent immunoglobulin light chain constant region.

17. The rodent of claim 13, wherein the human V_{L} and J_{L} segments are human Vκ and Jκ segments and are inserted into an endogenous κ light chain locus.

18. The rodent of claim 17, wherein the human Vκ and Jκ segments are operably linked to a rodent Cκ gene.

19. The rodent of claim 13, wherein the human V_{L} and J_{L} segments are human Vλ and Jλ segments and are inserted into an endogenous λ light chain locus.

20. The rodent of claim 19, wherein the human Vλ and Jλ segments are operably linked to a rodent Cλ gene.

21-27. (canceled)

28. An isolated rodent cell or tissue derived from the rodent of claim 1.

29. An immortalized cell made from the isolated rodent cell of claim 28.

30. The cell of claim 28, wherein the cell is an embryonic stem cell.

31-46. (canceled)

47. A method of making a rodent whose genome comprises a mutant kynureninase (Kynu) gene, which mutant Kynu gene encodes a Kynu polypeptide that includes a D93E substitution, the method comprising
modifying the genome of a rodent so that it comprises a mutant Kynu gene that encodes a Kynu polypeptide having a D93E substitution, thereby making said rodent.

48-62. (canceled)

63. A method of producing an antibody in a rodent, the method comprising the steps of
(a) immunizing a rodent with an antigen, which rodent has a genome comprising a mutant kynureninase (Kynu) gene that encodes a Kynu polypeptide having a D93E substitution;
(b) maintaining the rodent under conditions sufficient that the rodent produces an immune response to the antigen; and
(c) recovering an antibody from the rodent, or a rodent cell, that binds the antigen.

64-78. (canceled)

79. A rodent whose genome comprises
(i) a mutant Kynu gene, which mutant kynureninase (Kynu) gene comprises one or more point mutations in exon three and encodes a Kynu polypeptide having a D93E substitution;
(ii) an insertion of a human immunoglobulin heavy chain variable region that includes one or more human V_{H} segments, one or more human D_{H} segments and one or more human J_{H} segments, which human immunoglobulin heavy chain variable region is operably linked to an endogenous rodent immunoglobulin heavy chain constant region; and
(ii) an insertion of a human immunoglobulin light chain variable region that includes one or more human V_{L} segments and one or more human J_{L} segments, which human immunoglobulin light chain variable region is operably linked to an endogenous rodent immunoglobulin light chain constant region.

80-84. (canceled)

85. A method of producing an antibody in a rodent, the method comprising the steps of
(a) immunizing a rodent with the membrane proximal external region (MPER) of HIV-1 gp4, in whole or in part, which rodent has a genome comprising
(i) a mutant Kynu gene that includes one or more point mutations in exon three and encodes a Kynu polypeptide having a D93E substitution;
(ii) an insertion of a human immunoglobulin heavy chain variable region that includes one or more human V_{H} segments, one or more human D_{H} segments and one or more human J_{H} segments, which human immunoglobulin heavy chain variable region is operably linked to an endogenous rodent immunoglobulin heavy chain constant region; and
(ii) an insertion of a human immunoglobulin light chain variable region that includes one or more human V_{L} segments and one or more human J_{L} segments, which human immunoglobulin light chain variable region is operably linked to an endogenous rodent immunoglobulin light chain constant region.
(b) maintaining the rodent under conditions sufficient that the rodent produces an immune response to the MPER of HIV-1 gp41, in whole or in part; and
(c) recovering an antibody from the rodent, or a rodent cell, that binds the MPER of HIV-1 gp41;
wherein the antibody comprises immunoglobulin heavy chains that include human V_{H} domains linked to rodent C_{H} domains, and immunoglobulin light chains that include human Vκ domains linked to rodent Cκ domains.

86-90. (canceled)


SEQUENCE LISTING (43 sequences)

SEQ ID NO: 1 - DNA, 1688 bp, Homo sapiens
gcagttctttgaatttctcaccctaagatctggcctgtacattttcaaggaattcttgagaggttcttggagagattctgggagccaaacactccattgggatcctagctgttttagagaacaacttgtaatggagccttcatctcttgagctgccggctgacacagtgcagcgcattgcggctgaactcaaatgccacccaacggatgagagggtggctctccacctagatgaggaagataagctgaggcacttcagggagtgcttttatattcccaaaatacaggatctgcctccagttgatttatcattagtgaataaagatgaaaatgccatctatttcttgggaaattctcttggccttcaaccaaaaatggttaaaacatatcttgaagaagaactagataagtgggccaaaatagcagcctatggtcatgaagtggggaagcgtccttggattacaggagatgagagtattgtaggccttatgaaggacattgtaggagccaatgagaaagaaatagccctaatgaatgctttgactgtaaatttacatcttctaatgttatcattttttaagcctacgccaaaacgatataaaattcttctagaagccaaagccttcccttctgatcattatgctattgagtcacaactacaacttcacggacttaacattgaagaaagtatgcggatgataaagccaagagagggggaagaaaccttaagaatagaggatatccttgaagtaattgagaaggaaggagactcaattgcagtgatcctgttcagtggggtgcatttttacactggacagcactttaatattcctgccatcacaaaagctggacaagcgaagggttgttatgttggctttgatctagcacatgcagttggaaatgttgaactctacttacatgactggggagttgattttgcctgctggtgttcctacaagtatttaaatgcaggagcaggaggaattgctggtgccttcattcatgaaaagcatgcccatacgattaaacctgcattagtgggatggtttggccatgaactcagcaccagatttaagatggataacaaactgcagttaatccctggggtctgtggattccgaatttcaaatcctcccattttgttggtctgttccttgcatgctagtttagagatctttaagcaagcgacaatgaaggcattgcggaaaaaatctgttttgctaactggctatctggaatacctgatcaagcataactatggcaaagataaagcagcaaccaagaaaccagttgtgaacataattactccgtctcatgtagaggagcgggggtgccagctaacaataacattttctgttccaaacaaagatgttttccaagaactagaaaaaagaggagtggtttgtgacaagcggaatccaaatggcattcgagtggctccagttcctctctataattctttccatgatgtttataaatttaccaatctgctcacttctatacttgactctgcagaaacaaaaaattagcagtgttttctagaacaacttaagcaaattatactgaaagctgctgtggttatttcagtattattcgatttttaattattgaaagtatgtcaccattgaccacatgtaactaacaataaataatataccttacagaaaatctgaaaaaaaaaaaaaaaaa

SEQ ID NO: 2 - protein, 465 aa, Homo sapiens
MEPSSLELPADTVQRIAAELKCHPTDERVALHLDEEDKLRHFRECFYIPKIQDLPPVDLSLVNKDENAIYFLGNSLGLQPKMVKTYLEEELDKWAKIAAYGHEVGKRPWITGDESIVGLMKDIVGANEKEIALMNALTVNLHLLMLSFFKPTPKRYKILLEAKAFPSDHYAIESQLQLHGLNIEESMRMIKPREGEETLRIEDILEVIEKEGDSIAVILFSGVHFYTGQHFNIPAITKAGQAKGCYVGFDLAHAVGNVELYLHDWGVDFACWCSYKYLNAGAGGIAGAFIHEKHAHTIKPALVGWFGHELSTRFKMDNKLQLIPGVCGFRISNPPILLVCSLHASLEIFKQATMKALRKKSVLLTGYLEYLIKHNYGKDKAATKKPVVNIITPSHVEERGCQLTITFSVPNKDVFQELEKRGVVCDKRNPNGIRVAPVPLYNSFHDVYKFTNLLTSILDSAETKN

SEQ ID NO: 3 - DNA, 3070 bp, Mus musculus
gagcagttctttggctagctggggacaaagaaagatccagcatcctctgagaaggtactgaagactactgtctggatctgagcagataacagtttatgatggagccttcgcctcttgagcttccagttgatgcagtgcggcgcatcgcggctgaactcaattgtgacccaacagatgagagggttgctctccgcttggatgaggaagataaactgagtcattttaggaactgtttttatattcccaaaatgcgggacctgccttcaattgatctatctttagtgagtgaggatgatgatgccatctatttcctgggaaattcccttggccttcaaccgaaaatggttaggacatacctggaggaagaactagataagtgggccaagatgggagcctatggccatgatgtaggcaaacgcccttggattgtaggggatgagagtattgtaagccttatgaaggacattgtaggagcccatgagaaagaaatagctctaatgaatgctttgactattaatttacatctcctgctgttatcattctttaagcctactccaaagcggcacaaaattcttctagaagccaaagccttcccttctgatcattatgctattgagtcacagattcaacttcacggacttgatgttgagaaaagtatgcggatggtaaagccacgagagggggaagagaccttaaggatggaggacatactggaagtaatcgaggaggaaggagactcgatcgccgtgatcctgttcagtgggctgcacttttatactggacagctgttcaacattcctgccataacaaaagctggacatgcaaagggctgttttgttggctttgacctagcacatgcagttggaaatgttgaactccgcttacatgactggggtgttgactttgcctgctggtgttcctataagtatttaaattcaggagctggaggtctggctggtgcctttgtccacgagaaacatgctcatactgtcaagcctgcgttagtgggatggttcggccatgacctcagtacaaggtttaacatggataacaaactacaattaatccccggggccaatggattccgaatttcaaaccctcccattttgttggtctgctccttgcacgccagtttagaggtctttcagcaagcaactatgactgcgctgagaagaaaatccattctgctgacaggttatctggaatacatgctcaaacattaccacagcaaagataacaccgaaaacaaggggccgattgtgaatatcatcaccccgtccagagcagaggagcgtggctgccagttaacactcaccttttccattcccaagaaaagcgtttttaaggaactagaaaaaagaggagtcgtttgtgacaagcgagaaccagatggcatccgcgtggcccctgttcctctctataattctttccatgatgtttataagttcatcagactgctcacttccatactcgactcttcagaaagaagctagctatattttctagcacaactcaagtaaatctcactgaaaggtgatggagttttcacttctattgaattttagtcattaaaaaaatctccagaaattgattgcacagaaatgataactataaaaaaatttacataaaacctggtgcatgctttaatatctgtgtttctggggaacgtggtgtcctgtgaattatgaagtcacactttacatgactacagcctacagatgactgtcttgatcagttgtcacatttcatgctcactgaaacattttctctttaatttgtgactgaatttccaacgttataatgtatatggacttcttgtataaatattagaagtattactttaattttgctatagagttttattttaatatttgtaactgaatcatctgaaatatgtttgatatgatcatgttttatctaattccaggaggggaacagccttttaagctgttacaaaatctctctctctctctctctctctctctctctctctctctctctctctctctctctctctctctttcccccccccagtggtgtgtgtgtctatgtgtttgtgtttctgtgtgtctgtgtaaaggacatgtaagtgcttatgtataagggatgaggtacttgaccctatgtactcttgtgaggccagaggtcaacactggacatcttcctcaatcactgtttaaaattttatttatttatttatttttatgtgtatgggtattttgactgcatttatgtctgtacctcatgtgcatgccatgattacagaaactagaagatacatcagatcacctgagactggagttacagagctgctgtgtggatactaggaattgaacccaggtcgtttggaagaatagccaacgctcttattctttgacacatctctccagcctttccacttcatatttcaatacatgatttctccccaaacctggaacttgctccttcagctcgtgtggctggccagtgagtcttcagcgtttctctgtctctgccctacaatgaatgcgggttacagctgtacactgttgcacatagattttttacatgtctactgtgatctgaacacagtccttatatcagttcagcaaccacttcatcgaccaagcaatcccccagtcattgcttttttgatgccactactagtatgcatttactggcaaagaattctaagtttgtatgtagaaagaaaaagttataatgatttgataaacttgaataaaacatacttggtcagacagaaacttctgatgtgataaatgataagatatggaactctggcagtagctaacaacaaacacagcactcttgtttacttaggaattcaattccgagtgttgcacacatatctatgttaacatagcaaagctttccactgcattatttcaccttcattaatgaaatggctatcaggacctggaaactcatccgtaacacagattcctacatgactgtttttgagtcccacagtggtcaacaaaaggacatggttttcattttcaaggaacagagtaccctggtgccattcttcattgcaaaaaatataaaaataaaataaatagttaattat

SEQ ID NO: 4 - protein, 465 aa, Mus musculus
MMEPSPLELPVDAVRRIAAELNCDPTDERVALRLDEEDKLSHFRNCFYIPKMRDLPSIDLSLVSEDDDAIYFLGNSLGLQPKMVRTYLEEELDKWAKMGAYGHDVGKRPWIVGDESIVSLMKDIVGAHEKEIALMNALTINLHLLLLSFFKPTPKRHKILLEAKAFPSDHYAIESQIQLHGLDVEKSMRMVKPREGEETLRMEDILEVIEEEGDSIAVILFSGLHFYTGQLFNIPAITKAGHAKGCFVGFDLAHAVGNVELRLHDWGVDFACWCSYKYLNSGAGGLAGAFVHEKHAHTVKPALVGWFGHDLSTRFNMDNKLQLIPGANGFRISNPPILLVCSLHASLEVFQQATMTALRRKSILLTGYLEYMLKHYHSKDNTENKGPIVNIITPSRAEERGCQLTLTFSIPKKSVFKELEKRGVVCDKREPDGIRVAPVPLYNSFHDVYKFIRLLTSILDSSERS

SEQ ID NO: 5 - DNA, 1764 bp, Rattus norvegicus
tgaaaaggtactggaaactgaggaccctatctggatcaaagcagtttctgatggagccctcgcctcttgagctaccagttgatgcagtgcggcgcatcgcggctgaactcaattgtgacccaaccgatgagagggtggctctccgcttggatgaggaagataaactgaagcgttttaaggactgtttttatatccccaaaatgcgggacctgccttcaattgatctatctttagtgaatgaggatgataatgccatctatttcctgggaaattcccttggtcttcaaccgaagatggttaaaacatacctggaggaagagctagataagtgggccaaaataggagcctatggccatgaggtagggaaacgtccttggattataggagatgagagcattgtaacccttatgaaggacattgtaggagcccatgagaaagaaatagctctaatgaatgctttgactgttaatttacatctcctgctgttatcattctttaagcctacaccaaagcggcacaaaattcttctagaagccaaagccttcccttctgatcattatgcgatcgagtcacagattcaacttcatggacttgatgttgagaaaagtatgcggatgataaagccacgagagggggaagagaccttaagaatggaggacatactggaagtaattgagaaggaaggagactcaattgctgtggtcctgttcagtggcctgcacttttatactggacagctgttcaacattcctgccattacacaagccggacatgcaaagggctgttttgttggctttgacctagcgcatgcggttggaaatgttgaactccacttacatgactgggatgttgactttgcctgctggtgctcctacaagtatttaaattcaggagctggaggtctggctggtgccttcatccatgagaaacacgctcacacgatcaagccagcgttagtgggatggttcggccatgaactcagtacaagatttaacatggataacaaactacaattaatccccggggtcaatggattccgaatttccaaccctcccattctgttggtctgctccttgcatgccagtttagagatctttcagcaagcaactatgactgcgctgaggagaaaatccattctgctgacaggttatctggaatacttgctcaaacattaccatggcggaaatgacacagaaaacaagaggccagttgtgaacataatcaccccatccagagcagaggaacgaggctgccagctgacactgaccttttccatttccaagaaaggcgtttttaaggaactagaaaaaagaggagtcgtctgtgacaagcgagaaccagaaggcatccgggtggccccggttcctctctataattctttccatgatgtttataagttcatcagactgcttactgccatactcgactctacagaaagaaactagccatgctttctaaataactcaagtaaatctcacacactgggggttccacttctactgcattttagtcattcaaaagtctccagaaattgatggcatagaaatgatgatgattttataaacttacataaaacctggtacatgttttaatatctgtgtcgctgatgtgctgtggactaagaagtcacattttacatgactccaacctacagatgactgtcttgatcagctgtcaccttccatggtcactgaaaggttgtgtgtttaatttgtgactgaaatgacaacattaaaatgtatctggacttcttgtataaaaaaa

SEQ ID NO: 6 - protein, 464 aa, Rattus norvegicus
MEPSPLELPVDAVRRIAAELNCDPTDERVALRLDEEDKLKRFKDCFYIPKMRDLPSIDLSLVNEDDNAIYFLGNSLGLQPKMVKTYLEEELDKWAKIGAYGHEVGKRPWIIGDESIVTLMKDIVGAHEKEIALMNALTVNLHLLLLSFFKPTPKRHKILLEAKAFPSDHYAIESQIQLHGLDVEKSMRMIKPREGEETLRMEDILEVIEKEGDSIAVVLFSGLHFYTGQLFNIPAITQAGHAKGCFVGFDLAHAVGNVELHLHDWDVDFACWCSYKYLNSGAGGLAGAFIHEKHAHTIKPALVGWFGHELSTRFNMDNKLQLIPGVNGFRISNPPILLVCSLHASLEIFQQATMTALRRKSILLTGYLEYLLKHYHGGNDTENKRPVVNIITPSRAEERGCQLTLTFSISKKGVFKELEKRGVVCDKREPEGIRVAPVPLYNSFHDVYKFIRLLTAILDSTERN

SEQ ID NO: 7 - DNA, 3070 bp, Mus musculus
gagcagttctttggctagctggggacaaagaaagatccagcatcctctgagaaggtactgaagactactgtctggatctgagcagataacagtttatgatggagccttcgcctcttgagcttccagttgatgcagtgcggcgcatcgcggctgaactcaattgtgacccaacagatgagagggttgctctccgcttggatgaggaagataaactgagtcattttaggaactgtttttatattcccaaaatgcgggacctgccttcaattgatctatctttagtgagtgaggatgatgatgccatctatttcctgggaaattcccttggccttcaaccgaaaatggttaggacatacctggaggaagagcttgaaaaatgggctaagatgggagcctatggccatgatgtaggcaaacgcccttggattgtaggggatgagagtattgtaagccttatgaaggacattgtaggagcccatgagaaagaaatagctctaatgaatgctttgactattaatttacatctcctgctgttatcattctttaagcctactccaaagcggcacaaaattcttctagaagccaaagccttcccttctgatcattatgctattgagtcacagattcaacttcacggacttgatgttgagaaaagtatgcggatggtaaagccacgagagggggaagagaccttaaggatggaggacatactggaagtaatcgaggaggaaggagactcgatcgccgtgatcctgttcagtgggctgcacttttatactggacagctgttcaacattcctgccataacaaaagctggacatgcaaagggctgttttgttggctttgacctagcacatgcagttggaaatgttgaactccgcttacatgactggggtgttgactttgcctgctggtgttcctataagtatttaaattcaggagctggaggtctggctggtgcctttgtccacgagaaacatgctcatactgtcaagcctgcgttagtgggatggttcggccatgacctcagtacaaggtttaacatggataacaaactacaattaatccccggggccaatggattccgaatttcaaaccctcccattttgttggtctgctccttgcacgccagtttagaggtctttcagcaagcaactatgactgcgctgagaagaaaatccattctgctgacaggttatctggaatacatgctcaaacattaccacagcaaagataacaccgaaaacaaggggccgattgtgaatatcatcaccccgtccagagcagaggagcgtggctgccagttaacactcaccttttccattcccaagaaaagcgtttttaaggaactagaaaaaagaggagtcgtttgtgacaagcgagaaccagatggcatccgcgtggcccctgttcctctctataattctttccatgatgtttataagttcatcagactgctcacttccatactcgactcttcagaaagaagctagctatattttctagcacaactcaagtaaatctcactgaaaggtgatggagttttcacttctattgaattttagtcattaaaaaaatctccagaaattgattgcacagaaatgataactataaaaaaatttacataaaacctggtgcatgctttaatatctgtgtttctggggaacgtggtgtcctgtgaattatgaagtcacactttacatgactacagcctacagatgactgtcttgatcagttgtcacatttcatgctcactgaaacattttctctttaatttgtgactgaatttccaacgttataatgtatatggacttcttgtataaatattagaagtattactttaattttgctatagagttttattttaatatttgtaactgaatcatctgaaatatgtttgatatgatcatgttttatctaattccaggaggggaacagccttttaagctgttacaaaatctctctctctctctctctctctctctctctctctctctctctctctctctctctctctctctttcccccccccagtggtgtgtgtgtctatgtgtttgtgtttctgtgtgtctgtgtaaaggacatgtaagtgcttatgtataagggatgaggtacttgaccctatgtactcttgtgaggccagaggtcaacactggacatcttcctcaatcactgtttaaaattttatttatttatttatttttatgtgtatgggtattttgactgcatttatgtctgtacctcatgtgcatgccatgattacagaaactagaagatacatcagatcacctgagactggagttacagagctgctgtgtggatactaggaattgaacccaggtcgtttggaagaatagccaacgctcttattctttgacacatctctccagcctttccacttcatatttcaatacatgatttctccccaaacctggaacttgctccttcagctcgtgtggctggccagtgagtcttcagcgtttctctgtctctgccctacaatgaatgcgggttacagctgtacactgttgcacatagattttttacatgtctactgtgatctgaacacagtccttatatcagttcagcaaccacttcatcgaccaagcaatcccccagtcattgcttttttgatgccactactagtatgcatttactggcaaagaattctaagtttgtatgtagaaagaaaaagttataatgatttgataaacttgaataaaacatacttggtcagacagaaacttctgatgtgataaatgataagatatggaactctggcagtagctaacaacaaacacagcactcttgtttacttaggaattcaattccgagtgttgcacacatatctatgttaacatagcaaagctttccactgcattatttcaccttcattaatgaaatggctatcaggacctggaaactcatccgtaacacagattcctacatgactgtttttgagtcccacagtggtcaacaaaaggacatggttttcattttcaaggaacagagtaccctggtgccattcttcattgcaaaaaatataaaaataaaataaatagttaattat

SEQ ID NO: 8 - protein, 465 aa, Mus musculus
MMEPSPLELPVDAVRRIAAELNCDPTDERVALRLDEEDKLSHFRNCFYIPKMRDLPSIDLSLVSEDDDAIYFLGNSLGLQPKMVRTYLEEELEKWAKMGAYGHDVGKRPWIVGDESIVSLMKDIVGAHEKEIALMNALTINLHLLLLSFFKPTPKRHKILLEAKAFPSDHYAIESQIQLHGLDVEKSMRMVKPREGEETLRMEDILEVIEEEGDSIAVILFSGLHFYTGQLFNIPAITKAGHAKGCFVGFDLAHAVGNVELRLHDWGVDFACWCSYKYLNSGAGGLAGAFVHEKHAHTVKPALVGWFGHDLSTRFNMDNKLQLIPGANGFRISNPPILLVCSLHASLEVFQQATMTALRRKSILLTGYLEYMLKHYHSKDNTENKGPIVNIITPSRAEERGCQLTLTFSIPKKSVFKELEKRGVVCDKREPDGIRVAPVPLYNSFHDVYKFIRLLTSILDSSERS

SEQ ID NO: 9 - DNA, 8630 bp, Mus musculus
taatggtggactctgtagaaggctgatattctgcagaaaaaaaaatgatgatggctacattatttcaacgttttacttccttcttagataacagtttatgggtaccgatttaaatgatccagtggtcctgcagaggagagattgggagaatcccggtgtgacacagctgaacagactagccgcccaccctccctttgcttcttggagaaacagtgaggaagctaggacagacagaccaagccagcaactcagatctttgaacggggagtggagatttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatccccgggctcgataactataacggtcctaaggtagcgactcgacataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgagctcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctagcgagaggtatctgtgaaagaaagaaatgctcattagacttccattttgtgttcacttatgtccctcaaaagtatattatcttcatggctctgatgtaacaa

SEQ ID NO: 10 - DNA, 8852 bp, Mus musculus
taatggtggactctgtagaaggctgatattctgcagaaaaaaaaatgatgatggctacattatttcaacgttttacttccttcttagataacagtttatgggtaccgatttaaatgatccagtggtcctgcagaggagagattgggagaatcccggtgtgacacagctgaacagactagccgcccaccctccctttgcttcttggagaaacagtgaggaagctaggacagacagaccaagccagcaactcagatctttgaacggggagtggagatttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatccccgggctcgataactataacggtcctaaggtagcgactcgacataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgagctcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctagcgagaggtatctgtgaaagaaagaaatgctcattagacttccattttgtgttcacttatgtccctcaaaagtatattatcttcatggctctgatgtaacaa

SEQ ID NO: 11 - DNA, 3670 bp, Mus musculus
taatggtggactctgtagaaggctgatattctgcagaaaaaaaaatgatgatggctacattatttcaacgttttacttccttcttagataacagtttatgggtaccgatttaaatgatccagtggtcctgcagaggagagattgggagaatcccggtgtgacacagctgaacagactagccgcccaccctccctttgcttcttggagaaacagtgaggaagctaggacagacagaccaagccagcaactcagatctttgaacggggagtggagatttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatccccgggctcgataactataacggtcctaaggtagcgactcgacataacttcgtataatgtatgctatacgaagttatgctagcgagaggtatctgtgaaagaaagaaatgctcattagacttccattttgtgttcacttatgtccctcaaaagtatattatcttcatggctctgatgtaacaa

SEQ ID NO: 12 - DNA, 5784 bp, Mus musculus
agagcctgaggcttctgtgggagtaactgcaagttatttattacccttcctcttgtaaattatgttaataacgctggattaacaatgacaactgggagaatgttaattaatttaacaagcacttttttttttgtattttcttgtttcagttgatctatctttagtgagtgaggatgatgatgccatctatttcctgggaaattcccttggccttcaaccgaaaatggttaggacatacctggaggaagagcttgaaaaatgggctaagatgtaagtaccaagttaaaaggtgtaactccatctgacagaagaattctgaaaattacaaaatgtgtctgatttggacaagttacaccctagcatattaggaacaatgaaaaccttatttacagtaattaccaatactaaaatattttgatgaaataatcttcaatcagaataagtccaaatgacaaattcatgaaagctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagcagccatttaatgtccagcaaagaagttaattcatgattttgagtgtttaatgatgaattcatgaccaagttaagaatgccatcaaaaataggaaatacag

SEQ ID NO: 13 - DNA, 5562 bp, Mus musculus
agagcctgaggcttctgtgggagtaactgcaagttatttattacccttcctcttgtaaattatgttaataacgctggattaacaatgacaactgggagaatgttaattaatttaacaagcacttttttttttgtattttcttgtttcagttgatctatctttagtgagtgaggatgatgatgccatctatttcctgggaaattcccttggccttcaaccgaaaatggttaggacatacctggaggaagagcttgaaaaatgggctaagatgtaagtaccaagttaaaaggtgtaactccatctgacagaagaattctgaaaattacaaaatgtgtctgatttggacaagttacaccctagcatattaggaacaatgaaaaccttatttacagtaattaccaatactaaaatattttgatgaaataatcttcaatcagaataagtccaaatgacaaattcatgaaagctcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagcagccatttaatgtccagcaaagaagttaattcatgattttgagtgtttaatgatgaattcatgaccaagttaagaatgccatcaaaaataggaaatacag

SEQ ID NO: 14 - DNA, 643 bp, Mus musculus
agagcctgaggcttctgtgggagtaactgcaagttatttattacccttcctcttgtaaattatgttaataacgctggattaacaatgacaactgggagaatgttaattaatttaacaagcacttttttttttgtattttcttgtttcagttgatctatctttagtgagtgaggatgatgatgccatctatttcctgggaaattcccttggccttcaaccgaaaatggttaggacatacctggaggaagagcttgaaaaatgggctaagatgtaagtaccaagttaaaaggtgtaactccatctgacagaagaattctgaaaattacaaaatgtgtctgatttggacaagttacaccctagcatattaggaacaatgaaaaccttatttacagtaattaccaatactaaaatattttgatgaaataatcttcaatcagaataagtccaaatgacaaattcatgaaagctcgagataacttcgtataatgtatgctatacgaagttatgctagtaactataacggtcctaaggtagcgagctagcagccatttaatgtccagcaaagaagttaattcatgattttgagtgtttaatgatgaattcatgaccaagttaagaatgccatcaaaaataggaaatacag

SEQ ID NO: 15 - DNA, 75 bp, Mus musculus
ttttacttccttcttagataacagtttatgggtaccgatttaaatgatccagtggtcctgcagaggagagattgg

SEQ ID NO: 16 - DNA, 79 bp, Mus musculus
ataacttcgtataatgtatgctatacgaagttatgctagcgagaggtatctgtgaaagaaagaaatgctcattagactt

SEQ ID NO: 17 - DNA, 179 bp, Mus musculus
ctcatcaatgtatcttatcatgtctggatcccccggctagagtttaaacactagaactagtggatccccgggctaactataacggtcctaaggtagcgactcgacataacttcgtataatgtatgctatacgaagttatgctagcgagaggtatctgtgaaagaaagaaatgctcatta

SEQ ID NO: 18 - DNA, 20 bp, Mus musculus
tgctaccctaccaacccatc

SEQ ID NO: 19 - DNA, 26 bp, Mus musculus
cctacccgagcctcgtgttctttacg

SEQ ID NO: 20 - DNA, 22 bp, Mus musculus
gacagcgtaaacaccctgagag

SEQ ID NO: 21 - DNA, 24 bp, Mus musculus
attctgcacttctgatcaccttta

SEQ ID NO: 22 - DNA, 29 bp, Mus musculus
tcaacaagtaccctgattcacattaagga

SEQ ID NO: 23 - DNA, 22 bp, Mus musculus
gaatggctacctcacagacatc

SEQ ID NO: 24 - DNA, 406 bp, Mus musculus
ttcctgggaaattcccttggccttcaaccgaaaatggttaggacatacctggaggaagagcttgaaaaatgggctaagatgtaagtaccaagttaaaaggtgtaactccatctgacagaagaattctgaaaattacaaaatgtgtctgatttggacaagttacaccctagcatattaggaacaatgaaaaccttatttacagtaattaccaatactaaaatattttgatgaaataatcttcaatcagaataagtccaaatgacaaattcatgaaagctcgagataacttcgtataatgtatgctatacgaagttatatgcatggcctccgcgccgggttttggcgcctcccgcgggcgcccccctcctcacggcgagcgctgccacgtcagacgaagggcgcagcg

SEQ ID NO: 25 - DNA, 230 bp, Mus musculus
tttcactgcattctagttgtggtttgtccaaactcatcaatgtatcttatcatgtctggaataacttcgtataatgtatgctatacgaagttatgctagtaactataacggtcctaaggtagcgagctagcagccatttaatgtccagcaaagaagttaattcatgattttgagtgtttaatgatgaattcatgaccaagttaagaatgccatcaaaaataggaaataca

SEQ ID NO: 26 - DNA, 452 bp, Mus musculus
ttcctgggaaattcccttggccttcaaccgaaaatggttaggacatacctggaggaagagcttgaaaaatgggctaagatgtaagtaccaagttaaaaggtgtaactccatctgacagaagaattctgaaaattacaaaatgtgtctgatttggacaagttacaccctagcatattaggaacaatgaaaaccttatttacagtaattaccaatactaaaatattttgatgaaataatcttcaatcagaataagtccaaatgacaaattcatgaaagctcgagataacttcgtataatgtatgctatacgaagttatgctagtaactataacggtcctaaggtagcgagctagcagccatttaatgtccagcaaagaagttaattcatgattttgagtgtttaatgatgaattcatgaccaagttaagaatgccatcaaaaataggaaataca

SEQ ID NO: 27 - DNA, 28 bp, Mus musculus
atgaaagcgagagagtaaaacaacatat

SEQ ID NO: 28 - DNA, 23 bp, Mus musculus
tgtaatctccttttctacatcta

SEQ ID NO: 29 - DNA, 24 bp, Mus musculus
gctggacattaaatggctacattg

SEQ ID NO: 30 - DNA, 19 bp, Mus musculus
ccttggccttcaaccgaaa

SEQ ID NO: 31 - DNA, 20 bp, Mus musculus
tggttaggacatacctggag

SEQ ID NO: 32 - DNA, 27 bp, Mus musculus
ttggtacttacatcttagcccattttt

SEQ ID NO: 33 - protein, 20 aa, Homo sapiens
QQEKNEQELLELDKWASLWN

SEQ ID NO: 34 - protein, 28 aa, Homo sapiens
NEQELLELDKWASLWNWFNITNWLWYIK

SEQ ID NO: 35 - protein, 17 aa, Homo sapiens
YKRWIILGLNKIVRMYS

SEQ ID NO: 36 - protein, 6 aa, Artificial Sequence
  Synthetic construct
ELEKWA

SEQ ID NO: 37 - DNA, 680 bp, Mus musculus
ccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcacc

SEQ ID NO: 38 - DNA, 1052 bp, Mus musculus
tgccatcatcacaggatgtccttccttctccagaagacagactggggctgaaggaaaagccggccaggctcagaacgagccccactaattactgcctccaacagctttccactcactgcccccagcccaacatcccctttttaactgggaagcattcctactctccattgtacgcacacgctcggaagcctggctgtgggtttgggcatgagaggcagggacaacaaaaccagtatatatgattataactttttcctgtttccctatttccaaatggtcgaaaggaggaagttaggtctacctaagctgaatgtattcagttagcaggagaaatgaaatcctatacgtttaatactagaggagaaccgccttagaatatttatttcattggcaatgactccaggactacacagcgaaattgtattgcatgtgctgccaaaatactttagctctttccttcgaagtacgtcggatcctgtaattgagacaccgagtttaggtgactagggttttcttttgaggaggagtcccccaccccgccccgctctgccgcgacaggaagctagcgatccggaggacttagaatacaatcgtagtgtgggtaaacatggagggcaagcgcctgcaaagggaagtaagaagattcccagtccttgttgaaatccatttgcaaacagaggaagctgccgcgggtcgcagtcggtggggggaagccctgaaccccacgctgcacggctgggctggccaggtgcggccacgcccccatcgcggcggctggtaggagtgaatcagaccgtcagtattggtaaagaagtctgcggcagggcagggagggggaagagtagtcagtcgctcgctcactcgctcgctcgcacagacactgctgcagtgacactcggccctccagtgtcgcggagacgcaagagcagcgcgcagcacctgtccgcccggagcgagcccggcccgcggccgtagaaaaggagggaccgccgaggtgcgcgtcagtactgctcagcccggcagggacgcgggaggatgtggactgggtggac

SEQ ID NO: 39 - DNA, 2008 bp, Mus musculus
gtggtgctgactcagcatcggttaataaaccctctgcaggaggctggatttcttttgtttaattatcacttggacctttctgagaactcttaagaattgttcattcgggtttttttgttttgttttggtttggtttttttgggttttttttttttttttttttttggtttttggagacagggtttctctgtatatagccctggcacaagagcaagctaacagcctgtttcttcttggtgctagcgccccctctggcagaaaatgaaataacaggtggacctacaacccccccccccccccccagtgtattctactcttgtccccggtataaatttgattgttccgaactacataaattgtagaaggattttttagatgcacatatcattttctgtgataccttccacacacccctcccccccaaaaaaatttttctgggaaagtttcttgaaaggaaaacagaagaacaagcctgtctttatgattgagttgggcttttgttttgctgtgtttcatttcttcctgtaaacaaatactcaaatgtccacttcattgtatgactaagttggtatcattaggttgggtctgggtgtgtgaatgtgggtgtggatctggatgtgggtgggtgtgtatgccccgtgtgtttagaatactagaaaagataccacatcgtaaacttttgggagagatgatttttaaaaatgggggtgggggtgaggggaacctgcgatgaggcaagcaagataaggggaagacttgagtttctgtgatctaaaaagtcgctgtgatgggatgctggctataaatgggcccttagcagcattgtttctgtgaattggaggatccctgctgaaggcaaaagaccattgaaggaagtaccgcatctggtttgttttgtaatgagaagcaggaatgcaaggtccacgctcttaataataaacaaacaggacattgtatgccatcatcacaggatgtccttccttctccagaagacagactggggctgaaggaaaagccggccaggctcagaacgagccccactaattactgcctccaacagctttccactcactgcccccagcccaacatcccctttttaactgggaagcattcctactctccattgtacgcacacgctcggaagcctggctgtgggtttgggcatgagaggcagggacaacaaaaccagtatatatgattataactttttcctgtttccctatttccaaatggtcgaaaggaggaagttaggtctacctaagctgaatgtattcagttagcaggagaaatgaaatcctatacgtttaatactagaggagaaccgccttagaatatttatttcattggcaatgactccaggactacacagcgaaattgtattgcatgtgctgccaaaatactttagctctttccttcgaagtacgtcggatcctgtaattgagacaccgagtttaggtgactagggttttcttttgaggaggagtcccccaccccgccccgctctgccgcgacaggaagctagcgatccggaggacttagaatacaatcgtagtgtgggtaaacatggagggcaagcgcctgcaaagggaagtaagaagattcccagtccttgttgaaatccatttgcaaacagaggaagctgccgcgggtcgcagtcggtggggggaagccctgaaccccacgctgcacggctgggctggccaggtgcggccacgcccccatcgcggcggctggtaggagtgaatcagaccgtcagtattggtaaagaagtctgcggcagggcagggagggggaagagtagtcagtcgctcgctcactcgctcgctcgcacagacactgctgcagtgacactcggccctccagtgtcgcggagacgcaagagcagcgcgcagcacctgtccgcccggagcgagcccggcccgcggccgtagaaaaggagggaccgccgaggtgcgcgtcagtactgctcagcccggcagggacgcgggaggatgtggactgggtggac

SEQ ID NO: 40 - protein, 10 aa, Homo sapiens
ELLELDKWAS

SEQ ID NO: 41 - protein, 10 aa, Artificial Sequence
  Synthetic construct
LEEELEKWAK

SEQ ID NO: 42 - DNA, 32 bp, Artificial Sequence
  Synthetic construct
ctggaggaagagcttgaaaaatgggctaagat

SEQ ID NO: 43 - protein, 25 aa, Artificial Sequence
  Synthetic construct
ELLELDKWASLWNWFDITNWLWYIK